(19) United States
(12) Patent Application Publication
(10) Pub. No.: US 2026/0070967 A1
(43) Pub. Date: Mar. 12, 2026

(54) AFFIBODY-BASED DUAL AFFINITY FUSION PROTEINS AND USES THEREOF

(71) Applicant: University of Oregon, Eugene, OR (US)

(72) Inventors: Malvika Singhal, Eugene, OR (US); Marian H. Hettiaratchi, Eugene, OR (US)

(73) Assignee: University of Oregon, Eugene, OR (US)

(21) Appl. No.: 19/324,928

(22) Filed: Sep. 10, 2025

Related U.S. Application Data
(60) Provisional application No. 63/692,980, filed on Sep. 10, 2024.

(51) Int. Cl. C07K 16/22; A61K 39/00; A61P 19/08; C07K 16/24

(52) U.S. Cl. CPC C07K 16/22; A61P 19/08; C07K 16/243; C07K 16/247; A61K 2039/505; C07K 2317/92; C07K 2318/00; C07K 2319/01

(57) ABSTRACT

Provided are dual-affinity fusion proteins including an affibody domain specific for a therapeutic protein, and including a localization domain specific for a structural bone component. The therapeutic protein can include bone morphogenetic protein 2 (BMP-2), vascular endothelial growth factor (VEGF), fibroblast growth factor 2 (FGF-2), platelet-derived growth factor (PDGF), granulocyte-macrophage colony-stimulating factor (GM-CSF), interleukin-4 (IL-4), or glial derived neurotrophic factor (GDNF). Also provided are compositions that include the dual-affinity fusion proteins, affibodies and the corresponding therapeutic proteins, and/or a medical material used to treat a wound, or a bone or cartilage injury or disease. Also provided are methods of using the compositions, for example to treat bone injuries, bone diseases, cartilage injuries, cartilage diseases, and wounds. In some examples, the composition includes at least two different dual-affinity fusion proteins specific for the same therapeutic protein, but have different disassociation constants (K_{D}).

### CROSS REFERENCE TO RELATED APPLICATION

This application claims the benefit of U.S. Provisional Application No. 63/692,980, filed Sep. 10, 2024, which is hereby incorporated by reference in its entirety.

### ACKNOWLEDGMENT OF GOVERNMENT SUPPORT

This invention was made with government support under W81XWH-22-1-0700 awarded by the Department of Defense and R21 EB032112 awarded by the National Institutes of Health. The government has certain rights in the invention.

### FIELD

Provided are unique dual-affinity fusion proteins including an affibody sequence and a localization domain, compositions that include the dual-affinity fusion proteins and a corresponding therapeutic protein and/or a corresponding medical material, and methods of their use.

### INCORPORATION OF SEQUENCE LISTING

The Sequence Listing is submitted as an XML file in the form of the file named “sequence listing_108691-06.xml” (117,971 bytes), which was created on Sep. 10, 2025 which is incorporated by reference herein.

### BACKGROUND

Bone morphogenetic protein-2 (BMP-2) is an integral protein for bone and cartilage repair.^{1,2} It has chemotactic properties that aid in the recruitment of osteoblasts and mesenchymal stromal cells,³⁻⁵ as well as morphogenic properties that differentiate mesenchymal stromal cells towards osteogenic phenotypes.^{1,6} Because of its ability to promote bone formation, BMP-2 has been used clinically as a bone graft substitute that is delivered from an implanted absorbable collagen sponge.^{2,7} Current critical bone defect treatments deliver supraphysiological BMP-2 doses in a collagen sponge to bypass BMP-2's short half-life. However, the absorbable collagen sponge relies primarily on weak electrostatic interactions to physically entrap BMP-2, giving it a limited ability to retain BMP-2 compared to other materials.^{8,9} Uncontrolled release of BMP-2 from collagen sponges has led to reduced efficiency of BMP-2-mediated osteogenesis¹⁰ and numerous adverse effects, including swelling, soft tissue inflammation, and ectopic bone formation.^{2,11,12}

Consequently, there is a need to develop methods to improve control over BMP-2 delivery to improve the efficacy and reduce the side effects of clinical bone regeneration therapies.

Methods to control protein release from biomaterial delivery vehicles include physical modifications such as changing the porosity or degradation rate of the delivery vehicle^{13,14} and chemical modifications such as tethering proteins directly to the delivery vehicle.¹⁵ While promising under certain conditions, these techniques often result in burst release kinetics, unpredictable protein release rates within complex in vivo environments, and inconsistent loading of the protein therapeutic, contributing to insufficient localization of the protein in the intended site and poor healing outcomes.¹⁶ Although chemical conjugation of therapeutic proteins to biomaterials can reduce the likelihood of burst release and prolong protein presentation within the site of interest,¹⁷ it can also interfere with protein-receptor binding, potentially altering the biological function of the therapeutic protein and resulting in reduced protein bioactivity.¹⁸⁻²⁰

To better address the need for controlled protein delivery, biomaterial delivery vehicles have been fabricated from extracellular matrix molecules, such as heparin^{21,22} and fibronectin²³ with intrinsic affinity interactions for therapeutic proteins in an effort to provide sustained protein delivery without directly conjugating the protein to the delivery vehicle.^{11,24,25} Affinity-mediated protein release relies primarily on the equilibrium dissociation constant (K_{D}) between the protein and material to control the rate of protein release.²⁶ These reversible interactions provide prolonged protein presentation within the site of biomaterial implantation that mimics that of the extracellular matrix. Additional affinity interactions for therapeutic proteins have been engineered using various types of affinity molecules, including antibodies,²⁷ antibody fragments,⁷ peptides,^{28,29} and aptamers.³⁰

A challenge remains in finding ideal candidates to engineer suitable protein-material interactions for affinity-based release. Heparin-containing delivery vehicles have an intrinsic affinity for BMP-2 and the ability to retain BMP-2 at the site of injury^{7,31}; however, heparin interacts with a plethora of other proteins and extracellular matrix molecules via electrostatic and hydrophobic interactions,³² making it difficult to predict the behavior of the delivery vehicle in complex in vivo environments that contain numerous serum-borne proteins. While antibodies are highly specific protein binders with high affinities for their targets, they are expensive to produce and bulky, which hinders the integration of sufficient quantities of antibodies into a delivery vehicle.^{33 34} Conversely, aptamers, peptides, and antibody fragments are smaller and less expensive to produce, but may suffer from lower specificity and/or weaker affinities for their respective proteins due to either smaller interfaces of interaction^{35,36} or non-specific targeting domains such as the heparin-binding domain.^{32,37}

Directed evolution can be used to identify highly specific protein binders.³⁸ For instance, a large yeast surface display library containing ˜10⁸ unique protein binders was subjected to directed evolution to identify highly specific protein binding partners.^{39,40} Similarly, directed evolution of a phage display library of random 7-mer peptides identified several binding peptides.⁴¹

### SUMMARY

The present disclosure provides dual-affinity fusion proteins, which include an affibody sequence, a linker, and a localization domain, wherein the affibody sequence is specific for a therapeutic protein, and the localization domain is specific for a structural bone component, which can be synthetic or natural, as well as organic or inorganic. The disclosed dual-affinity fusion proteins can be readily assembled and used in combination with known medical material such as a collagen scaffold, collagen sponge, bone void filler, bone autograft, bone allograft, or bone implant, because the localization domain will bind the dual-affinity fusion proteins to these materials. The affibody sequence of the fusion protein binds to a therapeutic protein, and the reversible binding allows for controlled release of the therapeutic protein. Therefore, when using the fusion protein to deliver the therapeutic protein to a subject, problems caused by a sudden spike of the therapeutic protein in the subject can be reduced or avoided.

Unique BMP-2-specific affibodies were identified that bound to BMP-2 with different affinities. Biolayer interferometry revealed an equilibrium dissociation constant (K_{D}) of 10.7 nM for the interaction between BMP-2 and a high-affinity affibody (SEQ ID NO: 1), 10.4 nM for the interaction between BMP-2 and a medium-affinity affibody (SEQ ID NO: 2), and 34.8 nM for the interaction between BMP-2 and a low-affinity affibody (SEQ ID NO: 3). The low-affinity affibody-BMP-2 interaction also exhibited an off-rate constant that was an order of magnitude higher than the off-rate constants of the medium- and high-affinity affibodies. Computational modeling of affibody-BMP-2 binding predicted that the high- and low-affinity affibodies bind to two distinct sites on BMP-2 that function as different cell-receptor binding sites. BMP-2 binding to affibodies reduced expression of the osteogenic marker alkaline phosphatase (ALP) in C2C12 myoblasts. Similar studies were conducted to identify and test affibodies for other proteins, including granulocyte macrophage colony-stimulating factor (GM-CSF), vascular endothelial growth factor (VEGF), fibroblast growth factor-2 (FGF-2), platelet-derived growth factor (PDGF), interleukin-4 (IL-4), and glial derived neurotrophic factor (GDNF). In some examples, the affibody sequence of the dual-affinity fusion protein includes one or more of SEQ ID NOs: 1-68 and 71-74. In some examples, the affibody sequence of the dual-affinity fusion protein is one having at least 90%, at least 95%, at least 96%, at least 97%, at least 98%, at least 99%, or 100% sequence identity to any of SEQ ID NOs: 1-68 and 71-74. In some examples, the affibody sequence of the dual-affinity fusion proteins is one consisting of any of SEQ ID NOs: 1-68 and 71-74. In some examples, the affibody sequence of the dual-affinity fusion protein includes one or more of SEQ ID NOs: 1-68 and 71-74, with 1, 2, 3, 4, 5 or 6 conservative amino acid substitutions.

The localization domain of the dual-affinity fusion protein specifically and non-covalently binds to a structural bone component. Such a structural bone component can be natural or synthetic. In one example, a structural bone component is organic. In one example, a structural bone component is inorganic. Specific examples of structural bone components that the localization domain can non-covalently and specifically bind to include but are not limited to collagen (such as a Type I collagen), a calcium phosphate (such as hydroxyapatite (HA)), or a calcium sulphate. Such structural bone components are typically included in medical material used in clinical treatments of wounds and/or bone or cartilage injuries or diseases. As such, the dual-affinity fusion protein can be, for example, incorporated into or attached to the medical material through the specific affinity interactions between the localization domain of the fusion protein and the structural bone component contained in the medical material. Exemplary medical materials include a collagen scaffold, collagen sponge, bone void filler, bone autograft, bone allograft, or bone implant. In one aspect, the localization domain specifically binds to a Type I collagen. In one aspect, the localization domain specifically binds to HA. Exemplary localization domains that can be used in the dual-affinity fusion protein include one or more of SEQ ID NOs: 75-79 and 127. In some examples, the localization domain of the dual-affinity fusion protein includes one or more of SEQ ID NOs: 75-79 and 127 with 1, 2, 3, 4, 5 or 6 conservative amino acid substitutions.

The linker of the dual-affinity fusion protein is used to join the localization domain and affibody together. In some examples, one or more linkers separate the localization domain and the affibody so that neither the localization domain nor the affibody occludes or interferes with the binding of the other. In some aspects, the linker is a flexible linker, a rigid linker, or a hybrid linker having both flexible and rigid segments. In one aspect, the linker is a rigid linker. Exemplary linkers that can be used in the dual-affinity fusion protein include one or more of SEQ ID NOs: 80-85. In some examples, the linker of the dual-affinity fusion protein includes one or more of SEQ ID NOs: 80-85 with 1, 2, 3, 4, 5 or 6 conservative amino acid substitutions.

Any of the affibody sequences (including variants thereof) disclosed herein can be linked to any of the localization domain sequences (including variants thereof) disclosed herein, through any of the linker sequences (including variants thereof) disclosed herein, to form the dual-affinity fusion proteins. The fusion proteins can optionally include an His-tag (e.g., SEQ ID NO: 86) at its terminus (e.g., N- or C-terminal). In some examples, the fusion protein exhibits a higher affinity (e.g., a K_{D} of 1-50 nM) towards the structural bone component than towards the therapeutic protein (e.g., a K_{D} of >10, >20, >50, or >100 nM). In some examples, the fusion protein exhibits a lower affinity towards the structural bone component (e.g., a K_{D} of 200-500 nM) than towards the therapeutic protein (e.g., a K_{D} of 10⁻¹⁰⁰ nM); for example, the K_{D} for therapeutic protein binding is less than an order of magnitude lower than that for structural bone component binding. Exemplary dual-affinity fusion proteins include those set forth in SEQ ID NOs: 87-112 and 126. In one aspect, a dual-affinity fusion protein has at least 90%, at least 95%, at least 96%, at least 97%, at least 98%, at least 99%, or 100% sequence identity to any of SEQ ID NOs: 87-112 and 126. In one aspect, a dual-affinity fusion protein consists of any of SEQ ID NOs: 87-112 and 126. In one aspect, a dual-affinity fusion protein includes any of SEQ ID Nos: 87-112 and 126 with 1, 2, 3, 4, 5, 6, 7, 8, 9, 10, 11, 12, 13, 14, 15, 16, 17, 18, 19, or 20 conservative amino acid substitutions. Exemplary coding sequences for these proteins include those set forth in SEQ ID NOs: 113-119.

The present disclosure provides compositions that include (1) one or more dual-affinity fusion proteins provided herein, and one or more therapeutic proteins, (2) one or more dual-affinity fusion proteins provided herein, one or more therapeutic proteins, and a medical material for treating wound, and/or bone or cartilage injuries or diseases, or (3) one or more dual-affinity fusion proteins provided herein, and a medical material for treating wound, and/or bone or cartilage injuries or diseases. The affibodies in the one or more dual-affinity fusion proteins are specific for the one or more therapeutic proteins, and the localization domain in the one or more fusion proteins is specific for a structural bone component of the medical material. In some aspects, the one or more therapeutic proteins are non-covalently bound to the one or more affibodies of the one or more fusion proteins. For example, if the affibody is specific for BMP-2, the composition can include BMP-2 bound to one or more different BMP-2-specific affibodies of the one or more fusion proteins. In some examples, the one or more dual-affinity fusion proteins are non-covalently bound to (e.g., contained within or bound to the surface of) the medical material. Such compositions can further include a pharmaceutically acceptable carrier, such as water or saline or a buffer. In some examples, such compositions can be used to control the release of the one or more therapeutic proteins.

In some examples, the composition includes one or more, two or more, or three or more of bone morphogenetic protein 2 (BMP-2) protein, vascular endothelial growth factor (VEGF) protein (such as VEGF₁₆₅), fibroblast growth factor 2 (FGF-2) protein, platelet-derived growth factor (PDGF) protein (such as PDGF-BB), granulocyte-macrophage colony-stimulating factor (GM-CSF) protein, interleukin-4 (IL-4) protein, and glial derived neurotrophic factor (GDNF) protein, and corresponding fusion proteins including affibodies specific for one or more of BMP-2, VEGF, FGF-2, PDGF, GM-CSF, IL-4, and GDNF. Exemplary affibody sequences that can be used are provided in SEQ ID NOs: 1-68 and 71-74. In some examples, the composition includes at least two fusion proteins including affibodies specific for at least two of BMP-2, VEGF (such as VEGF₁₆₅), FGF2, PDGF (such as PDGF-BB), GM-CSF, IL-4, and GDNF (and at least two of the corresponding proteins). In some examples, the composition includes the following proteins and one or more fusion proteins including affibodies specific for the protein: a) VEGF, FGF2, and PDGF-BB; b) GM-CSF; c) GDNF; d) VEGF, FGF2, PDGF-BB, and BMP-2; e) GM-CSF and IL-4; f) GM-CSF, IL-4 and MCP-1; g) GM-CSF, IL-4, and BMP-2; h) VEGF and BMP-2; or i) PDGF and BMP-2.

The compositions can include additional proteins such as monocyte chemoattractant protein-1 (MCP-1), and corresponding dual-affinity fusion proteins. In some examples, the composition further includes one or more additional chemoattractant proteins (e.g., MCP-1, SDF-1a) and corresponding dual-affinity fusion proteins, cytokine proteins (e.g., IL-10) and corresponding dual-affinity fusion proteins, immunomodulatory proteins (e.g., IL-10, MCP-1, G-CSF) and corresponding dual-affinity fusion proteins, and/or morphogen proteins (e.g., NGF, NT-3, BDNF) and corresponding dual-affinity fusion proteins.

The composition can include at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 20, 25, 30, 35, 40, 45, 50, 75, 100 or more) different dual-affinity fusion proteins that contain affibodies specific for a single therapeutic protein at different affinities. In such examples, each dual-affinity fusion protein includes a unique affibody can have a unique affinity or K_{D} for the target protein, such as at least one with a low K_{D}/strong affinity (e.g., K_{D} about 10⁻⁹-10⁻⁸ M), at least one with a medium K_{D}/medium affinity (e.g., K_{D} about 10⁻⁷-10⁻⁶ M) and at least one with a higher K_{D}/weak affinity (e.g., K_{D} about 10⁻⁵-10⁻³ M). In some examples, each dual-affinity fusion protein containing a unique affibody has a unique affinity or K_{D} for the target protein, such as at least one with a low K_{D}/strong affinity (e.g., K_{D} about 10⁻⁹-10⁻⁸ M) and at least one with a higher K_{D}/weak affinity (e.g., K_{D} about 10⁻⁵-10⁻³ M). In some examples, each dual-affinity fusion protein containing a unique affibody has a K_{D} for the target protein that is at least an order of magnitude (e.g., at least about 10 fold) different from another unique affibody. Thus, in some examples a low K_{D}/strong affinity dual-affinity fusion protein has a K_{D} that is at least about 10 times lower than a medium K_{D}/medium affinity affibody, and a medium K_{D}/medium affinity affibody has a K_{D} that is at least about 10 times lower than a higher K_{D}/weak affinity affibody.

In some examples, the composition includes at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 25, 30, 35, 40, 45, 50, 75, 100 or more) different dual-affinity fusion proteins including unique affibodies, wherein each unique affibody is specific for a single therapeutic protein. In some examples, combinations are used (e.g., two or more fusion proteins including affibodies specific for therapeutic protein 1, and two or more fusion proteins including affibodies specific for therapeutic protein 2, etc.). In some examples, where 2 or more fusion proteins including unique affibodies are present that are specific for the same protein, each fusion protein can have a distinct K_{D}, such as one with a higher and another with a lower K_{D} (such as at least 2-fold, at least 3-fold, at least 5-fold, or at least 10-fold lower).

In some examples, the localization domain of the fusion protein specifically binds to a collagen, a calcium phosphate, or a calcium sulphate, which is included in the medical material. In some examples, the collagen is Type I collagen, Type II collagen, or Type III collagen. In some examples, the calcium phosphate is hydroxyapatite (HA), β-tricalcium phosphate (β-TCP), α-tricalcium phosphate (α-TCP), calcium deficient hydroxyapatite (CDHA), tetracalcium phosphate (TTCP), amorphous calcium phosphate (ACP), dicalcium phosphate dihydrate (DCPD), or dicalcium phosphate anhydrous (DCPA). In some examples, the medical material is a collagen scaffold, collagen sponge, bone void filler, bone autograft, bone allograft, or bone implant.

Also provided are methods of using the disclosed dual-affinity fusion proteins and compositions to treat a wound, or a bone or cartilage injury or disease, by administering one or more dual-affinity fusion proteins, such as one or more compositions that include the dual-affinity fusion protein and an effective amount of the one or more corresponding therapeutic proteins, to a subject in need thereof. For example, a medical material used to provide structural support or function as a scaffold for bone growth and/or wound healing can incorporate one or more therapeutic proteins (such as BMP-2, VEGF, FGF-2, PDGF, GM-CSF, IL-4, or any combination thereof) through one or more dual-affinity fusion proteins, to facilitate bone growth and/or wound healing, through the action of the therapeutic proteins. For example, BMP-2 stimulates the differentiation of mesenchymal stem cells into osteoblasts and chondrocytes, which are essential for bone and cartilage formation; FGF-2 and PDGF attract osteoblasts and fibroblasts among other cells to the injury site and stimulates their proliferation, contributing to tissue repair and regeneration; VEGF, FGF-2, and PDGF promote angiogenesis, enhancing blood supply to the healing tissue; GM-CSF and IL-4 modulate immune responses to create a balanced environment for effective tissue repair and regeneration. Such medical material incorporating one or more therapeutic proteins through one or more dual-affinity fusion proteins can release the therapeutic proteins in a controlled manner without causing (or reducing) the problem of a burst. Different dual-affinity fusion proteins with different affinities for the same therapeutic protein can be combined at certain ratios to fine-tune the release rate of the therapeutic proteins. In some examples, a disclosed composition is administered to the site of injury, for example, during a surgical procedure, such as an orthopedic procedure.

The foregoing and other features of this disclosure will become more apparent from the following detailed description of several aspects which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E: Identification of BMP-2-specific affibodies using cell sorting of a yeast surface display library. FIGS. 1A-1B: Magnetic activated cell sorting. FIG. 1A: Schematic of MACS. Yeast induced to express surface-displayed affibodies were incubated with magnetic beads coated with tris or bovine serum albumin (BSA) for negative bead sorts or BMP-2 for positive bead sorts. Yeast that did not bind to the negative sort beads were transferred to a tube containing the BMP-2 beads. Yeast that bound to the BMP-2 beads were collected and expanded for the next round of cell sorting. FIG. 1B: Diversities of the sorted yeast libraries obtained after each round of MACS. Left y-axis (bar graph) depicts approximate ratio of BMP-2-specific to non-specific binders (i.e., positive-to-negative binder ratio). Right y-axis (line graph) depicts the diversity of the yeast library after each round of MACS. FIGS. 1C-1E: FACS plots and corresponding cell labeling diagrams. Yeast was incubated with secondary fluorescent tags that bound specifically to αCMYC for affibody expression or bBMP. FIG. 1C: Secondary fluorescent tag control, in which no affibody expression or bBMP-2 binding is measured. FIG. 1D: In the presence of αCMYC, affibody expression is observed as a rightward shift. FIG. 1E: In the presence of αCMYC and bBMP-2, affibody expression is observed as a rightward shift, and binding of bBMP-2 to displayed affibodies results in a shift upward along the y-axis. Gating was performed based on positive bBMP-2 binding and affibody expression (upper right quadrant).

FIGS. 2A-2B: Gating approaches for fluorescent-activated cell sorting of enriched affibody-displaying yeast library. FIG. 2A: Cell sorting based on ratio of BMP-2 binding to affibody expression. Gates were created for ratios of >1, ˜1, <1, based on our hypothesis that a higher ratio of BMP-2 binding to affibody expression would correspond to higher affinity for BMP-2. 8 unique affibody sequences were identified from 9 colonies picked from yeast growth plates after sorting. FIG. 2B: Cell sorting based on BMP-2 binding only. Gates were created based on the hypothesis that higher BMP-2 binding to the yeast would correspond to higher BMP-2 affinity. 3 unique affibody sequences were identified from the 12 colonies picked from yeast growth plates after sorting.

FIGS. 3A-3H: Identification and characterization of yeast-displayed BMP-2-specific affibodies. Affibody binding to BMP-2 and off-target proteins was determined using flow cytometry. Flow cytometry plots of affibodies binding to bBMP-2 at 0.5 nM of bBMP-2 (FIG. 3A), 5 nM of bBMP-2 (FIG. 3B), 50 nM of bBMP-2 (FIG. 3C), and 500 nM of bBMP-2 (FIG. 3D). FIG. 3E: Fraction of yeast binding to bBMP-2 as a function of bBMP-2 concentration ranging from 0.5-1000 nM. Non-linear regression was performed to determine equilibrium dissociation constants (K_{D}). Curves were identified as statistically significantly different from each other by two-way ANOVA, Tukey's post-hoc test. n=3, p<0.01. FIG. 3F: Equilibrium dissociation constants (K_{D}) of the three unique affibodies. Statistical significance was determined using one-way ANOVA with Tukey's post-hoc test. n=3, ** p<0.01 FIGS. 3G-3H: BMP-2-specific affibodies do not bind to other recombinant proteins of interest. High-affinity (FIG. 3G) and low-affinity affibodies (FIG. 3H) were incubated with 1000 nM of bBMP-2, bVEGF, bIL-4, or bGM-CSF. Statistical significance was measured using one-way ANOVA and Tukey's post-hoc test. n=3, **** p<0.0001

FIGS. 4A-4B: bBMP-2 binding to selected affibody-displaying yeast clones from enriched yeast library. FIG. 4A: All 11 unique BMP-2 affibodies (SEQ ID NOS: 1-11) were assessed for their binding to bBMP-2 using yeast surface display, bBMP-2 concentrations ranging from 0.5-1000 nM, and flow cytometry. Yeast clones with names starting with the letter A were identified using the gating approach in FIG. 2A, and yeast clones with names starting with B were identified using the gate approach in FIG. 2B. The affibodies chosen for further characterization are boxed in the legend. FIG. 4B: Average AlexaFluor647 signal intensity for yeast displaying high-affinity and low-affinity BMP-2 affibodies at BMP-2 concentrations between 0.5 nM and 500 nM, as determined by flow cytometry. No significant differences were observed using a non-parametric multiple t test with Mann-Whitney post-hoc analysis. n=4, p=0.6857.

FIGS. 5A-5B: Soluble affibody characterization. FIG. 5A: SDS-PAGE of high- and low-affinity BMP-2-specific affibodies with a 5-245 kDa ladder. Samples were run at concentration of 150 μM. Expected molecular weights of the high- and low-affinity affibodies were 7308 Da and 7414 Da, respectively. FIG. 5B: Circular dichroism spectra of affibodies displayed in molar ellipticity. Affibodies were diluted to a concentration between 17-30 μM in 5 mM tris pH 6.92 and loaded into a quartz cuvette with 1 mm path length. Circular dichroism and high-tension voltage were measured over a wavelength range of 190-250 nm, and the circular dichroism output was adjusted for protein concentration and molecular weight.

FIG. 6: Deconvolved mass spectra relative abundance of high-affinity and low-affinity affibodies. Accurate masses were measured using deconvolved native mass spectrometry collected with Waters Synapt G2Si, using a CsI calibration profile. Purified affibodies were dissolved in 0.6 M tris pH 8 and buffer-exchanged into 0.2 M ammonium acetate pH 7.52. Mass spectra were collected over 1-5 minutes using nano-electrospray ionization at a capillary voltage of 0.7-1.0 kV. Samples were deconvolved in UniDec using charge states of 3 to 7 and masses of 5000-9000 Da. The high-affinity affibody's (SEQ ID NO: 1) most abundant peak was 7308 Da (starred), aligned with the expected mass within 2 Da. Mass spectrometry associated adducts of sodium (22 Da) and potassium (38 Da) are also visible. Other apparent peaks could be associated with dehydroalanine (−34 Da) and either a piperidine (51 Da) or cysteic acid formation (48 Da) on the C-terminal cysteine or glutamylation (129 Da (+/−2)) of the terminal cysteine. The low-affinity affibody (SEQ ID NO: 3) had a small peak at the expected mass of 7414 Da (starred), but had prominent peak shifts associated with the dehydroalanine, a 32 Da shift which could be indicative of a proline oxidation or 3,4-dihydroxylation, an additional shift associated with a piperidine formation, and another prominent peak (161 Da) which could be caused by a carboxymethyl cysteine or carboxymethyl cystenyl.

FIGS. 7A-7C: Binding interactions of soluble affibodies with BMP-2 measured by biolayer interferometry. All samples were diluted in PBST. Streptavidin probes were loaded with 25 nM of bBMP-2 for 120 seconds. Bound protein was allowed to associate with 0-125 nM high-affinity affibody (SEQ ID NO: 1) (FIG. 7A) and low-affinity affibody (SEQ ID NO: 3) (FIG. 7B) for 120 seconds followed by dissociation into PBST for 120 seconds. Association and dissociation rate constants as well as overall equilibrium dissociation constants were obtained using a 1:1 global curve fitting of the data. Raw data is displayed with solid lines, and fitted data is displayed as dotted lines. FIG. 7C: Table of association and dissociation rate constants and overall equilibrium dissociation constants of high-, medium- and low-affinity BMP-2 affibodies (SEQ ID NOS: 1, 2, and 3, respectively).

FIGS. 8A-8F: Binding interactions of soluble BMP-2-specific affibodies with VEGF, IL-4, and GM-CSF measured by biolayer interferometry. All samples were diluted in PBST. Streptavidin probes were loaded with 25 nM of bVEGF (FIGS. 8A & 8D), bIL-4 (FIGS. 8B & 8E), or bGM-CSF (FIGS. 8C & 8F) for 120 seconds. Bound protein was allowed to associate with 0-125 nM high-affinity (SEQ ID NO: 1) (FIGS. 8A-8C) or low-affinity (SEQ ID NO: 3) (FIGS. 8D-8F) affibodies for 120 seconds followed by dissociation into PBST for 120 seconds.

FIGS. 8G-8H: Binding interactions of soluble medium-affinity BMP-2 affibody (SEQ ID NO: 2) with BMP-2 and VEGF measured by biolayer interferometry. All samples were diluted in PBS solution containing 0.05% Tween-20® (PBST). Streptavidin probes were loaded with 25 nM biotinylated BMP-2 for on-target binding (FIG. 8G) and 25 nM biotinylated VEGF for off-target binding (FIG. 8H) for 120 seconds, excess protein was allowed to dissociate in PBST for 120 seconds, then bound protein was allowed to associate with 0-125 nM medium-affinity BMP-2 affibodies for 120 seconds and dissociate into PBST for 120 seconds. Dissociation constants were obtained from a 1:1 global curve fitting of the data. n=4.

FIGS. 9A-9B: Computational predictions of BMP-2 binding with affibodies or BMP receptors. FIG. 9A: Visual representation of docking of affibodies and receptors to BMP-2 in Pymol. High-affinity affibody (SEQ ID NO: 1) (a) overlaps with the docking interface of BMPR-1A (b), and low-affinity affibody (SEQ ID NO: 3) (c) overlaps with the docking interface of BMPRII (d). Predicted affibody structures were docked to BMP-2 using ZDOCK. FIG. 9B: Characteristic binding interactions of affibodies and BMP receptors to BMP-2. The high-affinity affibody (SEQ ID NO: 1) docks to BMP-2 in the binding epitope known at the “wrist” using 3 polar contacts and 5 hydrophobic interactions. The low-affinity affibody (SEQ ID NO: 3) docks to BMP-2 at the binding epitope called the “knuckle” with 3 polar interactions and one weak hydrophobic pocket. The wrist traditionally binds BMPR-1A (PDB: 1REW) and the knuckle traditionally binds with BMPR-II (PDB: 7PPA).

FIGS. 10A-10F: Effects of BMP-2 affibodies on viability, growth, and alkaline phosphate activity of C2C12 skeletal myoblasts. FIGS. 10A-10E: Cytocompatibility of high- and low-affinity affibodies with C2C12s. Cells were seeded at a density of 2000 cells cm⁻² and incubated with 0-800 nM soluble affibody in growth media for 72 hours. Cells were stained with calcein AM (live cells; light gray) and ethidium homodimer (dead cells; starred) and imaged. FIGS. 10A-10C: Representative photos of stained cell culture wells containing C2C12 cells and no affibodies (FIG. 10A), 20 nM high-affinity affibodies (FIG. 10B), and 20 nM low-affinity affibodies (FIG. 10C). Scale bar=1000 μm. FIG. 10D: Percent cell viability as a function of affibody concentration. Significance determined by two-way ANOVA and Dunnett's post-hoc test, n=4, * p<0.05. FIG. 10E: Total viable cell number as a function of affibody concentration. Statistical significance determined by two-way ANOVA and Dunnett's post-hoc test, n=4, * p<0.05. FIG. 10F: Effects of BMP-2 and BMP-2-specific affibodies on alkaline phosphatase activity of C2C12 cells. C2C12 cells were seeded at a density of 62,500 cells cm⁻² and allowed to adhere for 6 hours in growth media. Media was then replaced with low serum media containing premixed affibody (20 nM) and BMP-2 (20 nM) (complexed) or low serum media containing 20 nM soluble affibody with 20 nM of BMP-2 added 45 minutes later (uncomplexed). The cells were cultured for 72 hours and then lysed for quantification of ALP activity and double stranded DNA content. Statistical significance was determined by one-way ANOVA and Tukey's post-hoc test. n=4, * p<0.05, ** p<0.01, *** p<0.001, **** p<0.0001. Complexed and uncomplexed affibody-BMP-2 structures were obtained from computational predictions.

FIG. 11: Effects of BMP-2 and BMP-2-specific affibodies on normalized alkaline phosphatase activity of C2C12 cells. C2C12 cells were seeded at an initial density of 62,500 cells/cm² and allowed to adhere for 6 hours in high serum media. Media were then replaced with low serum media containing premixed affibody (10−1000 nM) and BMP-2 (20 nM) (complexed) or low serum media containing 10−1000 nM soluble affibody with 20 nM of BMP-2 added 45 min later (uncomplexed). The cells were cultured for 72 hours and then lysed. ALP activity and double stranded DNA content of lysates were quantified, and ALP activity was normalized to dsDNA content. Statistical significance was determined by one-way ANOVA and Tukey's post-hoc test. n=4, * p<0.05, ** p<0.01, *** p<0.001, **** p<0.0001

FIGS. 12A-12D show characterization of GM-CSF affibodies. FIG. 12A: yeast displaying GM-CSF affibodies incubated with increasing amounts of biotinylated GM-CSF. FIG. 12B: Yeast displaying GM-CSF affibodies incubated with increasing amounts of biotinylated high-affinity GM-CSF affibody (SEQ ID NO: 12). FIG. 12C: Yeast displaying GM-CSF affibodies incubated with increasing amounts of biotinylated medium-affinity GM-CSF affibody (SEQ ID NO: 13). FIG. 12D: Yeast displaying GM-CSF affibodies incubated with increasing amounts of biotinylated low-affinity GM-CSF affibody (SEQ ID NO: 14).

FIGS. 13A-13C are graphs showing yeast displaying high-, medium-, and low-affinity affibodies incubated with increasing amounts of biotinylated FIG. 13A: VEGF, FIG. 13B: FGF-2, and FIG. 13C: PDGF.

FIGS. 13D-13F are graphs showing yeast displaying high-, medium-, and low-affinity affibodies incubated with increasing amounts of biotinylated FIG. 13D: VEGF, FIG. 13E: FGF-2, and FIG. 13F PDGF, showing specificity of each affibody for its target protein and any off-target binding to other proteins.

FIG. 13G is a graph showing biolayer interferometry data characterizing the dissociation constant between PDGF and a high-affinity PDGF affibody (SEQ ID NO: 59).

FIG. 14 is a graph showing results of circular dichroism: the BMP-2 specific affibody sequence in the dual-affinity fusion protein (SEQ ID NO: 87) retains an alpha-helical structure with two characteristic troughs observed at 208 nm and 221 nm.

FIG. 15 is a graph showing results of biolayer interferometry (BLI) using a Ni-NTA probe for 125 nM collagen: Type I collagen does not non-specifically bind to the Ni-NTA probe.

FIG. 16A is a graph showing results of BLI using a Ni-NTA probe loaded with 25 nM His-tagged (SEQ ID NO: 86) collagen binding domain (SEQ ID NO: 75) for Type I collagen at serially diluted concentrations from 125 nm to 3.9 nm: the collagen binding domain binds to Type I collagen in a concentration dependent manner.

FIG. 16B is a graph showing results of BLI using a Ni-NTA probe loaded with 62.5 nM His-tagged (SEQ ID NO: 86) collagen binding domain (SEQ ID NO: 75) for 25 nM BMP-2: the collagen binding domain does not bind non-specifically to BMP-2.

FIG. 17A is a graph showing results of BLI using a Ni-NTA probe loaded with a His-tagged (SEQ ID NO: 86) fusion protein (SEQ ID NO: 87) at serially diluted concentrations from 500 nM to 15.625 nM, for 25 nM biotinylated BMP-2: the fusion protein binds to BMP-2 in a concentration dependent manner.

FIG. 17B is a graph showing results of BLI using a Ni-NTA probe loaded with 125 nM His-tagged (SEQ ID NO: 86) fusion protein (SEQ ID NO: 87) for 25 nM biotinylated FGF-2 (bFGF-2), loaded with 125 nM of the His-tagged fusion protein for 0 nM bFGF-2, or loaded with 0 nm of the His-tagged fusion protein for 25 nm bFGF-2: the fusion protein does not bind non-specifically to FGF-2.

FIG. 17C is a graph showing results of BLI using a Ni-NTA probe loaded with 125 nM His-tagged (SEQ ID NO: 86) fusion protein (SEQ ID NO: 87) for 25 nM biotinylated VEGF (bVEGF), loaded with 125 nM of the His-tagged fusion protein for 0 nM bVEGF, or loaded with 0 nm of the His-tagged fusion protein for 25 nm bVEGF: the fusion protein does not bind non-specifically to VEGF.

FIG. 18 is a graph showing results of BLI using a Ni-NTA probe loaded with 62.5 nM His-tagged (SEQ ID NO: 86) collagen binding domain (SEQ ID NO: 75) for 25 nM BMP-2, loaded with 62.5 nM His-tagged (SEQ ID NO: 86) collagen binding domain (SEQ NO: 76) for 25 nM BMP-2, or loaded with 0 nm His-tagged (SEQ ID NO: 86) collagen binding domain (SEQ ID NO: 76) for 25 nM BMP-2: both collagen binding domains do not bind non-specifically to BMP-2.

FIG. 19 shows structural models of BMP-2 binding by five fusion proteins with collagen-binding domains, each having a different linker sequence. BMP-2 binding to the five fusion proteins was predicted using computational protein modeling.

FIG. 20 illustrates a fluorescence retention assay for testing the binding capability of the collagen-binding and BMP-2 binding fusion proteins to collagen.

FIGS. 21A-21C are gel electrophoresis images of fusion proteins demonstrating size and purity. 21A: lane 1: kDa ladder; lane 2: blank; lane 3: flowthrough; lane 4: wash; lane 5: elution; lane 6: dialysate at approximate 8500 kDa. 21B: lane 1: kDa Ladder; lane 2: blank; lane 3: post-sonication supernatant; lane 4: flowthrough; lane 5: wash; lane 6: elution; lane 7: concentrated protein in 1×PBS at approximate 8300 kDa. 21C: lane 1: kDa ladder; lane 2: flowthrough; lane 3: wash; lane 4: elution; lane 5: dialysate at approximate 9200 kDa; lane 6: blank; lane 7: flowthrough; lane 8: wash; lane 9: elution; lane 10: dialysate at approximate 8900 kDa.

FIG. 22 is a circular dichroism spectra of fusion proteins.

FIGS. 23A-23B illustrate predicted structures and interactions of fusion proteins with HA-binding domains. 23A: AlphaFold3 structure renderings of fusion proteins visualized in PyMOL. 23B: Serine interactions in SVSVSVK binding domain (tan) with positively charged calcium ions in HA (green) visualized in PyMOL.

FIG. 24 illustrates a fluorescence retention assay for testing the binding capability of the hydroxyapatite (HA)-binding and BMP-2 binding fusion proteins to HA.

FIGS. 25A-25E show affinities of a BMP-2 affibody (SEQ ID NO: 1) for BMP-2, a collagen binding domain (SEQ ID NO: 75) for collagen, and a fusion protein (SEQ ID NO: 87) for BMP-2 and collagen.

FIGS. 26A-26F show dual binding interactions of a fusion protein (SEQ ID NO: 87) to BMP-2 and collagen at different concentrations.

FIG. 27 show retention to collagen sponge of an affibody (SEQ ID NO: 1), a collagen-binding domain (SEQ ID NO: 75), a collagen-binding fusion protein (SEQ ID NO: 87), and an HA-binding fusion protein (SEQ ID NO: 108).

FIG. 28A show constructions of six HA-binding fusion proteins (SEQ ID NOs: 108-112 and 126).

FIG. 28B show CD spectra of the six HA-binding fusion proteins.

FIGS. 29A-29D show retention to HA pellets of BMP-2 affibody, or the six HA-binding fusion proteins, compared to the HA-binding domain alone.

FIGS. 30A-30B show BMP-2 release from the six HA-binding fusion proteins bound to HA pellets.

FIGS. 31A-31B show BMP-2 release from the six HA-binding fusion proteins bound to allografts.

### SEQUENCE LISTING

The amino acid sequences provided herein are shown using standard three letter code for amino acids, as defined in 37 C.F.R. 1.822.

SEQ ID NOs: 1-11 are exemplary BMP-2 affibody sequences.

SEQ ID NOs: 12-19 are exemplary GM-CSF affibody sequences.

SEQ ID NOs: 20-41 and 71-73 are exemplary VEGF-165 affibody sequences.

SEQ ID NOs: 42-56 are exemplary FGF affibody sequences.

SEQ ID NOs: 57-60 and 74 are exemplary PDGF-BB affibody sequences.

SEQ ID NOs: 61 to 64 are exemplary IL-4 affibody sequences.

SEQ ID NOs: 65-67 are exemplary BMP-2 affibody sequences with a hexahistidine tag and C-terminal cysteine.

SEQ ID NO: 68 is an exemplary GM-CSF affibody sequence with a hexahistidine tag and C-terminal cysteine.

SEQ ID NOS: 69 and 70 are primer sequences.

SEQ ID NOs: 75-79 and 127 are exemplary localization domain sequences.

SEQ ID NOs: 80-85 and 120-125 are exemplary linker sequences.

SEQ ID NO: 86 is an exemplary optional His tag sequence.

SEQ ID NOs: 87-112 and 126 are exemplary dual-affinity fusion protein sequences.

SEQ ID NOs: 113-119 are exemplary dual-affinity fusion protein coding sequences.

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used.

The sequences associated with all GenBank® Accession numbers referenced herein are incorporated by reference for the sequence available on Jul. 8, 2022.

In order to facilitate review of the various embodiments of the disclosure, the following explanations of specific terms are provided:

Administration: Administration of a composition, such as a medical material composition provided herein, can be by any route known to one of skill in the art. Administration can be local. Examples of local administration include, but are not limited to, topical administration, subcutaneous administration, intramuscular administration, intrathecal administration, intrapericardial administration, administration to a bone (e.g., intraosseous), administration to a tumor, or administration to a wound. Local administration also includes the incorporation of active compounds and agents into implantable devices, constructs, or medical materials, such as a collagen scaffold, collagen sponge, bone allograft, bone autograft, bone void filler, implant, which release the active agents and compounds over extended time intervals for sustained treatment effects. In some examples, administration includes placing or implanting the medical material (such as a collagen scaffold, collagen sponge, bone allograft, bone autograft, bone void filler, implant) including the fusion proteins and therapeutic proteins at the site of injury, defect, or void during a surgical procedure, such as an orthopedic procedure. In some examples, administration includes mixing the fusion protein into a cell pellet, cell aggregate, organoid, or suspension of cells in a hydrogel in vitro.

Affibody: A small protein that binds to a target proteins or peptides with varying affinity, and are therefore a member of the family of antibody mimetics. In some examples, affibody molecules include alpha helices and lack disulfide bridges. For example, an affibody can include three alpha helices with 58 amino acids, having a molar mass of about 6 kDa. In some examples, different affibodies specific for one protein each have a different K_{D} such as strong/high (10⁻⁹-10⁻⁸ M), medium (10⁻-10⁻⁶ M) and weak (10⁻⁵-10⁻³ M) affinity.

Binding affinity: Strength of non-covalent interactions between a ligand (such as an affibody, or a short peptide) and its target (such as an organic molecule, such as protein and collagen; or an inorganic compound or molecule, such as a calcium phosphate). Affinity can be quantified by calculating a dissociation constant, K_{D}.

An affibody (such as the affibody domain of a fusion protein) that “specifically binds” a protein (such as BMP-2, VEGF, FGF-2, PDGF, GM-CSF, IL-4, or GDNF) is an affibody or affibody domain that binds the protein with significantly higher (such as at least about 100 (e.g., at least about 110, 120, 130, 140, 150, 160, 170, 180, 190, 200, or 300) fold higher) affinity compared to other proteins. In some examples, an affibody or affibody domain specifically binds to a target protein with weak affinity, such as with a K_{D} that is no more than 10⁻⁵ M, such as no more than 10⁻⁴ M, no more than 10⁻³ M, or no more than 10⁻² M, such as about 10⁻⁵-10⁻³ M. In some examples, an affibody or affibody domain specifically binds to a target with moderate or medium affinity, such as with a K_{D} that is no more than 10⁻⁷ M, such as no more than 10⁻⁶ M, such as about 10⁻⁷-10⁻⁶ M. In some examples, an affibody or affibody domain specifically binds to a target with high or strong affinity, such as with a K_{D} that is at least 10⁻¹⁰ M, such as at least 10⁻⁹ M, or at least 10⁻⁸ M, such as about 10⁻¹⁰-10⁻⁸ M.

A localization domain that “specifically binds” a bone structural component is a peptide sequence that binds the component with significantly higher (such as at least about 100 (e.g., at least about 110, 120, 130, 140, 150, 160, 170, 180, 190, 200, or 300) fold higher) affinity compared to other molecules.

Bone: A rigid organ that constitutes part of the skeleton in most vertebrate animals. Bones protect the various other organs of the body, produce red and white blood cells, store minerals, provide structure and support for the body, and enable mobility. The disclosed compositions can be used to treat a bone injury, such as a fracture, for example in the spinal column, vertebrae (such as the lumbar vertebra), femur, tibia, fibula, thoracic cage, rib, clavicle, humerus, radius, ulna, tarsal bone, ilium, cranium or carpal bone.

Bone graft: A structure used to replace missing bone, fill a bone defect, and/or promote bone healing or regeneration, including allograft, autograft, xenograft, and synthetic bone graft (bone implant). Bone graft includes cortical bone grafts, cancellous bone grafts/chips, corticocancellous bone grafts, demineralized bone matrix (DBM), DBM putty, DBM paste, DBM gel, freeze-dried bone allografts (FDBA), fresh-frozen bone allografts, osteochondral allografts, bone chips, bone blocks, bone powder, etc. Allograft is bone tissue e.g., from another person other than the subject in treatment. Xenograft is bone tissue from a mammal (such as bovine or porcine). Autograft is bone tissue from the subject in treatment. Synthetic graft (bone implant) is an artificially produced biocompatible structure that mimics bone properties, such as HA powder or pellets or collagen sponges, by including components found in natural bone, such as HA and/or collagen.

Bone injury or disease: A bone injury or disease refers to any condition that affects the structure, function, or integrity of bones. This can include fractures (breaks) in bones due to trauma or stress, as well as diseases such as osteoporosis (where bones become brittle and fragile), osteomyelitis (bone infection), osteoarthritis (degeneration of joint cartilage and the underlying bone), and bone cancers like osteosarcoma.

Bone morphogenetic protein 2 (BMP-2): (e.g., OMIM 112261) A bone morphogenetic protein that plays a role in the development of bone and cartilage. It is involved in the hedgehog pathway, TGF beta signaling pathway, and in cytokine-cytokine receptor interaction. It is also involved in cardiac cell differentiation and epithelial to mesenchymal transition. Thus, PDGF affibodies can be used to control the release of BMP-2 and treat a bone injury. Exemplary BMP-2 sequences can be found in the GenBank® database (e.g., Accession Nos. NP_001191.1, AGG86667.1, NM_001200.4, NP_031579.2, and CAA81088.1). In some examples, a BMP-2 protein or coding sequence has at least 80%, at least 90%, at least 95%, at least 96%, at least 97%, at least 98%, at least 99% or 100% sequence identity to the sequence provided in NP_001191.1, AGG86667.1, NM_001200.4, NP_031579.2, or CAA81088.1.

Bone repair or regeneration: Includes osteogenesis, bone regeneration, bone repair, bone reformation, and bone remodeling.

Bone void filler: Biocompatible material used to fill bony voids or gaps of the skeletal system (e.g., extremities, posterolateral spine, and pelvis). Bone void filler typically includes calcium phosphate (e.g., HA, β-TCP, etc.), calcium sulfate, collagen (e.g., Type I collagen, Type II collagen, etc.), or any combination thereof, among other components. In some aspects, the components for forming the bone void filler are mixed in a liquid prior to use to form a cement-like substance, which hardens after being applied to the void. In some aspects, bone void filler is resorbed and is replaced by bone during the healing process.

Cartilage injury or disease: A cartilage injury or disease refers to any condition that affects the structure, function, or integrity of cartilage. This can include osteoarthritis, cartilage tears (meniscal tears, labral tears, etc.), chondromalacia patellae, osteochondritis dissecans, and costochondritis.

Collagen scaffold: A three-dimensional structure formed by collagen (e.g., Type I, Type II, Type III, Type IV, Type V, and/or Type XI collagen), or a blend of collagen and other biocompatible materials. Collagen scaffold is typically used in tissue engineering for replacing or repairing damaged tissues (e.g., bone, cartilage, etc.), and/or supporting tissue (e.g., bone, cartilage, etc.) regeneration. Collagen scaffolds can be engineered to have specific pore sizes, shapes, and mechanical properties, tailored to meet the requirements of different tissue types. Physical treatment (e.g., UV irradiation, gamma radiation, and dehydrothermal treatment (DHT)) or chemical agents (e.g., glutaraldehyde (GA), and 1-ethyl-3-(3-dimethylaminopropyl)-carbodiimide hydrochloride (EDC)) can be used to achieve intermolecular cross-linking of collagen, thus modifying the properties of the collagen scaffold. Biocompatible natural polymers (such as chitosan, silk fibroin, hyaluronic acid, alginate, etc.), synthetical polymers (such as poly (ε-caprolactone) (PCL), polylactic acid (PLA), poly (ethylene glycol) (PEG), polyglycolide (PGA), poly (lactide-co-glycolide) (PLGA), polyvinyl alcohol (PVA), etc.), and inorganic materials (such as hydroxyapatite (HA), silicate, β-tricalcium phosphate (β-TCP), etc.) can be blended with collagen to improve the performance of collagen scaffolds.

Collagen sponge: A type of collagen scaffold that is highly porous, typically used as wound dressing, or for tissue regeneration or repair. Collagen sponge can be obtained by lyophilizing a solution, dispersion, or gel of collagen (e.g., gel of physically- or covalently-crosslinked collagen). Optional crosslinking can be performed before or after lyophilizing.

Contacting: Placement in direct physical association; includes both in solid and liquid form.

Conservative variant: A protein, such as an affibody, a localization domain, or a linker sequence, containing conservative amino acid substitutions that do not substantially affect or decrease its function or properties. Conservative amino acid substitutions are those substitutions that, when made, least interfere with the properties of the original protein, that is, the structure and especially the function of the protein is conserved and not significantly changed by such substitutions. For example, an affibody provided herein that specifically binds to its corresponding protein can include at most about 1, at most about 2, at most about 5, at most about 10, at most about 15, or at most about 20 conservative substitutions and specifically bind the protein with a similar K_{D}(e.g., a change of no more than 10%, no more than 5%, or no more than 1%) than the original sequence. A localization domain provided herein that specifically binds to its corresponding structural bone component can include at most about 1, at most about 2, at most about 3, at most about 4, at most about 5, at most about 6, or at most about 7 conservative substitutions and specifically bind the structural bone component with a similar K_{D} (e.g., a change of no more than 10%, no more than 5%, or no more than 1%) than the original sequence. A linker provided herein can include at most about 1, at most about 2, at most about 3, at most about 4, or at most about 5 conservative substitutions and maintains a similar function in separating the two protein domains that it links. The term “conservative variant” also includes the use of a substituted amino acid in place of an unsubstituted parent amino acid, provided that the affibody specifically binds to its corresponding protein.

Conservative amino acid substitution tables providing functionally similar amino acids are well known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Consist of: A polypeptide of a specified amino acid sequence (such as an affibody or localization domain sequence) that does not include any additional amino acid residues. The residues in the polypeptide can be modified to include non-peptide components. The N- and/or C-terminus of a polypeptide that consists of a specified amino acid sequence can be joined (for example, by a covalent bond) to a chemical linker for conjugation chemistry. A polypeptide that consists of a specified amino acid sequence can be glycosylated and/or can include non-naturally occurring amino acids.

Dissociation constant (K_{D}): The concentration of ligand/affibody, wherein half of the ligand/affibody binding sites on the protein are occupied in the system equilibrium. It is calculated by dividing the k_{off} value by the kₒₙ value. The smaller the K_{D} value, the greater the binding affinity of the ligand/affibody for its target protein. The larger the K_{D} value, the more weakly the target protein and ligand/affibody are attracted to and bind to one another.

Numerous methods are available to calculate the K_{D} value for an affibody, and the disclosure is not limited to a particular method. In one embodiment, K_{D} is calculated by a modification of the Scatchard method described by Frankel et al., Mol. Immunol., 16:101-106, 1979. Other exemplary methods include competition radioimmunoassay, ELISA, flow cytometry, and surface plasmon resonance assays (e.g., using a BIACORES-2000 or a BIACORES-3000 (BIAcore, Inc., Piscataway, N.J.)). In some embodiments, K_{D} is measured using the Octet system (ForteBio) or Gator Bio system, which are both based on bio-layer interferometry (BLI) technology.

In some examples, an affibody has a K_{D} of 1 nM or less. In some examples, an affibody binds to a target protein, such as BMP-2, with a K_{D} of at least about 10⁻³ M, at least about 10⁻⁴ M, at least about 10⁻⁵ M, at least about 10⁻⁶ M, at least about 10⁻⁷ M, at least about 10⁻⁸ M, at least about 10⁻⁹ M, or at least about 10⁻¹⁰ M.

In some examples, a localization domain has a K_{D} of 100 nM or less for its corresponding bone structural component. In some examples, a localization domain binds to a bone structural component, such as collagen, a calcium phosphate, or a calcium sulphate, with a K_{D} of at most about 10⁻⁵ M (e.g., at most about 10⁻⁶ M, 10⁻⁷ M, 10⁻⁸ M, or 10⁻⁹ M, such as about 10⁻⁶ M to about 10⁻⁵ M, about 10⁻⁷ M to about 10⁻⁶ M, about 10⁻⁸ M to about 10⁻⁷ M, about 10⁻⁸ M to about 10⁻⁶ M, or about 10⁻⁹ M to about 10⁻⁷ M).

Dual-affinity fusion protein: A fusion protein that can bind to two different molecules through non-covalent affinity interactions through two distinct domains. In some aspects, the dual affinity fusion protein includes an affibody domain that binds specifically to a therapeutic protein, and a localization domain that binds specifically to a structural bone component, wherein the two domains are linked by a linker sequence.

Effective amount: An amount of agent, such as a therapeutic protein contained in a composition provided herein, that is sufficient to elicit a desired response, such as treating a bone injury, a bone disease, a cartilage injury, a cartilage disease, or wound in a subject. In one example, a desired response is to manipulate the immune response, increase wound healing, increase bone injury healing, increase angiogenesis, increase recruitment and differentiation of immune cells, and/or increase recruitment and differentiation of osteogenic cells. The wound, disease, or injury does not need to be completely eliminated or reduced or prevented for the method to be effective. In one example, administration of a composition including a therapeutically effective amount of one or more therapeutic proteins increases the rate of wound healing and/or the amount of wound healing, for example by at least 10%, at least 20%, at least 50%, at least 60%, at least 70%, at least 80%, at least 85%, at least 90%, at least 95%, at least 98%, at least 99% or even at least 100% (complete healing of the wound), for example as compared to a suitable control, such as the absence of the composition. In one example, administration of a composition including a therapeutically effective amount of one or more therapeutic proteins increases the rate of healing of a bone injury and/or the amount of bone injury, for example by at least 10%, at least 20%, at least 50%, at least 60%, at least 70%, at least 80%, at least 85%, at least 90%, at least 95%, at least 98%, at least 99% or even at least 100% (complete healing of the bone injury), for example as compared to a suitable control, such as the absence of the composition. In one example, administration of a composition including a therapeutically effective amount of one or more therapeutic proteins increases the rate and/or amount of differentiation of osteogenic cells, for example by at least 10%, at least 20%, at least 50%, at least 60%, at least 70%, at least 80%, at least 85%, at least 90%, at least 95%, at least 98%, at least 99%, at least 100%, at least 200%, at least 300%, or at least 500% (for example as compared to a suitable control, such as the absence of the composition). In one example, administration of a composition including a therapeutically effective amount of one or more therapeutic proteins increases angiogenesis, for example by at least 10%, at least 20%, at least 50%, at least 60%, at least 70%, at least 80%, at least 85%, at least 90%, at least 95%, at least 98%, at least 99%, at least 100%, at least 200%, at least 300%, or at least 500% (for example as compared to a suitable control, such as the absence of the composition). In one example, administration of a composition including a therapeutically effective amount of one or more therapeutic proteins increases recruitment and/or differentiation of immune cells, for example by at least 10%, at least 20%, at least 50%, at least 60%, at least 70%, at least 80%, at least 85%, at least 90%, at least 95%, at least 98%, at least 99%, at least 100%, at least 200%, at least 300%, or at least 500% (for example as compared to a suitable control, such as the absence of the composition).

Fibroblast growth factor 2 (FGF-2): (e.g., OMIM 134920) Also known as basic fibroblast growth factor (bFGF) and FGF-β. A growth factor and signaling protein that binds to and exerts effects via specific fibroblast growth factor receptor (FGFR) proteins, a family of closely related molecules. FGF-2 is involved in cellular proliferation, wound healing and angiogenesis. Thus, FGF-2 affibodies can be used to control the release of FGF-2 and increase angiogenesis, for example to treat a wound or vascular disease. Exemplary FGF-2 sequences can be found in the GenBank® database (e.g., Accession Nos. NP_001997.5, NM_002006.6, NP_001348594.1, and NP_032032.1). In some examples, an FGF-2 protein or coding sequence has at least 80%, at least 90%, at least 95%, at least 96%, at least 97%, at least 98%, at least 99% or 100% sequence identity to the sequence provided in NP_001997.5, NM_002006.6, NP_001348594.1, or NP_032032.1.

Granulocyte-macrophage colony-stimulating factor (GM-CSF): (e.g., OMIM 138960) A monomeric glycoprotein secreted by macrophages, T cells, mast cells, natural killer cells, endothelial cells and fibroblasts that functions as a cytokine. The pharmaceutical analogs of naturally occurring GM-CSF are called sargramostim and molgramostim. GM-CSF facilitates myeloid stem cell differentiation and can be supplemented at an injury site to increase the efficacy of tissue repair. The immune functions of GM-CSF depend on its targeted presentation during the inflammatory stage of the regenerative cascade, but current protein delivery methods rely on administering supraphysiological doses that act over short periods of time and may cause off-target effects. Thus, GM-CSF affibodies can be used to control the release of GM-CSF and manipulate the immune response or increase angiogenesis, for example to treat a wound or vascular disease. Exemplary GM-CSF sequences can be found in the GenBank® database (e.g., Accession Nos. NP_000749.2; NP_446304.1, NP_999283.1, and M13207.1). In some examples, a GM-CSF protein or coding sequence has at least 80%, at least 90%, at least 95%, at least 96%, at least 97%, at least 98%, at least 99% or 100% sequence identity to the sequence provided in NP_000749.2; NP_446304.1, NP_999283.1, or M13207.1.

Interleukin 4 (IL-4): (e.g., OMIM 147780) A cytokine that induces differentiation of naive helper T cells (Th0 cells) to Th2 cells. Upon activation by IL-4, Th2 cells subsequently produce additional IL-4 in a positive feedback loop. IL-4 is produced primarily by mast cells, Th2 cells, eosinophils and basophils. Thus, IL-4 affibodies can be used to control the release of IL-4 and regulate the immune system, for example reduce inflammation to treat a wound. Exemplary IL-4 sequences can be found in the GenBank® database (e.g., Accession Nos. CAP72493.1, AM937235.1, AAH27514.1 and AAA31055.1). In some examples, an IL-4 protein or coding sequence has at least 80%, at least 90%, at least 95%, at least 96%, at least 97%, at least 98%, at least 99% or 100% sequence identity to the sequence provided in CAP72493.1, AM937235.1, AAH27514.1 or AAA31055.1.

Isolated: An “isolated” biological component, such as a nucleic acid, protein (including affibodies) or organelle, has been substantially separated or purified away from other biological components in the environment (such as a cell) in which the component occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Linker: A peptide sequence that joins two protein domains together, permitting the two protein domains to retain their respective functions, such as specific affinity towards a target. A linker can provide flexibility, stability, and proper spatial orientation to ensure the correct folding and function of the protein domains. Linkers include flexible, rigid, and hybrid linkers, which has both a flexible and rigid segments. Linkers may include repeating units, and the length of the linkers can be adjusted by changing the copy number of the unit to achieve an optimal distance between domains. Flexible linkers are generally rich in small or polar amino acids such as Gly and Ser to provide good flexibility and solubility. In some examples, a flexible linker includes (GGGGS)ₙ, with n=1-6 (SEQ ID NO: 120). Rigid linkers exhibit relatively stiff structures by adopting α-helical structures or by containing multiple Pro residues. Under many circumstances, they separate the functional domains more efficiently than the flexible linkers. In some examples, a rigid linker includes repeating units of EAAAK, such as (EAAAK)ₙ, with n=1-6 (SEQ ID NO: 121), and A(EAAAK)ₙA, with n=2-5 (SEQ ID NO: 122). In some examples, a rigid linker includes Pro-rich sequence, (XP)ₙ, with X designating any amino acid, preferably Ala, Lys, or Glu and n=2-12 (SEQ ID NO: 123). Hybrid linkers have at least one flexible segment (e.g., a segment rich in glycine) and at least one rigid segment (e.g., a segment rich in alanine). In some examples, a hybrid linker includes (GGGGS)ₙ(EAAAK)ₘ, with n=1-6, and m=1-6 (where n and m are independently selected from 1 to 6) (SEQ ID NO: 124). In some examples, a hybrid linker includes (GGG)ₙ(AAAA)ₘ, with n=1-6, and m=1-6 (where n and m are independently selected from 1 to 6) (SEQ ID NO: 125). In some examples, a linker is about 5 to about 12 (such as 5, 6, 7, 8, 9, 10, 11, 12) amino acids in length. Particular examples are provided in SEQ ID NOs: 80-85. In some aspects, a linker includes glycine and serine resides. In some aspects, a linker includes glycine and alanine resides. In some aspects, a linker is short and flexible, such as SEQ ID NO: 80. In some aspects, a linker is long and flexible, such as SEQ ID NO: 81. In some aspects, a linker is rigid and helical, such as SEQ ID NO: 82 or SEQ ID NO: 83. In some aspects, a linker is a hybrid linker, such as SEQ ID NO: 84 or SEQ ID NO: 85.

Localization domain: Referring to a protein domain that binds specifically to a structural bone component, thereby localizing the protein comprising the domain to a material (such as a medical material) containing the structural bone component. In some examples, a localization domain has a K_{D} of less than 1 micromolar (μM) for the corresponding structural bone component. In some examples, a localization domain has a K_{D} of between 1 nanomolar (nM) to 1 M for the corresponding structural bone component, such as from 10⁻⁹ M to 10⁻⁸ M, from 10⁻⁹ M to 10⁻⁷ M, from 10⁻⁹ M to 10⁻⁶ M, from 10⁻⁸ M to 10⁻⁷ M, from 10⁻⁸ M to 10⁻⁶ M, or from 10⁻⁷ M to 10⁻⁶ M. In some examples, a localization domain specifically binds to collagen with a K_{D} of 1 nM to 1 M, such as from 10⁻⁹ M to 10⁻⁸ M, from 10⁻⁹ M to 10⁻⁷ M, from 10⁻⁹ M to 10⁻⁶ M, from 10⁻⁸ M to 10⁻⁷ M, from 10⁻⁸ M to 10⁻⁶ M, or from 10⁻⁷ M to 10⁻⁶ M. In some examples, a localization domain specifically binds to hydroxyapatite (HA) with a K_{D} of 1 nM to 1 μM, such as from 10⁻⁹ M to 10⁻⁸ M, from 10⁻⁹ M to 10⁻⁷ M, from 10⁻⁹ M to 10⁻⁶ M, from 10⁻⁸ M to 10⁻⁷ M, from 10⁻⁸ M to 10⁻⁶ M, or from 10⁻⁷ M to 10⁻⁶ M.

Natural: Not synthetic, such as isolated from a natural source, or produced by a naturally existing organism.

Platelet-derived growth factor (PDGF): Growth factors that regulate cell growth and division. PDGF plays a significant role in blood vessel formation, the growth of blood vessels from already-existing blood vessel tissue, mitogenesis, e.g., proliferation, of mesenchymal cells such as fibroblasts, osteoblasts, tenocytes, vascular smooth muscle cells and mesenchymal stem cells as well as chemotaxis, the directed migration, of mesenchymal cells. Platelet-derived growth factor is a dimeric glycoprotein that can be composed of two A subunits (PDGF-AA), two B subunits (PDGF-BB), or one of each (PDGF-AB). In one example PDGF is PDFG-BB (e.g., OMIM 190040). Thus, PDGF affibodies can be used to control the release of PDGF and increase angiogenesis, for example to treat a wound. Exemplary PDGF-BB sequences can be found in the GenBank® database (e.g., Accession Nos. CAA45383.1, X63966.1, and SM94286.1). In some examples, a PDGF-BB protein or coding sequence has at least 80%, at least 90%, at least 95%, at least 96%, at least 97%, at least 98%, at least 99% or 100% sequence identity to the sequence provided in CAA45383.1, X63966.1, or SM94286.1.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed compositions. Exemplary pharmaceutically and physiologically acceptable fluids includes water, physiological saline, balanced salt solutions, aqueous dextrose, glycerol or the like. In particular embodiments, suitable for administration to a subject the carrier may be sterile.

Peptide or Polypeptide: A polymer in which the monomers are amino acid residues that are joined together through amide bonds. When the amino acids are alpha-amino acids, either the L-optical isomer or the D-optical isomer can be used. The term polypeptide or protein as used herein encompasses any amino acid sequence and includes modified sequences such as glycoproteins. The term polypeptide is specifically intended to those that are recombinantly or synthetically produced. A peptide has an amino (N) terminus and a carboxy (C) terminus. The N- or C-terminus of a polypeptide can be joined (for example, by peptide bond) to heterologous amino acids, such as a peptide tag, or a cysteine (or other, such as Lys, Tyr, Try, or Phe) residue in the context of a linker for conjugation chemistry. The phrase “functional fragment(s) of a polypeptide” refers to all fragments of a polypeptide that retain an activity, or a measurable portion of an activity, of the polypeptide from which the fragment is derived.

Purified: The term purified does not require absolute purity; rather, it is intended as a relative term. Thus, for example, a purified dual-affinity fusion protein preparation is one in which the dual-affinity fusion protein is more enriched than the dual-affinity fusion protein is in its environment within a cell or other mixture. In one aspect, a preparation is purified such that the dual-affinity fusion protein represents at least 50% of the total protein content of the preparation. Substantial purification denotes purification from other proteins or cellular components. A substantially purified dual-affinity fusion protein is at least 60%, 70%, 80%, 90%, 95%, 98%, 99%, 99.9% or 99/99% pure. Thus, in one specific, non-limiting example, a substantially purified dual-affinity fusion protein is 90% free of other proteins or cellular components.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of an affibody, localization domain, or dual-affinity fusion protein provided herein are typically characterized by possession of at least about 80%, for example at least about 85%, 90%, 95%, 96%, 97%, 98% or 99% sequence identity counted over the full-length alignment with the amino acid sequence of the affibody, localization domain, or dual-affinity fusion protein using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Affibodies, localization domains, and dual-affinity fusion proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10⁻²⁰ amino acids and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that variants with similar activity could be obtained that fall outside of the ranges provided.

Structural bone component: Components that provide structure to natural bone and synthetic bone (e.g., synthetic graft, filler, scaffold, sponge, etc.), including collagen (e.g., Type I, Type II, Type III, or Type V collagen), a calcium phosphate, and a calcium sulphate. In some aspects, the calcium phosphate is hydroxyapatite (HA), β-tricalcium phosphate (β-TCP), α-tricalcium phosphate (α-TCP), calcium deficient hydroxyapatite (CDHA), tetracalcium phosphate (TTCP), amorphous calcium phosphate (ACP), dicalcium phosphate dihydrate (DCPD), or dicalcium phosphate anhydrous (DCPA).

Subject or patient: A term that includes human and non-human mammals. In one example, the subject is a human or veterinary subject, such as a mouse, rat, dog, cat, or non-human primate. In some examples, the subject is a mammal (such as a human) who has a bone injury (such as a fracture, such as a non-union fracture, or due to cancer, osteoporosis, or osteoarthritis), wound (including wounds that damage vascular networks), a vascular disease (e.g., diabetic ulcer, critical limb ischemia, peripheral artery disease, cerebrovascular diseases including stroke, migraine and other headache disorders), or neurological injury or disorder (e.g., paralysis, acute spinal cord injury, stroke, traumatic brain injury, other head trauma, epilepsy, Alzheimer's disease and other dementias, ALS, multiple sclerosis, Parkinson's disease).

Synthetic: Produced by artificial means in a laboratory, for example a synthetic nucleic acid or protein (for example, an affibody) can be chemically synthesized in a laboratory.

Therapeutic protein: A protein that can exert a therapeutic effect in a subject in need thereof, including growth factors such as bone growth factors (e.g., BMPs, FGFs, PDGFs, and VEGFs), and hematopoietic growth factors (e.g., GM-CSF); cytokines such as interleukins (e.g., IL-4). In some aspects, the therapeutic effect is promoting tissue regeneration (e.g., bone growth, wound healing, etc.), promoting angiogenesis, promoting cell infiltration, or modulating immune response in the environment of an injured or diseased site.

Treating a disease: Includes inhibiting or preventing the partial or full development or progression of a disease, for example in a person who is known to have a predisposition to a disease. Furthermore, treating a disease refers to a therapeutic intervention that ameliorates at least one sign or symptom of a disease or pathological condition, or interferes with a pathophysiological process, after the disease or pathological condition has begun to develop.

Under conditions sufficient for: A phrase that is used to describe any environment that permits the desired activity. In one example, includes administering a therapeutically effective amount of a composition as provided herein sufficient to enable the desired activity.

Vasculature: The network of blood vessels connecting the heart with all other organs and tissues in the body. It includes the arteries and arterioles, bringing oxygen-rich blood to the organs and tissues, and the veins and venules carrying deoxygenated blood back to the heart. A “resistance artery” is a blood vessel in the microcirculation that contributes to the creation of resistance to blood flow. Resistance vessels are innervated by autonomic nerves, and constrict and dilate in response to circulating hormones. Resistance in small arteries (lumen diameter<350 micrometers) and arterioles (lumen diameter<100 micrometers) accounts for 45-50% of total peripheral resistance.

Vascular endothelial growth factor (VEGF): A signal protein produced by many cells that stimulates the formation of blood vessels. VEGF is a sub-family of growth factors, the platelet-derived growth factor family of cystine-knot growth factors. They are signaling proteins involved in vasculogenesis (the de novo formation of the embryonic circulatory system) and angiogenesis (the growth of blood vessels from pre-existing vasculature). In one example VEGF is VEGF₁₆S (also known as neuropilin, e.g., OMIM 602069), a transmembrane protein involved in vasculogenesis and angiogenesis. Thus, VEGF affibodies can be used to control the release of VEGF and increase angiogenesis, for example to treat a wound or vascular disease. Exemplary VEGF₁₆₅ sequences can be found in the GenBank® database (e.g., Accession Nos. AAC12921, AAC51759.1, AF016050.1, AAC53345.1 and BAA08789.1). In some examples, a VEGF₁₆₅ protein or coding sequence has at least 80%, at least 90%, at least 95%, at least 96%, at least 97%, at least 98%, at least 99% or 100% sequence identity to the sequence provided in GenBank Accession No. AAC12921, AAC51759.1, AF016050.1, AAC53345.1 or BAA08789.1.

Wound: An injury or damage to living tissue.

Wound repair: The process of replacing damaged or missing cellular structures or tissue layers. Wound repair (or wound healing) is characterized by the steps of hemostasis (blood clotting), inflammation, proliferation (growth of new tissues) and remodeling.

### Overview

Directed evolution was used to generate affibodies, a class of small, α-helical, antibody-mimetic proteins that can be engineered to bind to a target protein.^{42,43} Affibodies are currently being tested clinically and preclinically as targeting agents for HER2⁺ breast cancer cells,^{44,45} and for the detection of other biological markers, such as CD69 cell markers for early detection of activated immune cells⁴⁶ and vascular endothelial growth factor receptor-2 (VEGFR2) expression for analyzing angiogenesis signaling pathways.⁴⁷ Moreover, affibodies have also been used to tune the release of fibroblast growth factor-2 (FGF-2),⁴⁰ insulin-like growth factor-1 (IGF-1), and pigment epithelium-derived factor (PEDF).³⁹ Their clinical benefit is derived from their relatively stable structure under physiological conditions, the diversity of proteins to which they can bind, and the ability to modify their binding affinity by changing 13 to 17 amino acids at the binding interface between the affibody and target protein.^{43,48} However, the tunability of affibody affinity is underutilized, as affibody affinity has thus far only been maximized for targeting endogenous protein species without considering the use of multiple affibodies displaying a range of moderate affinities for tuning the delivery rates of exogenous proteins. While typical affinity binders generated via directed evolution target strong interactions with equilibrium dissociation constants in the picomolar range,⁴⁹ affibodies with moderate affinity interactions with equilibrium dissociation constants in the nanomolar range enable controlled protein release. Disclosed dual-affinity fusion proteins that include an affibody, each fusion protein having an affibody with a different affinity for a therapeutic protein, can be tuned to release the therapeutic protein at specific rates.

BMP-2-specific affibodies were identified with a range of affinities for BMP-2 from a yeast surface display library containing 10⁸ affibody variants. In addition to BMP-2 affibodies, using similar methods, affibodies for vascular endothelial growth factor (VEGF), fibroblast growth factor 2 (FGF-2), platelet-derived growth factor (PDGF), granulocyte-macrophage colony-stimulating factor (GM-CSF), and interleukin-4 (IL-4) were identified and tested. These affibodies were incorporated into a dual-affinity fusion protein to control BMP-2, VEGF, FGF-2, PDGF, GM-CSF, and/or IL-4 release. The dual-affinity fusion protein also included a localization domain that specifically binds to a component of a medical material typically used in treatment of wound, or a bone or cartilage injury or disease, such as an implantable collagen sponge. Thus, one or more disclosed dual-affinity fusion proteins can be loaded onto or into the medical material to control release of BMP-2, VEGF, FGF-2, PDGF, GM-CSF, and/or IL-4, when the medical material is used. BMP-2-specific affibodies were identified that minimally interact with other proteins involved in the tissue healing cascade and have significantly different equilibrium dissociation constants to tune the release kinetics of BMP-2. In some examples, these BMP-2-specific affibodies did not interact with several other key proteins in the bone healing cascade, e.g., VEGF, IL-4, or GM-CSF. Computational modeling was used to predict the binding interface between the affibodies and BMP-2, revealing that the high-affinity affibodies may bind BMP-2 at a different interface than the low-affinity affibodies. BMP-2 bound to affibodies demonstrated diminished osteogenic properties in vitro.

The ability to identify affibodies and fusion proteins that impact protein bioactivity permits spatiotemporal control over protein activity. The affibodies and fusion proteins disclosed herein provide the specificity necessary to control protein release in vivo more precisely, which can improve clinical protein delivery strategies.

A composition can include one or more dual-affinity fusion proteins each including an affibody domain specific for a single therapeutic protein, wherein each dual-affinity fusion protein has a particular K_{D} with respect to the therapeutic protein. The composition may include the dual-affinity fusion proteins bound to their corresponding therapeutic protein. Due to the differences in K_{D}'s, the rate of release of the therapeutic protein from the fusion proteins will vary depending on the K_{D} of the affibody. For example, a weak affinity affibody domain (e.g., one with a higher K_{D}) will release its therapeutic protein from the fusion protein more readily than a medium- or strong affinity affibody domain (e.g., one with a lower K_{D}). In some examples, the K_{D} of a high affinity affibody domain is at least 2-fold, at least 3-fold, at least 4-fold, at least 5-fold, at least 6-fold, at least 7-fold, at least 8-fold, at least 9-fold, or at least 10-fold lower than the K_{D} of a moderate affinity affibody domain or a low affinity affibody domain. In some examples, the K_{D} of a low affinity affibody domain is at least 2-fold, at least 3-fold, at least 4-fold, at least 5-fold, at least 6-fold, at least 7-fold, at least 8-fold, at least 9-fold, or at least 10-fold higher than the K_{D} of a moderate affinity affibody domain or a high affinity affibody domain. In some examples, the K_{D} of a moderate affinity affibody domain is at least 2-fold, at least 3-fold, at least 4-fold, at least 5-fold, at least 6-fold, at least 7-fold, at least 8-fold, at least 9-fold, or at least 10-fold higher than the K_{D} of a high affinity affibody domain or at least 2-fold, at least 3-fold, at least 4-fold, at least 5-fold, at least 6-fold, at least 7-fold, at least 8-fold, at least 9-fold, or at least 10-fold lower than the K_{D} of a low affinity affibody domain.

Due to the differences in K_{D}'s of each unique affibody domain, the rate of release of the therapeutic proteins from the fusion proteins will vary depending on the K_{D} of the affibody. For example, a weak affinity affibody will release its protein from the fusion protein more readily than a medium- or strong-affinity affibody domain.

One skilled in the art will appreciate that a composition can include (a) one or more fusion proteins each including an affibody domain specific for one therapeutic protein, wherein each unique fusion protein has a specific K_{D} for the protein, or (b) one or more fusion proteins specific for one or more therapeutic proteins, wherein each unique fusion protein has a specific K_{D} for its corresponding therapeutic protein.

Large bone defects often fail to heal, resulting in fracture nonunion. Frozen or freeze-dried bone allografts are convenient sources of donor bone that provide structural and osteoconductive support to bridge large bone defects. However, close to 60% of bone allografts fail within 10 years after implantation due to poor integration with host tissue. In contrast, live autologous bone grafts, which contain viable cells that secrete paracrine factors, typically integrate robustly, recapitulating the natural bone healing cascade. Coordinated immune responses are necessary to stimulate bone graft integration into host tissue; however, critical cytokines missing in processed allografts may hinder the appropriate inflammatory response needed for repair. GM-CSF and IL-4 mediate the acute inflammatory response to injury and activate macrophages to stimulate bone remodeling at the graft-host interface. BMP-2 is an osteoinductive protein that can stimulate robust bone regeneration. Collectively, BMP-2, GM-CSF, and IL-4 play roles in graft integration, by stimulating mineralization to promote osseointegration and the inflammatory response and extracellular matrix (ECM) deposition to mediate remodeling of the graft, respectively. However, uncontrolled release of BMP-2, IL-4, and GM-CSF may have detrimental effects of bone allograft integration. High BMP-2 doses cause heterotopic ossification in human patients, while excess IL-4 can cause fibrosis and delay healing. Furthermore, simultaneous delivery of GM-CSF and IL-4 without spatiotemporal control may result in suboptimal dendritic cell phenotypes that extend the pro-inflammatory phase of healing. Thus, combining IL-4, GM-CSF and BMP-2 can be used to enhance graft integration, for example using a method that enables sustained, local delivery of cytokines within bone allografts to modulate the immune response to the implant and improve allograft integration.

The fusion proteins provided herein enhance bone allograft integration, by affinity-controlled and allograft-localized delivery of immunomodulatory and osteogenic proteins. Unlike traditional methods of protein localization, such as covalent tethering or encapsulation within a polymer matrix, the fusion proteins provided herein maintain protein bioactivity and provide predictable, independent control over protein retention/release using orthogonal affinity interactions.

### Dual-Affinity Fusion Proteins and Compositions

The present disclosure provides dual-affinity fusion proteins. Such fusion proteins contain an affibody sequence (used interchangeably with “affibody domain”), a linker, and a localization domain, wherein the affibody sequence is specific for a therapeutic protein, and the localization domain is specific for a structural bone component (which can be synthetic or natural). The present disclosure also provides compositions including one or more of the dual-affinity fusion proteins. In some examples, the compositions further include one or more therapeutic proteins, and/or a medical material including the structural bone component, wherein each of the fusion proteins includes an affibody domain specific for one of the therapeutic proteins. In some examples, the one or more fusion proteins are bound to the one or more therapeutic proteins non-covalently. In some examples, the one or more fusion proteins are bound to the structural bone component contained in the medical material non-covalently. For example, if one or more fusion proteins each include a different affibody domain specific for BMP-2, the composition can include BMP-2 non-covalently bound to the one or more fusion proteins through the different BMP-2-specific affibody domains. In some examples, the composition further includes the medical material, and the one or more fusion proteins that are non-covalently bound with the one or more therapeutic proteins are also non-covalently bound with the medical material, through the specific affinity interaction between the localization domain of each fusion protein and the medical material. Such compositions can further include a pharmaceutically acceptable carrier, such as water or saline or a buffer. In some examples, such compositions can be used to control the release of one or more therapeutic proteins, for example when using the medical material to treat a wound, or a bone or cartilage injury or disease.

In some examples, the dual-affinity fusion protein-containing composition includes at least one of bone morphogenetic protein 2 (BMP-2) protein, vascular endothelial growth factor (VEGF) protein (such as VEGF₁₆₅), fibroblast growth factor 2 (FGF-2) protein, platelet-derived growth factor (PDGF) protein (such as PDGF-BB), granulocyte-macrophage colony-stimulating factor (GM-CSF) protein, interleukin-4 (IL-4) protein, and glial derived neurotrophic factor (GDNF) protein, and corresponding fusion proteins including affibody domains specific for BMP-2, VEGF, FGF-2, PDGF, GM-CSF, IL-4, and/or GDNF.

In some examples, the composition includes one or more fusion proteins each including a unique affibody domain specific for a single protein (such as one of BMP-2, VEGF, FGF-2, PDGF, GM-CSF, IL-4, or GDNF). In some examples, the composition includes one fusion protein including a unique affibody domain specific for a single protein (such as one of BMP-2, VEGF, FGF-2, PDGF, GM-CSF, IL-4, or GDNF). In some examples, the composition includes at least one fusion proteins each including a unique affibody domain (such as at least 2, at least 3, at least 4, at least 5 or at least 10, such as 2, 3, 4, 5, 6, 7, 8, 9, or 10 fusion proteins each including a unique affibody domain) specific for a single protein (such as one of f BMP-2, VEGF, FGF-2, PDGF, GM-CSF, IL-4, or GDNF), wherein each unique affibody domain has a different K_{D} for the protein. For example, the composition can include a fusion protein including a weak affinity affibody domain (e.g., one with a higher K_{D}), and a fusion protein including a strong affinity affibody domain (e.g., one with a lower K_{D}) for BMP-2, VEGF, FGF-2, PDGF, GM-CSF, IL-4, or GDNF. In one example, the composition includes a fusion protein including a weak affinity affibody domain (e.g., one with a higher K_{D}), a fusion protein including a medium affinity affibody domain (e.g., one with a K_{D} lower than that of the weak affinity antibody), and a fusion protein including a strong affinity affibody domain (e.g., one with a K_{D} lower than that of the weak or medium-affinity affibody domain) for BMP-2, VEGF, FGF-2, PDGF, GM-CSF, IL-4, or GDNF.

In some examples, the composition includes two or more therapeutic proteins (or 3 or more, 4 or more, 5 or more, 6 or more, or 7 proteins) selected from BMP-2, VEGF, FGF-2, PDGF, GM-CSF, IL-4, and GDNF, and fusion proteins with corresponding affibody domains. In some examples, the composition includes two or more therapeutic proteins (such as 2, 3, 4, 5, 6, or 7 proteins) selected from BMP-2, VEGF, FGF-2, PDGF, GM-CSF, IL-4, and GDNF, and one corresponding fusion protein (with a unique affibody domain) for each therapeutic protein. In some examples, the composition includes two or more therapeutic proteins (such as 2, 3, 4, 5, 6, or 7 proteins) selected from BMP-2, VEGF, FGF-2, PDGF, GM-CSF, IL-4, and GDNF, and two or more corresponding fusion proteins (each with a unique affibody domain) for each therapeutic protein (such as at least 3, at least 4, at least 5, or at least 10 fusion proteins (each with a unique affibody domains) for each protein, such as 2, 3, 4, or 5 fusion proteins for each protein). If 2 or more fusion proteins are present for the same therapeutic protein, each fusion protein includes a unique affibody domain that has a different K_{D} for the protein. For example, the composition can include a fusion protein with a weak affinity affibody domain (e.g., one with a higher K_{D}), and a fusion protein with a strong affinity affibody (e.g., one with a lower K_{D}) for BMP-2, VEGF, FGF-2, PDGF, GM-CSF, IL-4, and/or GDNF. In one example, the composition includes a fusion protein with a weak affinity affibody domain (e.g., one with a higher K_{D}), a fusion protein with a medium affinity affibody domain (e.g., one with a K_{D} lower than that of the weak affinity antibody domain), and a fusion protein with a strong affinity affibody domain (e.g., one with a K_{D} lower than the weak or medium-affinity affibody domain) for BMP-2, VEGF, FGF-2, PDGF, GM-CSF, IL-4, and/or GDNF.

In some examples, the composition includes the following therapeutic proteins and one or more fusion proteins with affibody domains specific for the therapeutic proteins: a) VEGF, FGF2, and PDGF-BB; b) GM-CSF; c) GDNF; d) VEGF, FGF2, PDGF-BB, and BMP-2; e) GM-CSF and IL-4; f) GM-CSF, IL-4 and MCP-1; g) BMP-2 and IL-4; h) BMP-2; i) GM-CSF, IL-4, and BMP-2, or j) PDGF-BB and VEGF.

The compositions can include additional therapeutic proteins and fusion proteins, such as collagen I, collagen III, and/or monocyte chemoattractant protein-1 (MCP-1), and one or more fusion proteins with corresponding affibody domains. In some examples the composition further includes one or more additional chemoattractant proteins (e.g., MCP-1, SDF-1a) and fusion proteins, cytokine proteins (e.g., IL-10) and fusion proteins, immunomodulatory proteins (e.g., IL-10, MCP-1, G-CSF) and fusion proteins, and/or morphogen proteins (e.g., NGF, NT-3, BDNF) and fusion proteins.

The composition can include at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 20, 25, 30, 35, 40, 45, 50, 75, 100 or more) different fusion proteins each with an affibody domain specific for a single therapeutic protein. In such examples, each fusion protein with a unique affibody domain can have a unique affinity or K_{D} for the therapeutic protein, such as at least one with a low K_{D}/strong affinity (e.g., K_{D} about 10⁻⁹-10⁻⁸ M), at least one with a medium K_{D}/medium affinity (e.g., K_{D} about 10⁻⁷-10⁻⁶ M) and at least one with a higher K_{D}/weak affinity (e.g., K_{D} about 10⁻⁵-10⁻³ M). In some examples, each fusion protein with a unique affibody has a unique affinity or K_{D} for the therapeutic protein, such as at least one with a low K_{D}/strong affinity (e.g., K_{D} about 10⁻⁹-10⁻⁸ M) and at least one with a higher K_{D}/weak affinity (e.g., K_{D} about 10⁻⁵-10⁻³ M). In some examples a low K_{D}/strong affinity fusion protein has a K_{D} (with respect to the therapeutic protein) that is at least 2-fold, at least 3-fold, at least 4-fold, at least 5-fold, at least 6-fold, at least 7-fold, at least 8-fold, at least 9-fold, or at least 10-fold lower than a medium K_{D}/medium affinity fusion protein. In some examples a high K_{D}/low affinity fusion protein has a K_{D} (with respect to the therapeutic protein) that is at least 2-fold, at least 3-fold, at least 4-fold, at least 5-fold, at least 6-fold, at least 7-fold, at least 8-fold, at least 9-fold, or at least 10-fold higher than a medium K_{D}/medium affinity affibody. In some examples, each fusion protein with a unique affibody domain has a K_{D} for the therapeutic protein that is at least an order of magnitude (e.g., at least about 10-fold) different from the K_{D} of another fusion protein with a unique affibody for the same therapeutic protein. Thus, in some examples, a low K_{D}/strong affinity fusion protein has a K_{D} (with respect to the therapeutic protein) that is at least about 10 times lower than that of a medium K_{D}/medium affinity fusion protein, and a medium K_{D}/medium affinity fusion protein has a K_{D} (with respect to the therapeutic protein) that is at least about 10 times lower than a higher K_{D}/weak affinity affibody.

In some examples, the composition includes at least 1, at least 2, at least 3, at least 4, at least 5, at least 10, at least 15, at least 20, at least 30, at least 40, or at least 50 (such as 1, 2, 3, 4, 5, 6, 7, 8, 9, 10, 15, 20, 25, 30, 35, 40, 45, 50, 75, 100 or more) different/unique fusion proteins with different/unique affibody domains, wherein each unique fusion protein is specific for a single therapeutic protein. In some examples, combinations are used (e.g., two or more fusion proteins specific for protein 1, and two or more fusion proteins specific for protein 2, etc.). In some examples, where two or more unique fusion proteins are present that are specific for the same therapeutic protein, each unique fusion protein can have a distinct K_{D} (with respect to the therapeutic protein), such as one with a higher and another with a lower K_{D} (such as at least 2-fold, at least 3-fold, at least 5-fold, or at least 10-fold difference).

In some aspects, the compositions include a medical material used to treat a wound and/or a bone or cartilage injury or disease. Medical material is material for or intended for medical use. In some aspects, medical material is biocompatible (e.g., compatible with living tissues, without causing uncontrollable rejection). In some aspects, medical material is biodegradable (e.g., will dissolve over time in the subject). In some aspects, the one or more fusion proteins bind specifically to the medical material, through non-covalent affinity interactions between the localization domain of the fusion protein and the structural bone component contained by the medical material. The structural bone component recognized by the localization domain can be natural (such as components contained in bone autograft or allograft) or synthetic (such as components included in artificial medical material that mimics the compositions of bone or cartilage, such as collagen scaffold, collagen sponge, bone void filler, bone implant, etc.). In some aspects, the structural bone component recognized by the localization domain is organic (such as collagen). In some aspects, the structural bone component recognized by the localization domain is inorganic (such as a calcium phosphate, a calcium sulphate, etc.). In some examples, the structural bone component is a collagen, such as Type I collagen, Type II collagen, or Type III collagen. In some examples, the structural bone component is a calcium phosphate, such as hydroxyapatite (HA), β-tricalcium phosphate (β-TCP), α-tricalcium phosphate (α-TCP), calcium deficient hydroxyapatite (CDHA), tetracalcium phosphate (TTCP), amorphous calcium phosphate (ACP), dicalcium phosphate dihydrate (DCPD), or dicalcium phosphate anhydrous (DCPA).

Linker sequences are used to join the localization domain and the affibody domain together. In some examples, linker sequences are designed to separate the localization domain and the affibody domain so that neither of them occludes or interferes with the binding of the other. In some examples, the linker is a flexible linker, a rigid linker, or a hybrid linker including a flexible and a rigid segment. In one aspects, the linker is a rigid linker. Flexible linkers are generally rich in small or polar amino acids such as Gly and Ser to provide good flexibility and solubility. In some examples, a flexible linker includes (GGGGS)ₙ, with n=1-6 (SEQ ID NO: 120). Rigid linkers exhibit relatively stiff structures by adopting α-helical structures or by containing multiple Pro residues. In some examples, a rigid linker includes repeating units of EAAAK, such as (EAAAK)ₙ, with n=1-6 (SEQ ID NO: 121), and A(EAAAK)ₙA, with n=2-5 (SEQ ID NO: 122). In some examples, a rigid linker includes Pro-rich sequence, (XP)ₙ, with X designating any amino acid, preferably Ala, Lys, or Glu and n=2-12 (SEQ ID NO: 123). Hybrid linkers can include at least one flexible segment (e.g., a segment rich in glycine) and at least one rigid segment (e.g., a segment rich in alanine). In some examples, a hybrid linker includes (GGGGS)ₙ(EAAAK)ₘ, with n=1-6, and m=1-6 (where n and m are independently selected from 1 to 6) (SEQ ID NO: 124). In some examples, a hybrid linker includes (GGG)ₙ(AAAA)ₘ, with n=1-6, and m=1-6 (where n and m are independently selected from 1 to 6) (SEQ ID NO: 125). In some examples, a linker is about 5 to about 12 (such as 5, 6, 7, 8, 9, 10, 11, 12) amino acids in length. Particular examples are provided in SEQ ID NOs: 80-85. In some examples, a linker includes glycine and serine resides. In some examples, a linker includes glycine and alanine resides. In some examples, a linker is short and flexible, such as SEQ ID NO: 80. In some examples, a linker is long and flexible, such as SEQ ID NO: 81. In some examples, a linker is rigid and helical, such as SEQ ID NO: 82 or SEQ ID NO: 83. In some examples, a linker is a hybrid linker, such as SEQ ID NO: 84 or SEQ ID NO: 85.

Exemplary sequences for affibody domains, linkers, localization domains, and fusion proteins encompassed by the disclosure are provided in Table 1 and Table 2, and these sequences and functional variants thereof can be used in the compositions and methods provided herein. In some examples, an affibody domain in a fusion protein provided herein includes or consists of any of SEQ ID NOs: 1-68 and 71-74, or has at least 90%, at least 95%, at least 96%, at least 97%, at least 98%, at least 99%, or 100% sequence identity to any of SEQ ID NOs: 1-68 and 71-74. In some examples, an affibody domain in a fusion protein provided herein includes or consists of any of SEQ ID NOs: 1-68 and 71-74 but without the initial A (alanine) of these sequences, or has at least 90%, at least 95%, at least 96%, at least 97%, at least 98%, at least 99%, or 100% sequence identity to any of SEQ ID NOs: 1-68 and 71-74 without the initial A. In some examples, the affibody includes an optional His-tag (SEQ ID NO: 86) at its N- or C-terminal. In some examples, the affibody domain is 56-80 amino acids, such as 56-65, 57-58, or 56, 57, 58, 59, 60, 61, 62, 63, 64, 65, 66, 67, 68, 69 or 70 amino acids in length. In some examples, the affibody domain has 1, 2, 3, 4, 5 or 6 conservative amino acid substitutions based on any of SEQ ID NOs: 1-68 and 71-74, with or without the initial A. In one example, the one or more affibody domains in one or more fusion proteins include one or more of SEQ ID NOS: 1, 2, 3, 12, 13, 14, 20, 21, 22, 42, 43, 44, 57, 58, 59, 60, 61, 62, 63, 64, 71, 72, 73, and 74, with or without the initial A.

TABLE 1 Exemplary affibody sequences (SEQ ID NO: in parentheses).

K_{D}(nM)

BMP-2 Affibodies

| | | |
|---|---|---|
| High | AEAKYYKEVSSAATQIRYLPNLTAFQKAAFYAALLDDPS | 10.7 |
| Affinity | QSSELLSEAKKLNDSQAPK (1) | |
| (A1-2) | | |
| Moderate | AEAKYAKEQFNAYVVIFYLPNLTASQKAAFVDALSNDPS | 10.4 |
| Affinity | QSSELLSEAKKLNDSQAPK (2) | |
| (A2-2) | | |
| Low | AEAKYYKEGDNAYNVIYGLPNLTRPQRLAFIVALENDPSQ | 34.8 |
| Affinity 1) | (B4-SSELLSEAKKLNDSQAPK (3) | |
| A1-1 | AEAKYNKEVTAAANSIWVLPNLTGDQKAAFFEALLDDPS | |
| | QSSELLSEAKKLNDSQAPK (4) | |
| A1-3 | AEAKYTKEGFDAYDVIDNLPNLTLDQRNAFVYALENDPS | |
| | QSSELLSEAKKLNDSQAPK (5) | |
| A2-1 | AEAKYYKEWLDADMSIRSLPNLTGYQIRAFIAALGNDPSQ | |
| | SSELLSEAKKLNDSQAPK (6) | |
| A2-3 | AEAKYYKERRAAAVVIFYLPNLTRVQKGAFIEALDDDPSQ | |
| | SSELLSEAKKLNDSQAPK (7) | |
| A3-1 | AEAKYAKERLNAIYVINDLPNLTQGQRVAFARALYNDPS | |
| | QSSELLSEAKKLNDSQAPK (8) | |
| A3-2 | AEAKYAKEQFNAYVVIFYLPNLTASQKAAFVDALSNDPS | |
| | QSSELLSEAKKLNDSQAPK (9) | |
| B3-3 | AEAKYYKEWVNAYDQIRVLPNLTRFQRLAFYRALYNDPS | |
| | QSSELLSEAKKLNDSQAPK (10) | |
| B4-2 | AEAKYYKEWLDADMSIRSLPNLTGYQIRAFIAALGNDPSQ | |
| | SSELLSEAKKLNDSQAPK (11) | |

GM-CSF Affibodies

| | | |
|---|---|---|
| GM3-C4 | AEAKYTKELFNAVGEITALPNLTRYHLY AFYYALLNDPSQ | 441.4 |
| (High | SSELLSEAKKLNDSQAPK (12) | |
| Affinity) | | |
| GM4-C4 | AEAKYNKEWFAADLSIGFLPNLTLDQLYAFVFALYDDPS | 971.0 |
| (Mid | QSSELLSEAKKLNDSQAPK (13) | |
| affinity) | | |
| GM4-C3 | AEAKYAKEGLNAYLSIRWLPNLTGDQMYAFISALLDDPS | 3783 |
| (Low | QSSELLSEAKKLNDSQAPK (14) | |
| Affinity) | | |
| GM1-C3 | AEAKYTKEGFNAYDEIDNLPNLTLDQRNAFVYALFNDPS | |
| | QSSELLSEAKKLNDSQAPK (15) | |
| GM2-C4 | AEAKYTKELFNAVGEITALPNLTRYHLYAFYYALLNDPSQ | |
| | SSELLSEAKKLNDSQAPK (16) | |
| GM3-C1 | AEAKYNKEVGTANFEIVLLPNLTLYQMLAFIKALVNDPSQ | 296.7 |
| | SSELLSEAKKLNDSQAPK (17) | |
| GM3-C2 | AEAKYNKEWYNAISVIFYLPNLTGFQRAAFVDALGDDPS | |
| | QSSELLSEAKKLNDSQAPK (18) | |
| GM4-C2 | AEAKYYKEGFYANFVIGALPNLTLVQRAAFYFALLNDPS | 786.7 |
| | QSSELLSEAKKLNDSQAPK (19) | |

VEGF Affibodies

| | | |
|---|---|---|
| TM2 (High | AEAKYYKEGATAYRVIEYLPNLTGAQKAAFIDALYNDPS | 58.3 |
| Affinity) | QSSELLSEAKKLNDSQAPK (20) | |
| LG2 (Mid | AEAKYTKEGFDAYDVIDNLPNLTLDQRNAFVYALENDPS | 307 |
| Affinity) | QSSELLSEAKKLNDSQAPK (21) | |
| BR2 (Low | AEAKYNKEWYDAVFVIGSLPNLTEDQKDAFSDALVDDPS | 6470 |
| Affinity) | QSSELLSEAKKLNDSQAPK (22) | |
| L1 | AEAKYYKEWNAAYVVINGLPNLTRRQREAFVHALVDDP | |
| | SQSSELLSEAKKLNDSQAPK (23) | |
| L3 | AEAKYYKERYAANYSIWVLPNLTLLQRFAFFFALSNDPSQ | |
| | SSELLSEAKKLNDSQAPK (24) | |
| L4 | AEAKYAKELDDAFFEIASLPNLTGFQLHAFAVALGNDPSQ | |
| | SSELLSEAKKLNDSQAPK (25) | |
| L5 | AEAKYNKERDSAYSVIWGLPNLTDSQKAAFGYALYNDPS | |
| | QSSELLSEAKKLNDSQAPK (26) | |
| L7 | AEAKYAKELEAANMVIVDLPNLTHGQKVAFLVALENDPS | |
| | QSSELLSEAKKLNDSQAPK (27) | |
| L8 | AEAKYNKEWYDAILEIGFLPNLTGHQRDAFSDALVDDPS | |
| | QSSELLSEAKKLNDSQAPK (28) | |
| L10 | AEAKYNKEQDSAYSVIWGLPNLTESQKAAFGYALYDDPS | |
| | QSSELLSEAKKLNDSQAPK (29) | |
| BM1 | AEAKYNKEVTAAANSIWVLPNLTGDQKAAFFEALLDDPS | |
| | QSSELLSEAKKLNDSQAPK (30) | |
| BM2 | AEAKYAKEWFYAYHVIYDLPNLTGFQKHAFYLALYDDPS | |
| | QSSELLSEAKKLNDSQAPK (31) | |
| BM3 | AEAKYNKEVTAAANSIWVLPNLTGDQKAAFFEALLDDPS | |
| | QSSELLSEAKKLNDSQAPK (32) | |
| BM7 | AEAKYAKEGATAFGSIPYLPNLTDVQRYAFIVALLDDPSQ | |
| | SSELLSEAKKLNDSQAPK (33) | |
| BR1 | AEAKYTKEWYAAVVQIGYLPNLTAFQRAAFSFALSNDPS | |
| | QSSELLSEAKKLNDSQAPK (34) | |
| BR3 | AEAKYTKERDDASLEIAYLPNLTPYQLMAFFFALSNDPSQ | |
| | SSELLSEAKKLNDSQAPK (35) | |
| BR5 | AEAKYAKEWTNAFVSIVCLPNLTAVQREAFVLALVDDPS | |
| | QSSELLSEAKKLNDSQAPK (36) | |
| BR6 | AEAKYAKEWEDAINEIWCLPNLTEYQRIAFVSALYNDPSQ | |
| | SSELLSEAKKLNDSQAPK (37) | |
| BR7 | AEAKYAKELLNAFDEIYGLPNLTVGQRMAFCDALINDPSQ | |
| | SSELLSEAKKLNDSQAPK (38) | |
| TM4 | AEAKYYKEWYDAFVVIDALPNLTAYQREAFIFALVNDPS | |
| | QSSELLSEAKKLNDSQAPK (39) | |
| TM6 | AEAKYYKEWVDAYLVIDSLPNLTRLQVEAFVFALVNDPS | |
| | QSSELLSEAKKLNDSQAPK (40) | |
| TM7 | AEAKYTKEVDYAACVIAYLPNLTGVQVYAFYRALADDPS | |
| | QSSELLSEAKKLNDSQAPK (41) | |
| D28A (Low | AEAKYNKEWYDAVFVIGSLPNLTEDQKAAFSDALVDDPS | 1835 |
| Affinity) | QSSELLSEAKKLNDSQAPK (71) | |
| D32A (Low | AEAKYNKEWYDAVFVIGSLPNLTEDQKDAFSAALVDDPS | 4186 |
| Affinity) | QSSELLSEAKKLNDSQAPK (72) | |
| D36A (High | AEAKYNKEWYDAVFVIGSLPNLTEDQKDAFSDALVADPS | 109 |
| Affinity) | QSSELLSEAKKLNDSQAPK (73) | |

FGF-2 Affibodies

| | | |
|---|---|---|
| FG2-C1 | AEAKYTKEGSDAFDVIVLLPNLTRDQRDAFLY ALLDDPSQ | 3.08 |
| (High) | SSELLSEAKKLNDSQAPK (42) | |
| FG3-C1 | AEAKYAKEWLSADYVIICLPNLTLDQMVAFYDALENDPS | 121 |
| (Mid) | QSSELLSEAKKLNDSQAPK (43) | |
| FG3-C4 | AEAKYNKEVFDADCSIWYLPNLTRYQISAFQSALDDDPSQ | 4550 |
| (Low) | SSELLSEAKKLNDSQAPK (44) | |
| FG1_C1 | AEAKYTKEGCDAYTEIVDLPNLTGYQRRAFYWALENDPS | |
| | QSSELLSEAKKLNDSQAPK (45) | |
| FG1_C2 | AEAKYNKEMPDANCQIAFLPNLTQYQVPAFIYALCNDPSQ | |
| | SSELLSEAKKLNDSQAPK (46) | |
| FG1_C3 | AEAKYNKEGEDATTQIGSLPNLTQAQKHAFAVALGNDPS | |
| | QSSELLSEAKKLNDSQAPK (47) | |
| FG1_C4 | AEAKYSKEGFYADWVIPVLPNLTRKQRVAFHDALHNDPS | |
| | QSSELLSEAKKLNDSQAPK (48) | |
| FG2-C3 | AEAKYAKEWLDAIDVIGYLPNLTDFORGAFYDALNDDPS | |
| | QSSELLSEAKKLNDSQAPK (49) | |
| FG3_C2 | AEAKYYKEGYNAIVEIRCLPNLTDCQVAAFIDALDDDPSQ | |
| | SSELLSEAKKLNDSQAPK (50) | |
| FG3-C3 | AEAKYAKELDAAYVVIYFLPNLTHCQMVAFLHALSDDPS | |
| | QSSELLSEAKKLNDSQAPK (51) | |
| FG4-C1 | AEAKYSKEVYSAYDVIFALPNLTQYQVLAFFDALCDDPSQ | |
| | SSELLSEAKKLNDSQAPK (52) | |
| FG4-C2 | AEAKYAKERLTAVCSIVALPNLTEGQMVAFDDALHDDPS | |
| | QSSELLSEAKKLNDSQAPK (53) | |
| FG4-C3 | AEAKYAKEGFNAVNVIWPLPNLTADQVCAFICALADDPS | |
| | QSSELLSEAKKLNDSQAPK (54) | |
| FG4-C4 | AEAKYAKEGCTAFLEIAALPNLTGYQRDAFIEALFDDPSQ | |
| | SSELLSEAKKLNDSQAPK (55) | |
| FG2-CA | AEAKYTKEGSDAFDVIVLLPNLTRDQRDAFLYALLDDPSQ | |
| | SSELLSEAKKLNDSQAPK (56) | |

PDGF Affibodies

| | | |
|---|---|---|
| BR6 | AEAKYYKEWDSASDSIGFLPNLTRAQMVAFFAALENDPS | |
| | QSSELLSEAKKLNDSQAPK (57) | |
| 0010 (High) | AEAKYAHELWEADWEITNLPNLSPDQLMAFYMALWDDP | 1.5 |
| | SQSSELLSEAKKLNDSQAPK (58) | |
| 0057 (High) | AEAKYAFELWEAQHEIQQLPNLRPDQIAAFAMALYDDPS | 5.5 |
| | QSSELLSEAKKLNDSQAPK (59) | |
| BM_6 | AEAKYAKELDDASVEIWDLPNLTPCQKVAFFVALYDDPS | 855 |
| (Medium) | QSSELLSEAKKLNDSQAPK (60) | |
| 0032 (High) | AEEKYMMEAHWALMEILNLPNLHPCQQDAFWLALWDD | K_{D}1 = 244 |
| | PSQSSELLSEAKKLNDSQAPK (74) | nM; |
| | | K_{D}2 = 0.6 |
| | | nM |

IL-4 Affibodies

| | | |
|---|---|---|
| G3H-C3 | AEAKYNKELDAADADVEIWLLPNLTLDQLLAFIAALFNDP | 4 |
| | SQSSELLSEAKKLNDSQAPK (61) | |
| G3H-C7 | AEAKYTKELSDANAEIWSLPNLTVDQLVAFIFALWDDPSQ | 92000 |
| | SSELLSEAKKLNDSQAPK (62) | |
| G3H-C10 | AEAKYSKEQSNAYASITDLPNLTRLQKLAFWVALENDPSQ | |
| | SSELLSEAKKLNDSQAPK (63) | |
| AD_189 | AERKYHWELLVAFMEIQSLPNLTKDQITQFMAALEDDPS | |
| | QSSELLSEAKKLNDSQAPK (64) | |

The exemplary affibody sequences provided in Table 1 and variants thereof can be further linked to a hexahistidine tag (SEQ ID NO: 86), e.g., at its C-terminal or N-terminus. Exemplary affibody sequences including the tag at the C-terminus are provided below:

High Affinity BMP-2 Affibody
(AEAKYYKEVSSAATQIRYLPNLTAFQKAAFYAALLDD
PSQSSELLSEAKKLNDSQAPKHHHHHHC;
SEQ ID NO: 65)
Moderate Affinity BMP-2 Affibody
(AEAKYAKEQFNAYVVIFYLPNLTASQKAAFVDALSND
PSQSSELLSEAKKLNDSQAPKHHHHHHC;
SEQ ID NO: 66)
Low Affinity BMP-2 Affibody
(AEAKYYKEGDNAYNVIYGLPNLTRPQRLAFIVALFND
PSQSSELLSEAKKLNDSQAPKHHHHHHC;
SEQ ID NO: 67)
High Affinity GM-CSF Affibody
(AEAKYTKELFNAVGEITALPNLTRYHLYAFYYALLND
PSQSSELLSEAKKLNDSQAPKHHHHHHC;
SEQ ID NO: 68)

TABLE 2
Exemplary dual-affinity fusion protein
sequences(SEQ ID NO: in parenthesis).

| Localization Domain | Linkers | Affibody Sequences | Optional His-tag |
|---|---|---|---|
| Collagen binding: | Flexible: | Any affibody | HHHHHH(86) |
| TKKTLRT (75) | GGGGS(80) | sequence and variants | |
| | GGGGSGGGGSGGGGS | thereof disclosed | |
| | (81) | herein, e.g., SEQ ID | |
| Collagen binding: | Rigid: | NOs: 1-68 and 71-74 | |
| TKKLTLRT (76) | AEAAAKEAAAKA (82) | with or without the | |
| | LAEAAAKAAA (83) | initial A. | |
| HA binding: | Hybrid: | | |
| EEEEEEEDGEA (77) | GGGAAAA (84) | | |
| | EAAAKAGGGGS (85) | | |
| HA binding: | | | |
| GKNFQS (78) | | | |
| HA binding: | | | |
| SVSVSVK (79) | | | |
| HA binding: | | | |
| KNFQSRSH (127) | | | |

Exemplary localization domains include those set forth in SEQ ID NOs: 75-79 and 127. In some examples, the localization domain has 1, 2, 3, 4, 5 or 6 conservative amino acid substitutions based on any of SEQ ID NOs: 75-79 and 127. In some examples, the collagen binding domain, SEQ ID NO: 75 or 76, further includes an optional G (Gly) at the beginning of the sequence. In some examples, the HA binding domain, SEQ ID NO: 79, further includes an optional G (Gly) at the beginning of the sequence. In some examples, the HA binding domain, SEQ ID NO: 78, further includes an optional RSH (Arg-Ser-His) at the end of the sequence.

Exemplary linker sequences include those set forth in SEQ ID NOs: 80-85 and 120-125. In some examples, the linker has 1, 2, 3, 4, 5 or 6 conservative amino acid substitutions based on any of SEQ ID NOs: 80-85 and 120-125. In some examples, the linker sequence includes any combination of SEQ ID NOs: 80-85 and 120-125, such as SEQ ID NO: 80 plus SEQ ID NO: 82, or SEQ ID NO: 84 plus SEQ ID NO: 82.

Any of the localization domain sequences (including variants thereof) disclosed herein can be linked to any of the affibody sequences (including variants thereof) disclosed herein, through any of the linker sequences (including variants thereof) disclosed herein, to form the dual-affinity fusion proteins. In some aspects, the localization domain is at the N-terminus of the fusion protein, and the affibody is at the C-terminus of the fusion protein. In some aspects, the localization domain is at the C-terminus of the fusion protein, and the affibody is at the N-terminus of the fusion protein. The fusion proteins can optionally include a His-tag (e.g., SEQ ID NO: 86) at one or both of its terminal ends (e.g., N- or C-terminal). In some examples, the fusion protein exhibits a higher affinity (e.g., a K_{D} of 10⁻¹⁰⁰⁰ nm) towards the structural bone component than towards the therapeutic protein (e.g., a K_{D} of 1000-100,000 nM). In some examples, the fusion protein exhibits approximately the same affinity for the structural bone component and for the therapeutic protein (e.g., a K_{D} of 50-500 nM). In some examples, the fusion protein exhibits a lower affinity towards the structural bone component (e.g., a K_{D} of 200-500 nM) than towards the therapeutic protein (e.g., a K_{D} of 10⁻¹⁰⁰ nM); for example, the K_{D} for therapeutic protein binding is less than an order of magnitude lower than that for structural bone component binding. Exemplary fusion proteins include those set forth in SEQ ID NOs: 87-112 and 126. Exemplary coding sequences for these proteins include those set forth in SEQ ID NOs: 113-119.

Exemplary collagen binding and BMP-2 binding fusion proteins and coding DNA:

(SEQ ID NO: 87)
GTKKTLRTGGGGSEAKYYKEVSSAATQIRYLPNLTAFQKAAFYAALLDD
PSQSSELLSEAKKLNDSQAPK
(SEQ ID NO: 113)
GGCACTAAGAAGACCCTGCGCACGGGCGGAGGGGGGTCGGCAGAAGCGA
AATACGCGAAAGAACAGTTCAACGCCTACGTCGTAATCTTCTACTTACC
AAACCTGACAGCATCTCAGAAAGCTGCGTTTGTTGATGCATTAAGTAAT
GATCCATCCCAAAGCTCCGAGTTGCTGAGTGAGGCCAAGAAACTTAACG
ATAGCCAGGCTCCGAAG
(SEQ ID NO: 88)
GTKKLTLRTAEAAAKEAAAKAEAKYYKEVSSAATQIRYLPNLTAFQKAA
FYAALLDDPSQSSELLSEAKKLNDSQAPK
(SEQ ID NO: 114)
GGAACCAAGAAGCTGACATTACGCACCGCCGAGGCGGCTGCGAAGGAAG
CGGCTGCCAAGGCCGAGGCAAAATATTACAAAGAAGTTAGCTCCGCCGC
AACGCAAATTCGCTATTTACCCAATTTAACAGCGTTCCAGAAAGCCGCG
TTCTACGCTGCCTTGCTTGATGATCCGAGCCAAAGTTCGGAGCTGTTAT
CAGAAGCAAAGAAACTGAACGATAGTCAAGCGCCAAAA
(SEQ ID NO: 89)
GTKKLTLRTGGGGSEAKYYKEVSSAATQIRYLPNLTAFQKAAFYAALLD
DPSQSSELLSEAKKLNDSQAPK
(SEQ ID NO: 90)
GTKKLTLRTGGGGSEAKYAKEQFNAYVVIFYLPNLTASQKAAFVDALSN
DPSQSSELLSEAKKLNDSQAPK
(SEQ ID NO: 91)
GTKKLTLRTGGGGSEAKYYKEGDNAYNVIYGLPNLTRPQRLAFIVALEN
DPSQSSELLSEAKKLNDSQAPK

Exemplary collagen binding and GM-CSF binding fusion proteins:

(SEQ ID NO: 92)
GTKKLTLRTGGGGSEAKYTKELFNAVGEITALPNLTRYHLYAFYYALLN
DPSQSSELLSEAKKLNDSQAPK
(SEQ ID NO: 93)
GTKKLTLRTGGGGSEAKYNKEWFAADLSIGFLPNLTLDQLYAFVFALYD
DPSQSSELLSEAKKLNDSQAPK
(SEQ ID NO: 94)
GTKKLTLRTGGGGSEAKYAKEGLNAYLSIRWLPNLTGDQMYAFISALLD
DPSQSSELLSEAKKLNDSQAPK

Exemplary collagen binding and IL-4 binding fusion proteins:

(SEQ ID NO: 95)
GTKKLTLRTGGGGSEAKYNKELDAADADVEIWLLPNLTLDQLLAFIAAL
ENDPSQSSELLSEAKKLNDSQAPK
(SEQ ID NO: 96)
GTKKLTLRTGGGGSEAKYTKELSDANAEIWSLPNLTVDQLVAFIFALWD
DPSQSSELLSEAKKLNDSQAPK

Exemplary collagen binding and PDGF binding fusion proteins:

(SEQ ID NO: 97)
GTKKLTLRTGGGGSEEKYMMEAHWALMEILNLPNLHPCQQDAFWLALWD
DPSQSSELLSEAKKLNDSQAPK
(SEQ ID NO: 98)
GTKKLTLRTGGGGSEAKYYKEWDSASDSIGFLPNLTRAQMVAFFAALFN
DPSQSSELLSEAKKLNDSQAPK
(SEQ ID NO: 99)
GTKKLTLRTGGGGSEAKYAHELWEADWEITNLPNLSPDQLMAFYMALWD
DPSQSSELLSEAKKLNDSQAPK
(SEQ ID NO: 100)
GTKKLTLRTGGGGSEAKYAFELWEAQHEIQQLPNLRPDQIAAFAMALYD
DPSQSSELLSEAKKLNDSQAPK
(SEQ ID NO: 101)
GTKKLTLRTGGGGSEAKYAKELDDASVEIWDLPNLTPCQKVAFFVALYD
DPSQSSELLSEAKKLNDSQAPK

Exemplary collagen binding and VEGF binding fusion proteins:

(SEQ ID NO: 102)
GTKKLTLRTGGGGSEAKYNKEWYDAVFVIGSLPNLTEDQKAAFSDALVD
DPSQSSELLSEAKKLNDSQAPK
(SEQ ID NO: 103)
GTKKLTLRTGGGGSEAKYNKEWYDAVFVIGSLPNLTEDQKDAFSAALVD
DPSQSSELLSEAKKLNDSQAPK
(SEQ ID NO: 104)
GTKKLTLRTGGGGSEAKYNKEWYDAVFVIGSLPNLTEDQKDAFSDALVA
DPSQSSELLSEAKKLNDSQAPK
(SEQ ID NO: 105)
GTKKLTLRTGGGGSEAKYYKEGATAYRVIEYLPNLTGAQKAAFIDALYN
DPSQSSELLSEAKKLNDSQAPK
(SEQ ID NO: 106)
GTKKLTLRTGGGGSEAKYTKEGFDAYDVIDNLPNLTLDQRNAFVYALFN
DPSQSSELLSEAKKLNDSQAPK
(SEQ ID NO: 107)
GTKKLTLRTGGGGSEAKYNKEWYDAVFVIGSLPNLTEDQKDAFSDALVD
DPSQSSELLSEAKKLNDSQAPK

Exemplary HA binding and BMP-2 binding fusion proteins:

(SEQ ID NO: 108)
EEEEEEEDGEAAEAAAKEAAAKAEAKYYKEVSSAATQIRYLPNLTAFQK
AAFYAALLDDPSQSSELLSEAKKLNDSQAPK
(SEQ ID NO: 115)
GAAGAGGAAGAGGAGGAGGAGGATGGTGAAGCCGCTGAGGCCGCTGCAA
AAGAAGCTGCAGCTAAGGCCGAAGCTAAGTATTACAAGGAGGTAAGTTC
TGCTGCGACACAGATCCGTTATCTTCCGAATTTAACTGCCTTCCAAAAG
GCGGCGTTCTACGCGGCTCTTTTGGATGATCCATCACAATCGTCCGAAT
TGCTTAGCGAAGCGAAAAAATTGAATGACTCCCAAGCGCCCAAG
(SEQ ID NO: 109)
GKNFQSRSHAEAAAKEAAAKAEAKYYKEVSSAATQIRYLPNLTAFQKAA
FYAALLDDPSQSSELLSEAKKLNDSQAPK
(SEQ ID NO: 116)
GGAAAGAACTTCCAAAGTCGTAGTCATGCTGAAGCCGCAGCTAAGGAAG
CTGCGGCAAAGGCCGAAGCAAAATACTACAAAGAAGTGTCGAGTGCCGC
CACCCAAATCCGCTATCTTCCGAACTTGACAGCATTTCAGAAGGCCGCA
TTCTACGCCGCCTTGCTGGACGATCCCTCTCAGTCATCAGAGCTGTTAA
GCGAGGCAAAAAAACTGAATGATTCACAGGCTCCGAAG
(SEQ ID NO: 110)
GKNFQSRSHGGGAAAAEAKYYKEVSSAATQIRYLPNLTAFQKAAFYAAL
LDDPSQSSELLSEAKKLNDSQAPK
(SEQ ID NO: 117)
GGGAAAAACTTTCAATCTCGTAGCCACGGAGGGGGTGCGGCAGCAGCGG
AGGCGAAATATTACAAAGAAGTTAGTTCAGCGGCAACTCAAATTCGCTA
CTTACCGAATTTAACGGCGTTTCAAAAAGCCGCGTTCTATGCCGCCTTA
CTGGACGACCCTAGTCAATCTTCTGAGCTTTTGAGTGAAGCCAAAAAAT
TGAACGACTCACAAGCACCTAAG
(SEQ ID NO: 111)
GSVSVSVKAEAAAKEAAAKAEAKYYKEVSSAATQIRYLPNLTAFQKAAF
YAALLDDPSQSSELLSEAKKLNDSQAPK
(SEQ ID NO: 118)
GGAAGTGTAAGTGTCTCGGTCAAAGCAGAAGCAGCGGCTAAAGAGGCAG
CCGCCAAAGCCGAGGCTAAATACTATAAGGAAGTATCAAGCGCAGCAAC
TCAAATCCGCTACCTGCCTAACCTGACTGCGTTTCAAAAGGCCGCTTTC
TATGCAGCGTTGTTGGATGACCCTAGCCAGTCGTCCGAGCTTTTAAGTG
AGGCAAAAAAATTAAATGATTCCCAAGCGCCGAAG
(SEQ ID NO: 112)
GSVSVSVKGGGAAAAEAKYYKEVSSAATQIRYLPNLTAFQKAAFYAAL
LDDPSQSSELLSEAKKLNDSQAPK
(SEQ ID NO: 119)
GGGAGTGTCTCTGTTAGTGTCAAGGGCGGTGGGGCGGCGGCTGCGGAAG
CCAAGTATTACAAGGAAGTCTCCAGCGCAGCAACTCAAATCCGCTACTT
ACCCAATTTAACAGCATTTCAAAAAGCCGCCTTTTACGCGGCCCTGCTT
GACGACCCTTCTCAAAGTAGTGAACTTTTGAGTGAGGCTAAGAAACTGA
ACGACAGCCAGGCACCAAAG
(SEQ ID NO: 126)
EEEEEEEDGEAGGGAAAAEAKYYKEVSSAATQIRYLPNLTAFQKAAFYA
ALLDDPSQSSELLSEAKKLNDSQAPK

In one example, provided are one or more fusion proteins each including the BMP-2 affibody domain of any of SEQ ID NOs: 1-11 or 65-67, with or without the initial A, which in some examples are present in a composition. Such a composition can further include BMP-2, and a medical material for treating wound, or a bone or cartilage injury or disease, and can be used to control release of BMP-2 from the medical material, for example in the treatment of a bone or cartilage injury (for example by applying the composition to an injury site on bone or cartilage).

In one example, provided are one or more fusion proteins each including the GM-CSF affibody domain of any of SEQ ID NOs: 12-19 or 68, with or without the initial A, which in some examples are present in a composition. Such a composition can further include GM-CSF, and a medical material for treating wound, or a bone or cartilage injury or disease, and can be used to control release of GM-CSF from the medical material, for example in the treatment of a wound or bone or cartilage injury (for example by applying the composition to a wound or injury site).

In one example, provided are one or more fusion proteins each including the VEGF affibody domain of any of SEQ ID NOs: 20-41 and 71-73, with or without the initial A, which in some examples are present in a composition. Such a composition can further include VEGF, and a medical material for treating wound, or a bone or cartilage injury or disease, and can be used to control release of VEGF from the medical material, for example to stimulate angiogenesis, for example in the treatment of a wound or bone, muscle or cartilage injury (for example by applying the composition to a wound or injury site).

In one example, provided are one or more of fusion proteins each including the FGF-2 affibody domain of any of SEQ ID NOs: 42-56, which in some examples are present in a composition. Such a composition can further include FGF-2, and a medical material for treating wound, or a bone, muscle or cartilage injury or disease, and can be used to control release of FGF-2 from the medical material, for example in the treatment of a wound or bone or cartilage injury (for example by applying the composition to a wound or injury site).

In one example, provided are one or more of fusion proteins each including the PDGF affibody domain of any of SEQ ID NOs: 57-60 and 74, which in some examples are present in a composition. Such a composition can further include PDGF, and a medical material for treating wound, or a bone or cartilage injury or disease, and can be used to control release of PDGF from the medical material, for example in the treatment of a wound or bone or cartilage injury (for example by applying the composition to a wound or injury site).

In one example, provided are one or more fusion proteins each including the IL-4 affibody domain of any of SEQ ID NOs: 61-64, which in some examples are present in a composition. Such a composition can further include IL-4, and a medical material for treating wound, or a bone, muscle or cartilage injury or disease, and can be used to control release of IL-4 from the medical material, for example in the treatment of a wound by manipulating the immune response to injury (for example by applying the composition to a wound or injury site).

### Methods of Treatment

The present disclosure provides methods of using the disclosed dual-affinity fusion proteins and compositions thereof to treat a disease, by administering the composition to provide for an effective amount of the one or more therapeutic proteins released over a period of time in a subject in need thereof. In some examples, a medical material including two or more (such as 2, 3, 4, or 5 different) fusion proteins specific for two or more therapeutic proteins are used in a treatment. Such administration can be localized. In some examples, the composition is administered (such as placed or injected) directly to an injury, void, or defect site, for example as part of a surgical procedure. In some examples, multiple administrations are performed. The subject treated can be a mammal, such as a human or veterinary subject. Exemplary diseases/injuries that can be treated are provided in Table 2, with the appropriate fusion proteins/therapeutic proteins listed.

TABLE 3
Exemplary treatments

| | Exemplary Therapeutic |
|---|---|
| | Proteins and |
| | Corresponding |
| Disease/Injury | Fusion Proteins |
| Bone or cartilage (e.g., | BMP-2 |
| fracture, cancer, | GM-CSF |
| osteoporosis, | IL-4 |
| osteoarthritis). | BMP-2 + GM-CSF |
| | BMP-2 + IL-4 |
| | BMP-2 + GM-CSF + IL-4 |
| | BMP-2 + PDGF |
| | BMP-2 + VEGF |
| | BMP-2 + PDGF + VEGF + FGF-2 |
| Wound, vascular | VEGF |
| injury, muscle | FGF-2 |
| injury | PDGF |
| | GM-CSF |
| | IL-4 |
| | VEGF + FGF-2 |
| | VEGF + PDGF |
| | VEGF + FGF-2 + PDGF |
| | VEGF + FGF-2 + PDGF + GM-CSF |
| | VEGF + FGF-2 + PDGF + IL-4 |
| | VEGF + FGF-2 + PDGF + GM-CSF + IL-4 |

In one example, the subject has a bone or cartilage injury, and the method includes administering the medical material composition to the site of injury, and the composition includes one or more fusion proteins specific for BMP-2, one or more fusion proteins specific for IL-4, and/or one or more fusion proteins specific for GM-CSF, and in some examples the composition further includes BMP-2, IL-4, and/or GM-CSF. Exemplary bone injuries include fractures (such as those caused by trauma), for example in the spinal column, vertebrae (such as the lumbar vertebra), femur, tibia, fibula, thoracic cage, rib, clavicle, humerus, radius, ulna, tarsal bone, ilium, cranium, carpal bone, or a bone of the face (such as a mandible, nasal, zygomatic, lacrimal, maxilla, or sphenoid bone). In one example, the bone injury results from loss of bone, for example due to surgery, cancer, osteoporosis, osteoarthritis or other disease or injury. In one example, a subject is administered a medical material composition that includes one or more (such as at least 2, at least 3, or 1, 2, 3, 4, 5, 6, 7, 8, 9, 10, or 11) fusion proteins each including a BMP-2 affibody domain of any of SEQ ID NOs: 1-11, with or without the initial A, or a BMP-2 affibody domain having at least 90% or at least 95% sequence identity to any of SEQ ID NOs: 1-11, with or without the initial A), and in some examples the composition further includes BMP-2. In some examples, the oner or more fusion proteins comprise or consist of any one of SEQ ID NOs: 87-91, 108-112, and 126 or have at least 90% or at least 95% sequence identity to any one of SEQ ID NOs: 87-91, 108-112, and 126. In one example, a subject is administered a medical material composition that includes one or more (such as at least 2, at least 3, or 1, 2, 3, or 4) fusion proteins each including an IL-4 affibody domain of any of SEQ ID NOs: 61-64, with or without the initial A, or an IL-4 affibody domain having at least 90% or at least 95% sequence identity to any of SEQ ID NOs: 61-64, with or without the initial A, and in some examples the composition further includes IL-4. In one example, a subject is administered a medical material composition that includes one or more (such as at least 2, at least 3, or 1, 2, 3, 4, 5, 6, 7, or 8) fusion proteins each including a GM-CSF affibody domain of any of SEQ ID NOs: 12-19, with or without the initial A, or a GM-CSF affibody domain having at least 90% or at least 95% sequence identity to any of SEQ ID NOs: 12-19, with or without the initial A, and in some examples the composition further includes GM-CSF. In some examples, the medical material composition includes combinations of these affibodies.

In one example, the subject has an injury or disease that would benefit from increase angiogenesis, and the method includes administering a medical material composition to the site of injury, and the composition includes one or more fusion proteins specific for VEGF, one or more fusion proteins specific for PDGF, one or more fusion proteins specific for GM-CSF, and/or one or more fusion proteins specific for FGF-2, and in some examples the composition further includes VEGF, PDGF, GM-CSF, and/or FGF-2. Angiogenesis, the process through which new blood vessels form, is a component of musculoskeletal healing, as it enables the transport of biomolecules to an injury site. Angiogenesis is mediated by a signaling cascade of key proteins; however, the temporal presentation of these proteins may be disrupted by factors such as age, severe injury severity, and chronic disease. Supplementation of angiogenic proteins, including VEGF, FGF-2, GM-CSF, and PDGF, using the fusion proteins provided herein, provides a method to stimulate angiogenesis. In one example, increased angiogenesis is used to treat a wound, such as one on the skin. Exemplary wound that can be treated include penetrating wounds, thermal burn, chemical burn, electric burn, surgical wound, puncture wounds, lacerations, abrasions, skin tears and diabetic ulcers. In some examples, increased angiogenesis is used to facilitate healing of wound, or growth of bone or cartilage.

In one example, a subject with a wound, or a bone or cartilage injury or disease is administered a medical material composition that includes one or more (such as at least 2, at least 3, or 1, 2, 3, 4, 5, 6, 7, or 8) fusion proteins each including a GM-CSF affibody domain of any of SEQ ID NOs: 12-19, with or without the initial A, or an affibody domain having at least 90% or at least 95% sequence identity to any of SEQ ID NOs: 12-19, with or without the initial A, and in some examples the composition further includes GM-CSF. In one example, a subject with a wound, or a bone or cartilage injury or disease is administered a medical material composition that includes one or more (such as at least 2, at least 3, or 1, 2, 3, 4, 5, 6, 7, 8, 9, 10, 11, 12, 13, 14, 15, 16, 17, 18, 19, 20, 21 or 22) fusion proteins each including a VEGF affibody domain of any of SEQ ID NOs: 20-41 and 71-73, with or without the initial A, or an affibody domain having at least 90% or at least 95% sequence identity to any of SEQ ID NOs: 20-41 and 71-73, with or without the initial A, and in some examples the composition further includes VEGF. In one example, a subject with a wound, or a bone or cartilage injury or disease is administered a medical material composition that includes one or more (such as at least 2, at least 3, or 1, 2, 3, 4, 5, 6, 7, 8, 9, 10, 11, 12, 13, 14, or 15) fusion proteins each including a FGF-2 affibody domain of any of SEQ ID NOs: 42-56, with or without the initial A, or an affibody domain having at least 90% or at least 95% sequence identity to any of SEQ ID NOs: 42-56, with or without the initial A, and in some examples the composition further includes FGF-2. In one example, a subject with a wound, or a bone or cartilage injury or disease is administered a medical material composition that includes one or more (such as at least 2, at least 3, or 1, 2, 3, or 4) fusion proteins each including a PDGF affibody domain of any of SEQ ID NOs: 57-60 and 74, with or without the initial A, or an affibody domain having at least 90% or at least 95% sequence identity to any of SEQ ID NOs: 57-60 and 74, with or without the initial A, and in some examples the composition further includes PDGF. In some examples, the medical material composition includes combinations of these fusion proteins and corresponding therapeutic proteins.

### Example 1: Materials and Methods

This example provides the materials and methods used to generate the results described in Examples 2-16.

### Protein Modifications

Recombinant human BMP-2 (Medtronic, R&D Systems) was biotinylated using EZ-Link™ Sulfo-NHS-Biotin (Thermo Fisher) per the manufacturer's protocols. Briefly, a 10 mM solution of sulfo-NHS-biotin in water was prepared, and 20 molar excess of sulfo-NHS-biotin was added to a 0.5 mg mL⁻¹ solution of BMP-2 (Medtronic) in phosphate buffered saline (Fisher Scientific; PBS). The reaction was carried out for 2 hours at 4° C., and the biotinylated product (bBMP-2) was eluted into PBS using 7 kDa Zeba Spin Desalting Column (Thermo Fisher)). Biotinylation was confirmed using a Pierce™ Biotin Quantitation Kit (Thermo Fisher)).

### Yeast Growth and Induction

The naïve affibody-expressing yeast surface display library used was donated by Dr. Benjamin Hackel. This EBY100 strain of S. cerevisiae contains the pCT surface display vector for galactose-inducible surface protein expression of roughly 4×10⁸ unique affibody sequences.⁴³ Yeast were grown in selective growth media (16.8 g sodium citrate dihydrate, 3.9 g citric acid, 20.0 g dextrose, 6.7 g yeast nitrogen base, 5.0 g casamino acids, 1 mg ciprofloxacin and 100 mg ampicillin in 1 L reverse osmosis (RO) water) in an Innova44 shaking incubator (Innova) at 37° C. for 20 hours to a concentration between 5-10×10⁷ cells mL⁻¹, after which 10× library diversity was transferred into selective induction media (10.2 g sodium phosphate dibasic heptahydrate, 8.6 g sodium phosphate monobasic monohydrate, 19.0 g galactose, 1.0 g dextrose, 6.7 g yeast nitrogen base, 5.0 g casamino acids, 1 mg ciprofloxacin and 100 mg ampicillin in 1 L RO water) to induce affibody expression in a shaking incubator at 37° C. for 20 hours.

Surface protein expression was confirmed by flow cytometry. 1×10⁶ cells were aliquoted into tubes labeled cells only, secondary only, and c-myc+secondary. Each tube was washed and resuspended in 50 μL of PBS+0.1% BSA (PBSA). 1.25 μL of anti-c-myc mouse monoclonal antibody (αCMYC, 9E10; BioLegend) were added to the c-myc+secondary tube. The tubes were rotated at 4° C. for 30 minutes. All tubes were washed again and resuspended in 50 μL of PBSA. 0.625 μL of goat anti-mouse IgG-AlexaFluor™ 488 secondary antibody (Thermo Fisher; AF488) were added to the secondary only and c-myc+secondary tubes. All tubes were rotated for 30 minutes in the dark at 4° C. All tubes were washed twice and resuspended in 200 μL PBSA. Flow cytometry was performed using an Accuri™ C6 Plus Flow Cytometer with 96-well plate autosampler (Becton Dickinson).

### Magnetic Activated Cell Sorting

Magnetic-activated cell sorting (MACS) was performed to enrich for BMP-2-binding affibodies within the yeast surface display library. One round of MACS consisted of two negative bead sorts and one positive bead sort. Negative bead sorts were performed using carboxylic acid magnetic beads (COOH beads) conjugated with either tris or BSA, which removed non-specific binders.^{39,40,50,51} The positive bead sorts consisted of COOH beads conjugated with BMP-2 to enrich for yeast displaying BMP-2-specific affibodies. To prepare the beads, 2 μL of COOH beads (Invitrogen™ Dynabeads™ M-270 Carboxylic Acid) were rotated with 100 μL of cold 0.05 M NaOH for 10 minutes and then exposed to a magnetic field for 2 minutes so that a magnetic bead pellet formed at the wall of the tube. The NaOH was carefully removed to avoid disturbing the pellet, and the beads were then resuspended in 100 μL of cold water and rotated for 10 minutes. The beads were then resuspended and rotated in 100 μL of 50 mg mL⁻¹ solution of 1-Ethyl-3-(3-dimethylaminopropyl)carbodiimide (EDC) in water for 30 minutes. The EDC solution was removed, and the beads quickly rinsed with cold water and resuspended in 100 μL of 0.1 M MES buffer pH 5 followed by either 500 μL of PBSA, 500 μL of 0.05 M tris pH 7.4, or 33 pmol of carrier-free BMP-2 (R&D Biosystems) in water and rotated for 30 minutes. The reaction was terminated using the 0.05 M tris pH 7.4, and the beads were washed and resuspended in a solution of PBSA and stored on ice until needed.

10× library diversity was washed in PBSA to remove the induction media and resuspended in BSA-conjugated COOH bead solution. The yeast and beads were rotated at 4° C. for 2 hours and then exposed to a magnetic field. The unbound solution was gently removed and transferred to a tube containing tris-conjugated magnetic beads. The rotation and exposure were repeated as above, and the unbound solution was transferred to a tube with the BMP-2-conjugated magnetic beads and rotated once again for 2 hours. After exposure to the magnetic field, the unbound solution was removed, and the magnetic beads were resuspended in PBSA. 10 μL of 100× and 2000× diluted BSA, tris, and BMP-2-conjugated beads were plated on selective growth plates (16.8 g sodium citrate dihydrate, 3.9 g citric acid, 16 g bacto agar, 20 g dextrose, 6.7 g yeast nitrogen base, 5 g casamino acids, RO water, autoclaved and poured into petri dishes). Plates were incubated at 30° C. for 36 h, and colonies were counted to determine the ratio of positive-to-negative binders and new library diversity. The updated library diversity was estimated by the formula below, and the new diversity was used to determine the number of yeasts used for subsequent sorts.

$$library diversity = ( \frac{CFU}{plate} ) * ( dilution factor ) * ( \frac{total volume of undiluted positive sort}{10 μ L} )$$

### Fluorescence-Activated Cell Sorting

Fluorescence-activated cell sorting (FACS) was performed on the enriched yeast library after MACS to separate yeast into populations corresponding to approximately different affinity ranges for BMP-2 binding. 40×10⁶ induced yeast cells were aliquoted into tubes labeled cells only, secondary only, c-myc, and c-myc+bBMP-2, and washed in PBSA. The cells only, secondary only and c-myc tubes were resuspended in 50 μL PBSA. The c-myc+bBMP-2 tube was resuspended in 50 μL of 1 μM bBMP-2 in PBSA. 1.25 μL of αCMYC were also added to the c-myc and c-myc+bBMP-2 tubes. All tubes were rotated at 4° C. for 1 h and then washed with PBSA. Except for the cells-only control, all tubes were incubated with 50 μL of secondary fluorescent solution (10.4 μL of 333 nM goat anti-mouse IgG AlexaFluor™ 647, 3.25 μL of AlexaFluor™ 488 streptavidin conjugate, 187 μL PBSA). The tubes were all rotated at 4° C. for 30 minutes and washed 2 times in 500 μL of PBSA. The yeast was suspended in 1000 μL PBSA and sorted by a SH800 Cell Sorter (Sony Biotechnology). At least 10,000 cells were obtained from each gate. Following FACS, yeast from each collected gate were grown in selective growth media at 30° C. to an approximate concentration of 10⁷ cells mL⁻¹, plated onto selective growth plates, and incubated for 24-36 hours in 30° C.

### Gene Sequencing of Monoclonal Affibody Yeast

Individual colonies from FACS-sorted yeast plates were selected and expanded in yeast growth media to a cell density of 10⁷ cells mL⁻¹. The yeast plasmids were isolated using Easy Yeast Plasmid Isolation Kit (Clontech) per the manufacturer's instructions. The affibody sequences from the plasmids were amplified by PCR in an Applied Biosystems Thermocycler (Fisher Scientific) using HiFi PCR Premix (CloneAmp) and forward primer (5′-CCCTCAACAACTAGCAAAGG-3′; SEQ ID NO: 69) and reverse primer (3′-ATGTGTAAAGTTGGTAACGGAACG-5′; SEQ ID NO: 70) for 35 cycles and purified using a DNA Clean and Concentrator Kit (ZymoGen). The purified products were submitted for Sanger Sequencing to GeneWiz® (Azenta Life Sciences).

### Monoclonal Affibody Yeast Characterization

The binding affinity of each unique affibody for BMP-2 was characterized using flow cytometry. Samples were prepared similarly to the FACS procedure with the following differences: 1×10⁶ induced cells were used in each tube instead of 40×10⁶ cells, c-myc+bBMP-2 tubes were prepared with bBMP-2 concentrations ranging from 0.5-1000 nM, and each tube was resuspended in 200 μL of PBSA and transferred to a 96-well plate. Flow cytometry was performed on bBMP-2-containing samples in triplicate. Cells were analyzed using Accuri™ C6 Plus Flow Cytometer with 96-well plate autosampler (Becton Dickinson).

To quantify the equilibrium dissociation constant (K_{D}) of affibody-BMP-2 binding, the ratio of AF647+/AF488+ cells to AF647+ cells was calculated at each bBMP-2 concentration and plotted against protein concentration. Nonlinear regression was performed, in which the equilibrium dissociation constant was the inflection point of the curve.

Specificity of the affibodies to BMP-2 was confirmed using flow cytometry in a similar manner, except that 1 μM solutions of bVEGF (R&D Biosystems), bIL-4 (Acro Biosystems), and bGM-CSF (Acro Biosystems) were used.

Transformation of BMP-2-Specific Affibody Vectors into E. coli

pET28b+ expression vectors containing sequences for each of the unique BMP-2-specific affibodies modified with a methionine at the N-terminus and a 6-His-tag and cysteine at the C-terminus were prepared by GenScript. The pET28b+ vector confers kanamycin resistance and uses an isopropyl ß-D-1-thiogalactopyranoside (IPTG)-inducible T7 promoter for protein expression. Vectors were transformed into BL21 chemically competent E. coli (New England BioLabs) per the manufacturer's protocols. 100 μL of transformed E. coli were plated on kanamycin selective growth plates (10 g yeast extract, 20 g bacto peptone, 20 g dextrose, 16 g bacto agar, 50 mg kanamycin sulfate, and 1 L RO water) and incubated at 37° C. for 24 h. Colonies were selected and expanded in 20 mL Luria-Bertani (LB) broth (Thermo Fisher) supplemented in 20 μL of 50 mg mL⁻¹ of kanamycin sulfate in water until an optical density at 600 nm (OD600) of 0.8 was reached. 4 mL of the culture were lysed and used to obtain plasmid DNA for sequence confirmation (Plasmid Miniprep Kit; Zymo Research), and the remaining volume was split in half, in which one half was induced with 10 μL of 0.5 M IPTG and incubated further for 4 hours at 37° C., and the other half was refrigerated at 4° C. The induced and uninduced E. coli were lysed using Bug Buster Protein Extraction Agent (Millipore Sigma) and centrifuged to separate the soluble proteins and the lysate. The soluble proteins were prepared for SDS-PAGE by diluting 18 μL of sample in 6 μL Laemmli buffer (BioRad) supplemented with 10 v/v % β-mercaptoethanol (BioRad) and heated for 5 minutes at 90° C. The sampled were loaded into a 4-20% Mini-PROTEAN® TGX™ Precast Protein Gel (BioRad), run under denaturing conditions at 200 V for 35 minutes, stained with Coomassie blue dye, and imaged on an Azure 200 Gel Imager (Azure Biosystems, Inc.).

### Collection and Purification of Soluble BMP-2-Specific Affibodies

Transformed E. coli were grown in 20 mL LB broth supplemented with kanamycin to a 0.5 mM concentration and incubated overnight at 37° C. The contents were then transferred into 1.8 L of Terrific Broth (TB) supplemented with kanamycin to 0.5 mM and 500 μL of anti-foam 204 (Thermo Scientific) and cultured at 37° C. in a LEX-10 bioreactor (Epiphyte3). When the OD600 reached approximately 1.4, 1.8 mL of 0.5 M IPTG was added to the growth vessel to obtain a final concentration of 0.5 μM, and the temperature was reduced to 18° C. for 18 hours for induction of protein expression. After 18 hours, the culture was centrifuged for 20 minutes at 4° C. at 6000 RPM, and the cell pellet was removed and transferred to two 50 mL conical tubes. Binding buffer (50 mL of 1 M tris pH 7.5, 100 mL of 5 M NaCl, 5 mL of 1 M imidazole, and 845 mL RO water) supplemented with 75 mg of tris (2-carboxyethyl) phosphine hydrochloride (TCEP; GoldBio) was added to the cell pellet to a volume of 35 mL and lysed using a probe sonicator (Fisher Scientific) for 5 minutes in an ice bath. The sonicated product was centrifuged at 13,000 rcf for 30 minutes at 4° C. The supernatant was transferred to a 50 mL conical tubes along with 3.6 mL of Nickel-NTA Agarose Beads (GoldBio; Nickel beads) and rotated at 4° C. for 45 minutes. The supernatant was then transferred to a Econo-Column® chromatograph column (Biorad), washed with 50 mL of wash buffer (50 mL of 1 M tris pH 7.5, 100 mL of 5 M NaCl, 30 mL of 1 M imidazole, and 820 mL RO water) supplemented with 125 mg of TCEP followed by with 50 mL of wash buffer without TCEP, and eluted into 10 mL of elution buffer (50 mL of 1 M tris pH 7.5, 100 mL of 5 M NaCl, 250 mL of 1 M imidazole, and 600 mL RO water). The collected solution was then buffer-exchanged into 0.5 M Tris pH 8 using a 3 kDa molecular weight cut-off (MWCO) centrifuge filter (Millipore) and frozen at −80° C. until further use. SDS-PAGE was used to determine purity of the affibody at each step. UV-vis spectroscopy (Implen NP80) at 280 nm was used to determine the final concentration of the affibodies.

Transformation of Fusion Protein Vectors into E. coli

DNA insert sequences for each of the fusion protein modified with a methionine at the N-terminus and a 6-His-tag at the C-terminus were prepared by IDT. Using a double digest restriction enzyme protocol with NcoI and XhoI cut-sites, the DNA insert was cloned into a pET28b+ vector. The pET28b+ vector confers kanamycin resistance and uses an isopropyl ß-D-1-thiogalactopyranoside (IPTG)-inducible T7 promoter for protein expression. Vectors were transformed into BL21 chemically competent E. coli (New England BioLabs) per the manufacturer's protocols. 100 μL of transformed E. coli were plated on kanamycin selective growth plates (10 g yeast extract, 20 g bacto peptone, 20 g dextrose, 16 g bacto agar, 50 mg kanamycin sulfate, and 1 L RO water) and incubated at 37° C. for 24 h. Colonies were selected and expanded in 20 mL Luria-Bertani (LB) broth (Thermo Fisher) supplemented in 20 μL of 50 mg mL⁻¹ of kanamycin sulfate in water until an optical density at 600 nm (OD600) of 0.8 was reached. 4 mL of the culture were lysed and used to obtain plasmid DNA for sequence confirmation to Plasmidsaurus (Plasmid Miniprep Kit; Zymo Research). A new overnight culture was prepared for each colony that was confirmed to contain the desired fusion protein sequence. 200 μL of culture was added to 5 mL of LB broth with 5 μL of kanamycin sulfate solution, and remained uninduced. Another 200 μL of culture was added to 5 mL of LB broth with 5 μL of kanamycin sulfate, and was induced with 5 μL of 0.5 M IPTG after reaching an OD of 0.8, and incubated further for 4 hours at 37° C. The induced and uninduced E. coli were lysed using Bug Buster Protein Extraction Agent (Millipore Sigma) and centrifuged to separate the soluble proteins and the lysate. The soluble proteins were prepared for SDS-PAGE by diluting 18 μL of sample in 6 μL Laemmli buffer (BioRad) supplemented with 10 v/v % β-mercaptoethanol (BioRad) and heated for 5 minutes at 90° C. The sampled were loaded into a 4-20% Mini-PROTEAN® TGX™ Precast Protein Gel (BioRad), run under denaturing conditions at 200 V for 35 minutes, stained with Coomassie blue dye, and imaged on an Azure 200 Gel Imager (Azure Biosystems, Inc.).

### Collection and Purification of Soluble Fusion Proteins

Transformed E. coli were grown in 20 mL LB broth supplemented with kanamycin to a 0.5 mM concentration and incubated overnight at 37° C. The contents were then transferred into 1.8 L of Terrific Broth (TB) supplemented with kanamycin to 0.5 mM and 500 μL of anti-foam 204 (Thermo Scientific) and cultured at 37° C. in a LEX-10 bioreactor (Epiphyte3). When the OD600 reached approximately 1.4, 1.8 mL of 0.5 M IPTG was added to the growth vessel to obtain a final concentration of 0.5 μM, and the temperature was reduced to 18° C. for 18 hours for induction of protein expression. After 18 hours, the culture was centrifuged for 20 minutes at 4° C. at 6000 RPM, and the cell pellet was removed and transferred to two 50 mL conical tubes. Binding buffer (50 mL of 1 M tris pH 7.5, 100 mL of 5 M NaCl, 5 mL of 1 M imidazole, and 845 mL RO water) was added to the cell pellet to a volume of 35 mL and lysed using a probe sonicator (Fisher Scientific) at 55% output for 10 sec on/10 sec off for 6.5 minutes in an ice bath. The sonicated product was centrifuged at 13,000 rcf for 30 minutes at 4° C. The supernatant was transferred to a 50 mL conical tubes along with 1.8 mL of Cobalt-NTA Agarose Beads (GoldBio; Cobalt beads) and rotated at 4° C. for 45 minutes. The supernatant was then transferred to a Econo-Column® chromatograph column (Biorad), washed with 50 mL of wash buffer (50 mL of 1 M tris pH 7.5, 100 mL of 5 M NaCl, 30 mL of 1 M imidazole, and 820 mL RO water) and eluted into 10 mL of elution buffer (50 mL of 1 M tris pH 7.5, 100 mL of 5 M NaCl, 250 mL of 1 M imidazole, and 600 mL RO water). The collected solution was then buffer-exchanged into 0.5 M Tris pH 8 using a 3 kDa molecular weight cut-off (MWCO) centrifuge filter (Millipore) and frozen at −80° C. until further use. SDS-PAGE was used to determine purity of the affibody at each step. UV-vis spectroscopy (Implen NP80) at 280 nm was used to determine the final concentration of the affibodies.

### Collection and Purification of Insoluble Fusion Proteins

Transformed E. coli were grown in 20 mL LB broth supplemented with kanamycin to a 0.5 mM concentration and incubated overnight at 37° C. The contents were then transferred into 1.8 L of Terrific Broth (TB) supplemented with kanamycin to 0.5 mM and 500 μL of anti-foam 204 (Thermo Scientific) and cultured at 37° C. in a LEX-10 bioreactor (Epiphyte3). When the OD600 reached approximately 1.4, 1.8 mL of 0.5 M IPTG was added to the growth vessel to obtain a final concentration of 0.5 μM, and the temperature was reduced to 18° C. for 18 hours for induction of protein expression. After 18 hours, the culture was centrifuged for 20 minutes at 4° C. at 6000 RPM, and the cell pellet was removed and transferred to two 50 mL conical tubes. The collected cell pellet was resuspended completely in 10 ml of pH 8 buffer (100 mM monobasic sodium phosphate, 10 mM Tris, and 8M Urea). The lysate was then incubated at room temperature for 30 min. After 30 minutes, the lysate was centrifuged at 10000 g for 30 minutes at room temperature. The supernatant was collected and combined with 1 ml of Cobalt-NTA Agarose Beads (GoldBio; Cobalt beads). The lysate-bead mixture was rotated for 1 hour in a rotisserie at room temperature. After an hour, the binding suspension was transferred to a Econo-Column® chromatograph column (Biorad) with a capped bottom outlet. The bottom cap of the column was removed to collect the flow-through. The column was washed with 5 mL of pH 6.3 buffer (100 mM monobasic sodium phosphate, 10 mM Tris, and 8 M Urea). The fusion protein was finally eluted five times each using 0.5 mL of pH 4.5 buffer (100 mM monobasic sodium phosphate, 10 mM Tris, and 8 M Urea). Each eluate fraction was collected in a separate tube and the protein concentration of each fraction was measured using the IMPLEN spectrophotometer. Following protein collection, the fusion proteins underwent stepwise dialysis to buffer exchange the protein from 8 M urea into 1×PBS. The eluted protein was added to dialysis tubing (6-8 kDa VWR) and transferred to a 1 L solution of 8 M urea. The protein was dialyzed for one hour before replacing 500 mL of the volume with 500 mL of 4 M urea solution. After 1 hour had passed, another half of the volume was replaced with another 500 mL of 4M Urea. The same solution replacement occurred for 2 M Urea, 1 M Urea, and 1 M PBS solution until the protein was dialyzed in 1 M PBS solution for 1 hour. The total time for dialysis is around 7-8 hours. The final protein was transferred out of the dialysis tubing into centrifuge tubes to be stored at −80° C. after measuring protein concentration using UV-vis spectrophotometry.

### Circular Dichroism of Soluble Affibodies

A Jasco J-815 circular dichroism spectropolarimeter (CD Spec; JASCO) was used to characterize the secondary protein structure of the affibodies. Purified affibody was buffer exchanged into PBS pH 6.92 using Zeba columns and diluted to a concentration below 50 PM. The protein was loaded into a quartz cuvette with 1 mm path length and placed in the CD Spec. The circular dichroism value and the high-tension voltage were collected over a wavelength range of 190-250 nm. The circular dichroism was converted to molar ellipticity using the molecular weight and concentration of each affibody.

### Native Ion Mass Spectrometry of Soluble Affibodies

A Waters Synapt G2Si mass spectrometer, calibrated with CsI cluster ions, was used to characterize the purity and mass of the collected affibodies. For each affibody, 1 mL of approximately 0.1 mg mL⁻¹ was buffer-exchanged into 200 mM ammonium acetate pH 7.52 via 6 kDa molecular weight cut-off Micro Bio-Spin 6 Columns (BioRad) and diluted to approximately 20 μM. Mass spectra were collected over 1-5 minutes using nano-electrospray ionization at a capillary voltage of 0.7-1.0 kV. Samples were deconvolved in UniDec¹¹⁵ using charge states 3 to 7 and an output mass range of 5,000-9,000 Da.

### Characterizing Binding Interactions of Soluble Affibodies via BioLayer Interferometry

The binding interaction between BMP-2 and each soluble affibody was measured using a Gator® Plus biolayer interferometer (BLI; Gator Bio). Biotinylated BMP-2 was buffer-exchanged into PBS and diluted to 25 nM in PBS with 0.05% Tween20 (PBST; Thermo Fisher). Each soluble affibody was also buffer-exchanged into PBS and diluted to concentrations between 0-125 nM in PBST. Streptavidin-coated BLI probes (Gator Bio) were pre-soaked in 250 μL PBST for 45 minutes. The probes were then baselined with 200 μL PBST for 300 seconds and loaded with bBMP-2 for 90 seconds or until the wavelength shift plateaued. A new baseline was established using 200 μL PBST for 90 seconds, followed by 300 seconds of association with 200 μL of the various concentrations of affibody and dissociation for 300 seconds in 200 μL PBST. The association and dissociation data for the first 120 seconds were used to avoid confounding nonspecific binding interactions. One probe was loaded with bBMP-2 and no affibodies and another probe was loaded with 125 nM of affibody and no bBMP-2 for use as a reference probe and to quantify nonspecific binding to the probes, respectively.

### Circular Dichroism of Soluble Fusion Proteins

A Jasco J-815 circular dichroism spectropolarimeter (CD Spec; JASCO) was used to characterize the secondary protein structure of the fusion proteins. Purified fusion protein was buffer exchanged into 10 mM Tris using Zeba columns and diluted to a concentration below 30 PM. The protein was loaded into a quartz cuvette with 1 mm path length and placed in the CD Spec. The circular dichroism value and the high-tension voltage were collected over a wavelength range of 190-250 nm. The circular dichroism signal was converted to molar ellipticity using the molecular weight and concentration of each fusion protein.

### Native Ion Mass Spectrometry of Soluble Fusion Proteins

A Waters Synapt G2Si mass spectrometer, calibrated with CsI cluster ions, was used to characterize the purity and mass of the collected fusion proteins. For each fusion protein, 1 mL of approximately 0.1 mg mL⁻¹ was buffer-exchanged into 200 mM ammonium acetate pH 7.52 via 6 kDa molecular weight cut-off Micro Bio-Spin 6 Columns (BioRad) and diluted to approximately 20 μM. Mass spectra were collected over 1-5 minutes using nano-electrospray ionization at a capillary voltage of 0.7-1.0 kV. Samples were deconvolved in UniDec¹¹⁵ using charge states 3 to 7 and an output mass range of 5,000-10,000 Da.

### Characterizing Binding Interactions of Soluble Collagen-Binding Fusion Proteins via Biolayer Interferometry

The binding interaction between BMP-2 and each collagen-binding fusion protein was measured using a Gator® Plus biolayer interferometer (BLI; Gator Bio). Biotinylated BMP-2 was buffer-exchanged into PBS and diluted to 25 nM in PBS with 0.05% Tween20 (PBST; Thermo Fisher). Each soluble affibody was also buffer-exchanged into 1×PBS and diluted to concentrations between 0-500 nM in PBST. Streptavidin-coated BLI probes (Gator Bio) were pre-soaked in 250 μL PBST for 45 minutes. The probes were then baselined with 200 μL PBST for 300 seconds and loaded with bBMP-2 for 500 seconds or until the wavelength shift plateaued. A new baseline was established using 200 μL PBST for 300 seconds, followed by 1200 seconds of association with 200 μL of the various concentrations of collagen-binding fusion protein and dissociation for 1200 seconds in 200 μL PBST. The association and dissociation data for the first 120 seconds were used to avoid confounding nonspecific binding interactions. One probe was loaded with 25 nM bBMP-2 and no fusion protein and another probe was loaded with 62.5 nM of fusion protein and no bBMP-2 for use as a reference probe and to quantify nonspecific binding to the probes, respectively.

### Computational Prediction of Binding Interaction Between BMP-2 and Affibodies

The high- and low-affinity affibody sequences were input into AlphaFold2,⁶⁹ which outputs high-ranking protein structures and their corresponding prediction confidence as determined by AlphaFold2's deep learning network. The five highest ranked affibody structure prediction models for each unique affibody sequence were energetically minimized using Rosetta build 314.^{64-67,69-72} Specifically, a full-atom refinement application called Relax was used, which samples backbone and sidechain conformations to make local optimizations to the protein structure based on physics and heuristics-based weighted calculations.^{71,76,116} Furthermore, the Relax protocol constrains the minimization movements to input structure, thereby biasing the refinements to the AlphaFold2 structure predictions. The affibody binding sites and orientations to target protein BMP-2 (PDB ID: 3BMP) were then modeled using the publicly available web server for ZDOCK, a docking application which approximates global binding.⁷³ The ten most probable affibody-BMP-2 complexes determined in the ZDOCK 3.0.2 algorithm for each sequence were similarly relaxed with Rosetta. To characterize the predicted interactions from ZDOCK, we performed interface analysis using PyMOL.⁷⁴ The x-ray crystallography structure of BMP Receptor Type-1A was used as present in the RSCB protein databank (PDB: 1REW). The AlphaFold2-Multimer tool was also used to predict the docking of BMP Receptor II (BMPR-JJ) using PDB: 7PPA onto BMP-2 using PDB: 3BMP.117,118

### Computational Prediction of Binding Interaction Between BMP-2 and Fusion Proteins

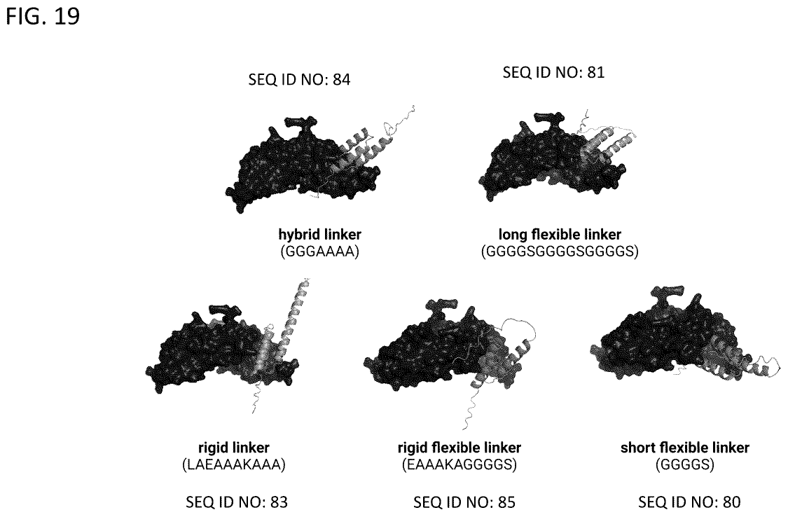

The amino acid sequences of the fusion proteins with different linkers were input into AlphaFold2, which outputs the most probable protein structures and their corresponding prediction confidence as determined by AlphaFold2's deep learning network. The affibody binding to the target protein BMP-2 (PDB ID: 3BMP) was then modeled using the publicly available web server for ZDOCK, a protein docking application which approximates global binding. The most probable fusion protein-BMP-2 complex determined in the ZDOCK 3.0.2 algorithm for each sequence was visualized in PyMOL to determine which linker sequence provided the greatest available binding interface for the fusion protein to simultaneously bind to BMP-2 and collagen (FIG. 19). To characterize the predicted interactions from ZDOCK, we performed interface analysis using PyMOL.

### Cell Culture

High glucose Dulbecco's modified eagle medium (DMEM; Gibco) was supplemented with fetal bovine serum (FBS; Bio-techne) to either 1 v/v % or 10 v/v % to create low serum or high serum medium, respectively. Both media were supplemented with 1 mL of penicillin-streptomycin solution (Millipore Sigma) containing 10,000 U mL⁻¹ penicillin and 10,000 pg mL⁻¹ streptomycin. C2C12 immortalized murine skeletal myoblasts (CRL-1772; ATCC) were maintained in high serum medium, detached and passaged using 0.25% trypsin-EDTA (Lonza), and reseeded into T75 flasks (NEST Scientific) at a density of 2,500 cells cm⁻². Cell number and viability were quantified using a Countess II Automated Cell Counter (Invitrogen).

### C2C12 Cytocompatibility Assay for Affibodies

C2C12 myoblasts were seeded onto a 96-well plate at a concentration of 2000 cells cm⁻² in 180 μL of high serum medium and allowed to adhere for 6 h. Affibodies were buffer-exchanged into PBS using 7 kDa Zeba columns, sterile-filtered through 0.22 μm filters, and diluted in sterile Dulbecco's PBS. Affibodies were added to the cell culture wells at final concentrations of 10 nM, 20 nM, 40 nM, 80 nM, or 800 nM and incubated for 72 h. The cells were washed with PBS and stained for 30 minutes at 37° C. with fresh high serum medium containing 4 mM Calcein AM (Fisher Scientific) and 2 mM ethidium homodimer-1 (Santa Cruz Biotechnology) to quantify the number of live and dead cells, respectively. Cells were imaged using a LionHeart FX automated microscope (BioTek). The number of live and dead cells were quantified using a custom script developed for Python. Cell viability was calculated by dividing the number of living cells by the total number of cells.

### C2C12 Alkaline Phosphatase Activity Assay

C2C12 myoblasts were seeded onto a 96-well plate at a concentration of 62,500 cells cm⁻² in 200 μL of high serum medium and allowed to adhere for 6 h, after which the cells were washed with PBS and resuspended in 100 μL of low serum media containing the different treatments. Affibodies were buffer-exchanged into PBS using 7 kDa Zeba columns, sterile-filtered through 0.22 μm filters, and diluted in PBS to concentrations of 10 nM, 20 nM, 40 nM, 80 nM, and 1000 nM. Sterile carrier-free recombinant human BMP-2 (R&D Biosystems) was diluted to 20 nM in PBS. 20 nM of BMP-2 and/or 10 nM, 20 nM, 40 nM, 80 nM, or 1000 nM of affibodies were added sequentially for the “uncomplexed” treatment groups or as a premixed solution for the “complexed” treatment groups as described above. After 72 h, the cells were lysed with Cellytic M (Millipore Sigma), and their ALP activity was quantified and normalized to the total amount of double stranded DNA (dsDNA) present in each well.⁸⁸ For the ALP colorimetric assay, 50 μL buffer solution consisting of equal volumes of 1.5 M 2-amino-2methyl-1-propanol solution pH 10.25, 20 mM p-nitrophenyl phosphate solution, and 10 mM MgCl₂ hexahydrate solution were mixed with 50 μL of lysed cells and incubated in the dark for 20 minutes before the absorbance of the solutions was read at 405 nm (Synergy Neo2, Biotek). The colorimetric change in the solutions was converted to p-nitrophenol concentration using a calibration curve of 0-0.8 μmol mL⁻¹ 4-nitrophenol solution (Millipore Sigma). The QuantiFluor dsDNA System (Promega) was used for dsDNA quantification. The ALP activity in each well was normalized to the dsDNA content to account for variability in cell number between samples.

### Statistical Analysis

Data pre-processing was performed using GraphPad Prism 9.5.1, except flow cytometry data which was prepared using FlowJo 10.8.1, biolayer interferometry preparation and curve fitting which was prepared using GatorOne 2.10, and protein structural presentation which was prepared using PyMOL 4.6.0. All relevant data are reported as means+/−standard deviation with sample sizes indicated in the figure caption. All statistical methods used to assess significant differences and applicable post-hoc tests are reported in figure descriptions. For all data, the use of one-way ANOVA was chosen for a single variable of comparison. For all two-way ANOVA, multiple variables were compared. Tukey post-hoc tests were performed to compare the significance of all groups between each other. Dunnett post-hoc tests were performed to compare the significance of all data to a control group.

### Example 2: Magnetic-Activated Cell Sorting Depleted Over 99% of the Yeast Display Library Diversity

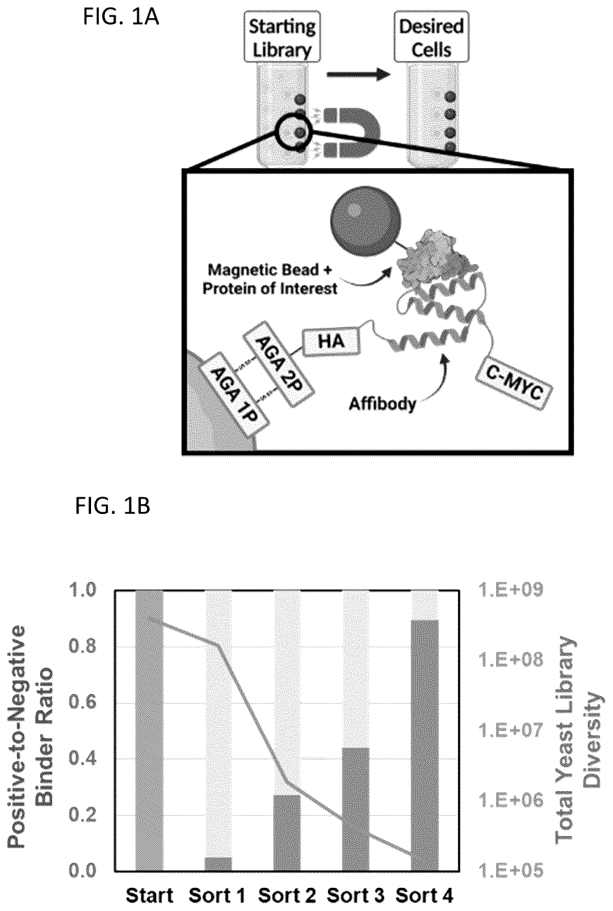

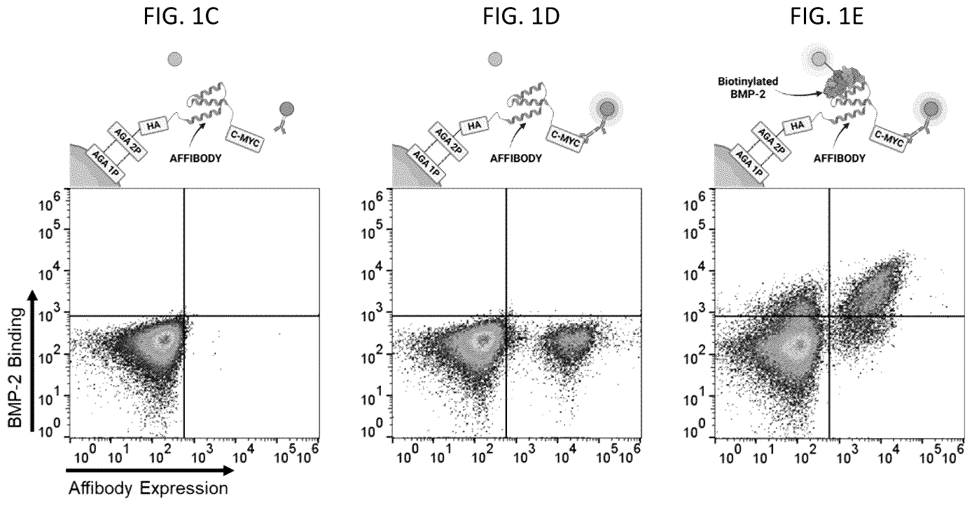

Four rounds of magnetic-activated cell sorting (MACS) were performed to enrich for BMP-2-binding affibodies within the yeast surface display library (FIG. 1A). Each round of MACS consisted of two negative magnetic bead sorts to remove non-specific protein binders^{39,40,50,51} and one positive magnetic bead sort using beads conjugated with BMP-2 to enrich for yeast displaying BMP-2-specific affibodies. Following each round of MACS, yeast from each bead sort were plated on selective growth plates to count the number of colonies that bound to the negative beads and BMP-2-conjugated beads.

The new yeast library diversity after each round of MACS was estimated by counting the number of colonies grown on the BMP-2 plates, while the ratio of positive-to-negative binders was calculated by dividing the number of colonies grown on the BMP-2 plates by the sum of colonies grown on the negative plates. FIG. 1B demonstrates how each round of MACS enriched BMP-2 specific affibodies (blue bar graph) while reducing the total yeast library diversity (i.e., number of unique variants) (orange line plot). After four rounds of MACS, the library diversity was reduced by over 99%, and the number of BMP-2-specific affibodies was estimated to account for approximately 89% of the remaining library.

### Example 3: Fluorescence-Activated Cell Sorting Identified BMP-2-Specific Affibodies

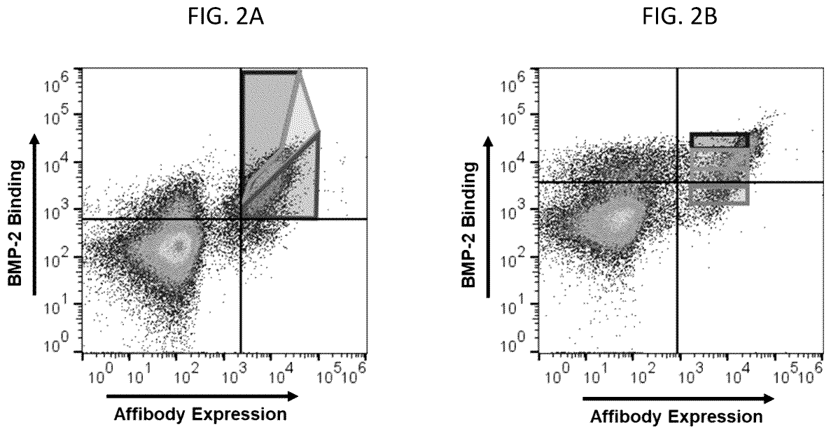

Following four rounds of MACS, fluorescence-activated cell sorting (FACS) was performed on the enriched yeast library, which was gated into populations corresponding to different affinity ranges for BMP-2 binding. Yeast were incubated in 0.1 mg mL⁻¹ bovine serum albumin (BSA) in phosphate buffered saline (PBS) (i.e., PBSA) without fluorescent tags or proteins (cells-only control) (FIG. 1C), with a mouse anti-c-myc antibody (αCMYC, 9E10) to assess affibody expression levels by binding to the N-terminal c-myc epitope (FIG. 1D), or with both αCMYC and biotinylated BMP-2 (bBMP) to assess BMP-2 binding to displayed affibodies (FIG. 1E). Except for the cells-only control, all yeast were incubated with secondary fluorescent tags that bound specifically to αCMYC (AlexaFluor 647 goat anti-mouse conjugate; AF647) or bBMP (AlexaFluor 488 streptavidin conjugate; AF488). AF647+/AF488+ yeast cells were gated using two gating approaches and collected (FIGS. 2A-2B). At least 10,000 yeast cells were collected from each gate during FACS to capture all unique affibody sequences from each gate.

Following FACS, yeast from each gate were plated onto selective growth plates and allowed to form discernable colonies that each contained a single affibody sequence (i.e., monoclonal yeast). Three colonies from each gate were grown in growth media for a total of 21 yeast clones. Sanger sequencing of plasmid DNA revealed 11 unique affibody sequences (SEQ ID NOS: 1 to 11).

### Example 4: Characterization of BMP-2 Binding to Monoclonal Yeast Affibodies

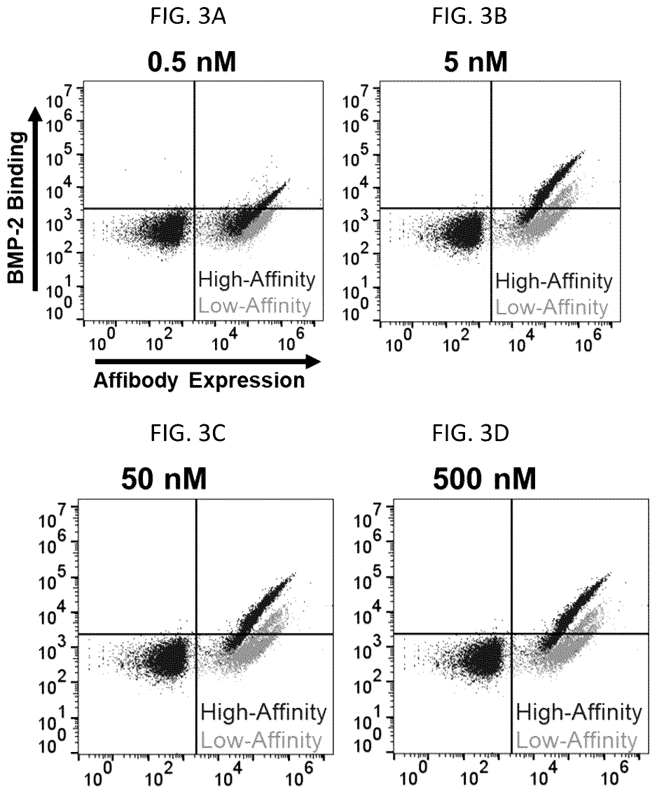

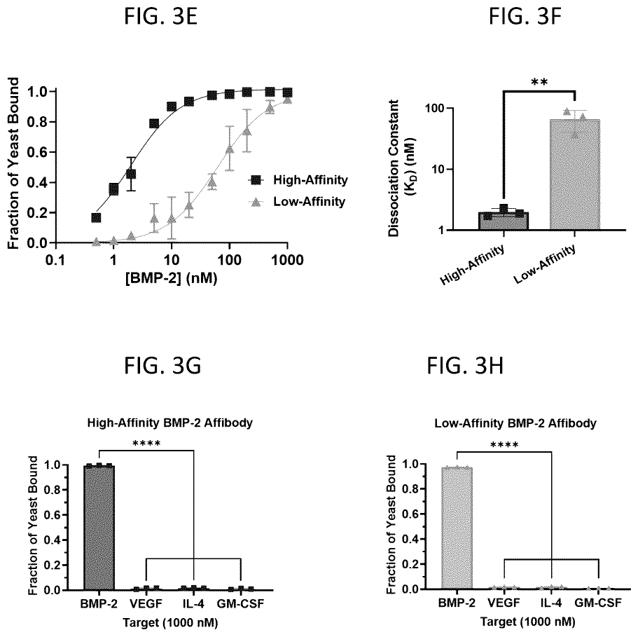

Binding affinities between BMP-2 and BMP-2-specific affibodies were assessed on yeast using flow cytometry. Similar to FACS, monoclonal affibody-displaying yeast were incubated in either PBSA, αCMYC and secondary solution, or αCMYC with a range of bBMP concentrations (0.5-1000 nM) and secondary solution (AF647 and AF488 for affibody expression and bBMP-2 binding, respectively). At each concentration of bBMP, the fraction of displayed affibodies that were bound to BMP-2 was determined by dividing the top right quadrant (AF647+/AF488+) by the right half of the graph (AF647+). With increasing bBMP-2 concentrations, more cells were labeled with AF488, resulting in an upward shift of the population that indicated increased bBMP-2 binding (FIGS. 3A-3D). Binding affinity was assessed by plotting the fraction of bBMP-2 bound over the bBMP-2 concentration range (FIG. 3E). Monoclonal yeast that demonstrated a greater bBMP-2 binding had higher affinities for BMP-2.⁵² Equilibrium dissociation constants (K_{D}) were calculated by performing a nonlinear regression on bBMP-2 binding to affibody-displaying yeast at varying bBMP-2 concentrations (FIGS. 3E-3F).

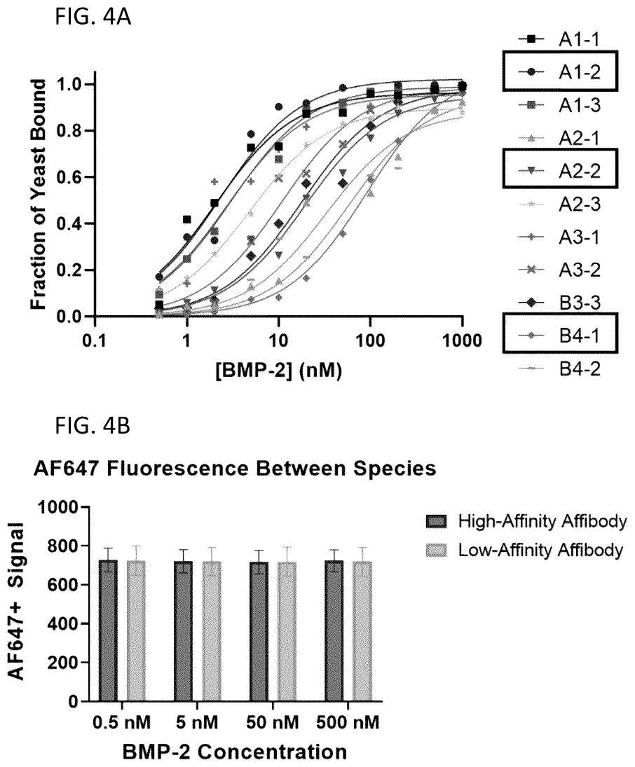

The affinities of all 11 unique clones that bound to BMP-2 (SEQ ID NOS: 1-11) were quantified (FIG. 4A) and two unique clones (SEQ ID NOS: 1 and 3) were chosen for further examination. These clones displayed significantly different affinities for BMP-2 and will be identified hereafter as high-affinity (K_{D}=1.95±0.14 nM) and low-affinity (K_{D}=61.82±9.38 nM) BMP-2-binding affibodies. The affinities between the other affibodies and BMP-2 were found to be within the range of the equilibrium dissociation constants of the high- and low-affinity affibodies. The quantity of surface-displayed affibodies may have affected the perceived affinity between the affibodies and BMP-2 by altering the ratio between the proteins and the protein-binding partners.⁵² The average AF647+ fluorescence signal, indicative of affibody expression, was similar between the high- and low-affinity affibodies at each BMP-2 concentration, confirming a comparable number of affibodies displayed on the surface of each monoclonal yeast species (FIG. 4B).

Specificity of the high- and low-affinity affibodies for BMP-2 was also assessed using flow cytometry. Several other proteins involved in the bone healing cascade were chosen to investigate specificity of the BMP-2 affibodies. Monoclonal affibody-displaying yeast were incubated with PBSA, αCMYC and secondary solution, or αCMYC with 1000 nM of biotinylated vascular endothelial growth factor (bVEGF), biotinylated interleukin-4 (bIL-4), or biotinylated granulocyte-macrophage colony stimulated factor (bGM-CSF) and secondary solution. All affibodies exhibited negligible binding to bVEGF, bIL-4, and bGM-CSF, demonstrating that these affibodies were specific to BMP-2 (FIGS. 3G-3H).

### Example 5: Collection and Characterization BMP-2-Specific Affibodies

Sequences for the high- and low-affinity affibodies (SEQ ID NOS: 1 and 3) modified with a 6-histidine (His-tag) for protein collection^{39,53,54} and a N-terminal cysteine for bioconjugation⁵⁵⁻⁵⁷ were ligated into a pET28b+ expression vector, which was transformed into chemically competent BL21 E. coli for protein expression. Soluble protein was collected using benchtop immobilized metal affinity chromatography (IMAC) with cobalt-nitrilotriacetic acid beads.⁵³ Approximately 10 mg of pure soluble affibodies were collected from each liter of E. coli culture.

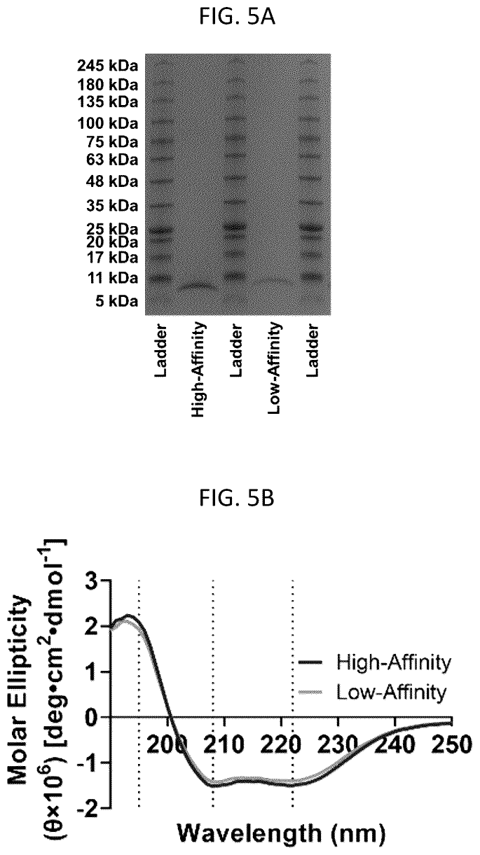

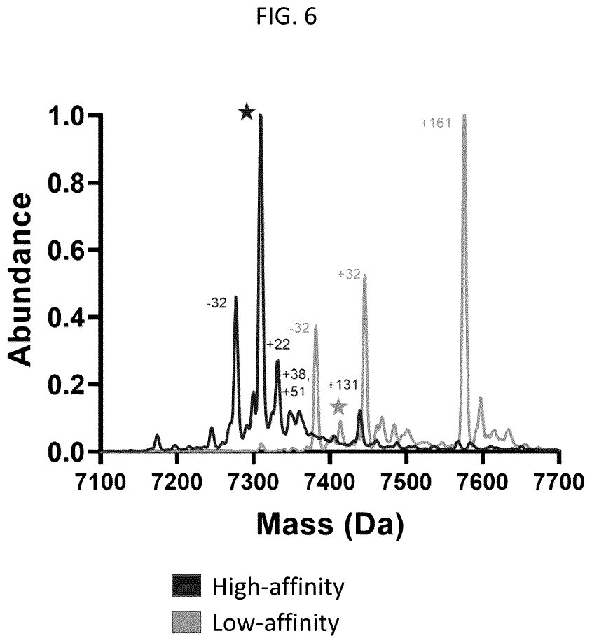

Sodium dodecyl sulfate polyacrylamide gel electrophoresis (SDS-PAGE) (FIG. 5A) and native ion mass spectrometry (NIMS) (FIG. 6) were used to determine the size of the affibodies. SDS-PAGE of purified affibodies (150 μM in tris, pH 8) revealed thick bands visible between the 5 kDa and 11 kDa rungs of the control ladder at the approximate expected sizes of the two affibodies (7308 Da and 7414 Da for the high- and low-affinity affibodies, respectively) without any other noticeable bands. Native ion mass spectrometry (NIMS) of affibodies (20 μM in 0.2 M ammonium acetate, pH 7.52) demonstrated a dominant high-affinity affibody peak corresponding to the expected mass of 7308 Da with other well-populated peaks associated with sodium and potassium adducts, the possible formation of a cysteic acid or piperidine on the C-terminal,⁵⁸ and glutamylation of the N-terminal cysteine. The low-affinity affibody (SEQ ID NO: 3) displayed a small peak at the expected mass of 7414 Da and had prominent peak shifts associated with the formation of a dehydroalanine,⁵⁹ a 32 Da shift attributed to a trisulfide bond,⁶⁰ an additional shift associated with a piperidine formation on the terminal cysteine,⁵⁸ and another prominent peak shift (161 Da) attributed to a carboxymethyl cysteine or carboxymethyl cystenyl.⁶¹ These data indicate that these affibodies may readily undergo post translational modifications and that the low affinity affibody may undergo extensive post-translational modification. However, these modifications mainly affect the terminal cysteine, which may affect chemical conjugation of the affibody to biomaterials, but are not expected to affect affibody binding affinity for BMP-2.⁵⁸

Circular dichroism was used to determine the secondary structure of the affibodies.⁶² Affibodies were diluted to concentrations between 17-30 μM in 5 mM tris pH 6.92, which was a pH equidistant from each of their isoelectric points. Both affibodies exhibited characteristic α-helical profiles, including troughs at 208 nm and 222 nm and a peak at 195 nm,^{40,62} confirming the secondary structure of the affibodies in their soluble state (FIG. 5B).⁶³

### Example 6: Characterization of Soluble Affibody-BMP-2 Binding Interactions

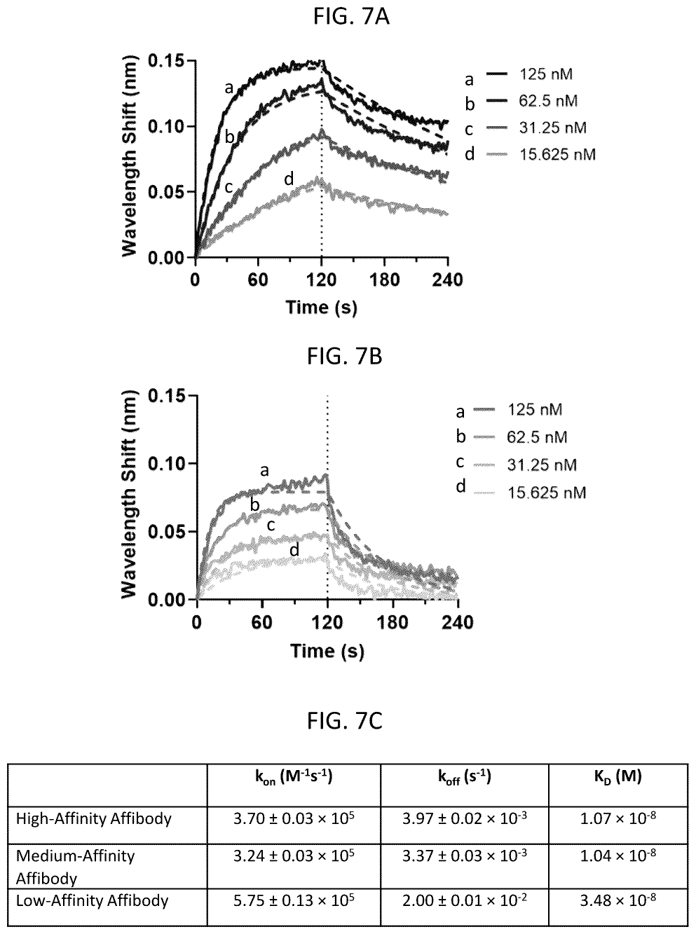

The binding interaction between the soluble high- (SEQ ID NO: 1) and low- (SEQ ID NO: 3) affinity affibodies and BMP-2 were characterized using biolayer interferometry (BLI). Streptavidin-coated BLI probes were coated with 25 nM bBMP-2 in PBS with 0.05% Tween-20® (PBST), followed by association of 0-125 nM of the purified soluble affibody in PBST for 120 seconds and dissociation in PBST for 120 seconds (FIGS. 7A-7B). BLI enabled the determination of the dissociation rate constant (k_{off}), association rate constant (kₒₙ), and overall equilibrium dissociation constant (K_{D}) of each binding interaction. The equilibrium dissociation constants of the high-, medium-, and low-affinity affibodies (SEQ ID NO: 1, 2 and 3, respectively) for BMP-2 were determined to be 10.7 nM, 10.4 nM, and 34.8 nM, respectively (FIG. 7C). The k_{off} of the low-affinity affibody was an order of magnitude higher than that of the high-affinity affibody, and the low-affinity affibody completely dissociated from the bBMP-2. While evaluating BMP-2-affibody binding on the surface of the yeast provided insight to the binding strength of the affibody, it was more representative of the avidity (i.e., total binding strength) rather than affinity of the individual affibodies and did not provide information about the association and dissociation rates of the binding interaction.⁵² As such, BLI provided a more representative measurement of affibody affinity for BMP-2 when integrated into hydrogels.

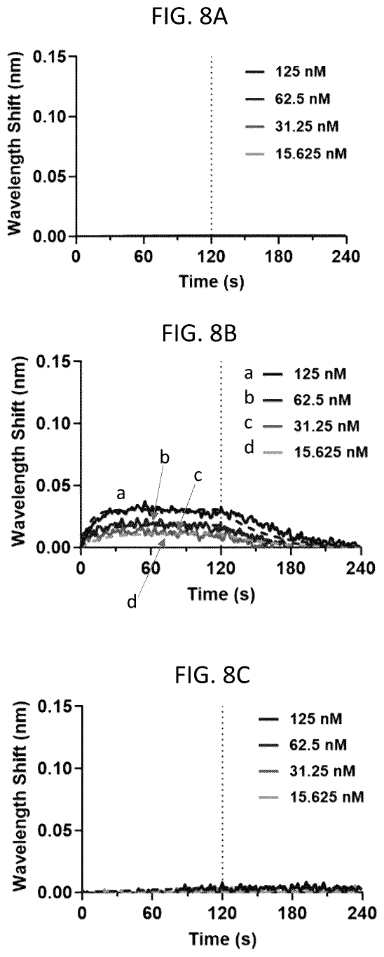

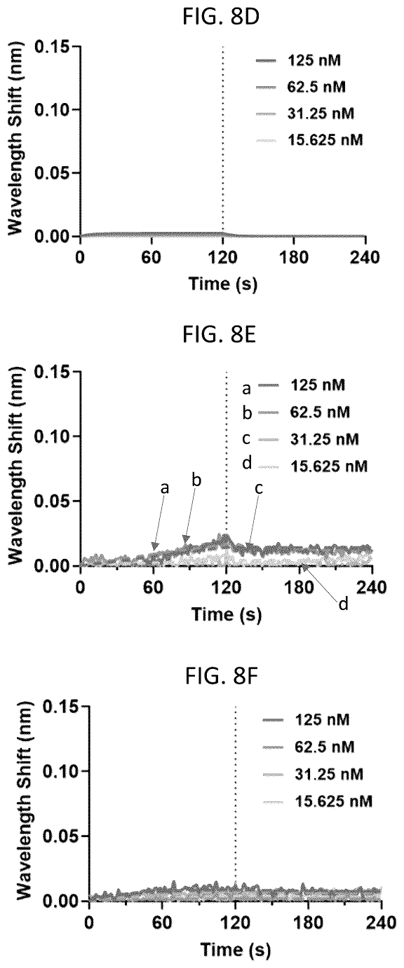

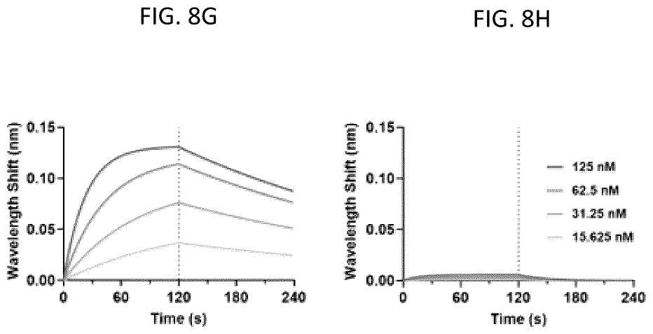

BLI was also performed using streptavidin-coated probes coated with 25 nM of bVEGF, bIL-4, or bGM-CSF followed by association and dissociation of 0-125 nM of high-, medium- and low-affinity affibody in PBST (FIGS. 8A-8H). There was no noticeable binding response to VEGF and GM-CSF, and only a minimal binding response to IL-4, indicating that the affibodies do not bind to VEGF or GM-CSF and bind minimally to IL-4 when compared to BMP-2. Overall, these results demonstrate that the soluble affibodies specifically bind to BMP-2 and that the high-affinity affibody (SEQ ID NO: 1) has a stronger interaction with BMP-2 compared to the low-affinity affibody (SEQ ID NO: 3).

### Example 7: Computational Predictions of Affibody Binding to BMP-2

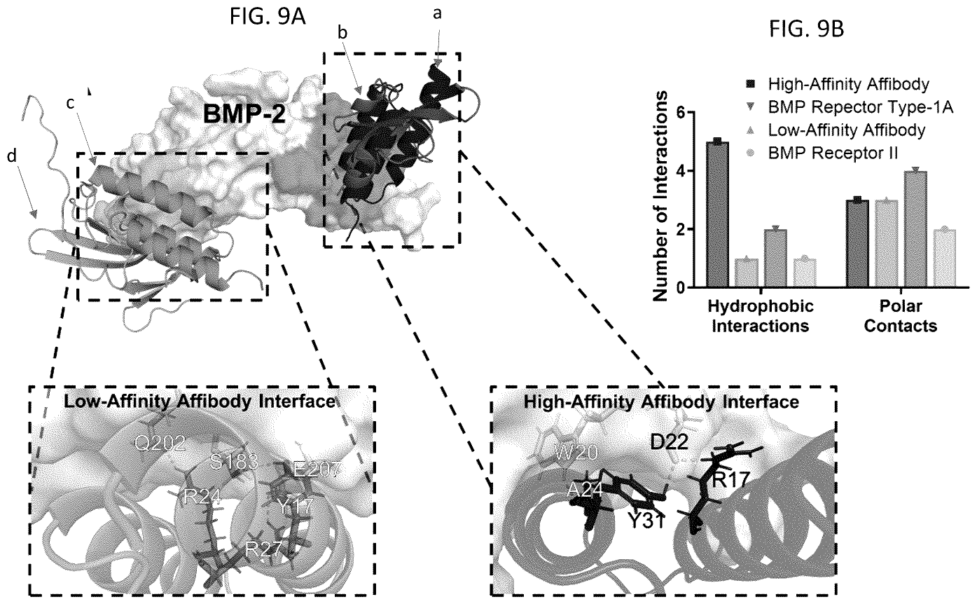

The computational tools AlphaFold 2, ZDOCK, and Rosetta were used to predict the site of interaction between each affibody and BMP-2. AlphaFold predicted the folded structures of the affibodies with high confidence (predicted local distance difference test score >95 for most of the predictions).⁶⁴⁻⁶⁹ Predicted affibody structures were energetically minimized using a protocol in Rosetta.⁷⁰⁻⁷² These structures were then docked to BMP-2 using the ZDOCK algorithm.⁷³ The top-ranked conformations for each affibody-BMP-2 complex were visualized in Pymol (FIG. 9A).⁷⁴ The high-affinity affibody was predicted to interact with BMP-2 at the binding site commonly referred to as the “wrist,” while the low-affinity affibody was predicted to bind to a different site of BMP-2 known as the “knuckle.”^{1,75} Electrostatic interactions were defined as polar contacts between BMP-2 and each respective affibody. Hydrophobic interactions were defined as hydrophobic amino acids of BMP-2 less than 3.5 Å away from each affibody, which structurally contributed to the formation of a hydrophobic pocket. Interfacial calculations suggested that the interactions between the high-affinity affibody and BMP-2 were governed by multiple hydrophobic and electrostatic intermolecular interactions with BMP-2 at the wrist binding site, whereas the low-affinity affibody interacted with the knuckle binding site of BMP-2 primarily through electrostatic interactions (FIG. 9B).^{76,77} In comparison to collagen which has been shown to have a >500 nM equilibrium dissociation constant with BMP-2,⁷⁸ the affibody-based electrostatic interactions with BMP-2 are an order of magnitude stronger. Furthermore, collagen interacts with many different biomolecules, making its interaction with BMP-2 less specific than the BMP-2-affibody interactions and potentially less controllable in complex in vivo environments.^{78,79}

The BMP-2 wrist binding epitope has been recognized as the binding site for BMP receptor type-1A (BMPR1A), with which the growth factor makes a relatively strong binding interaction (K_{D}≈0.7 nM),⁸⁰ while the knuckle is a relatively weak binding site for BMP receptor type II (BMPR-II) (K_{D}≈100 nM).^{1,81} BMP-2-induced osteogenesis occurs in skeletal myoblasts and mesenchymal stromal cells when a BMP-2 dimer interacts with a cell membrane-bound heterotetramer formed from two BMPR1A and two BMPR-II.^{1,75,82,83} These data indicate that binding of the affibody to BMP-2 at higher affinities than its receptors could potentially interfere with BMP-2-receptor binding, subsequently inhibiting BMP-2-induced osteogenesis. Additionally, increasing the quantity of affibody present in solution may shift the dynamics of receptor binding, resulting in a concentration-dependent inhibition. To test these hypotheses in vitro, C2C12 immortalized murine skeletal myoblast cell line were used. The murine cell line has been shown to express markers of early osteogenic differentiation, such as alkaline phosphate (ALP) activity, in the presence of BMP-2 in a dose-dependent manner.^{81,84,85}

### Example 8: Affibodies Do Not Impact C2C12 Cell Viability or Proliferation

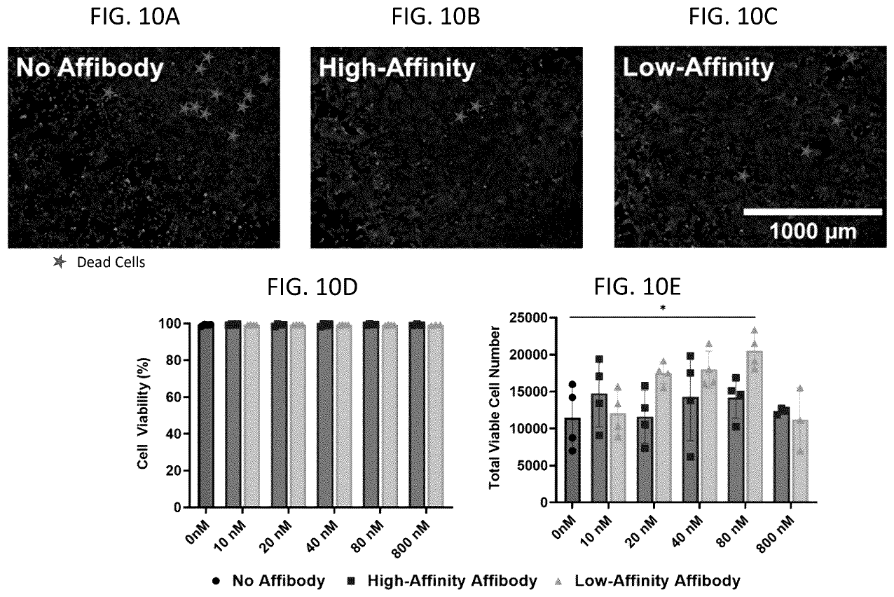

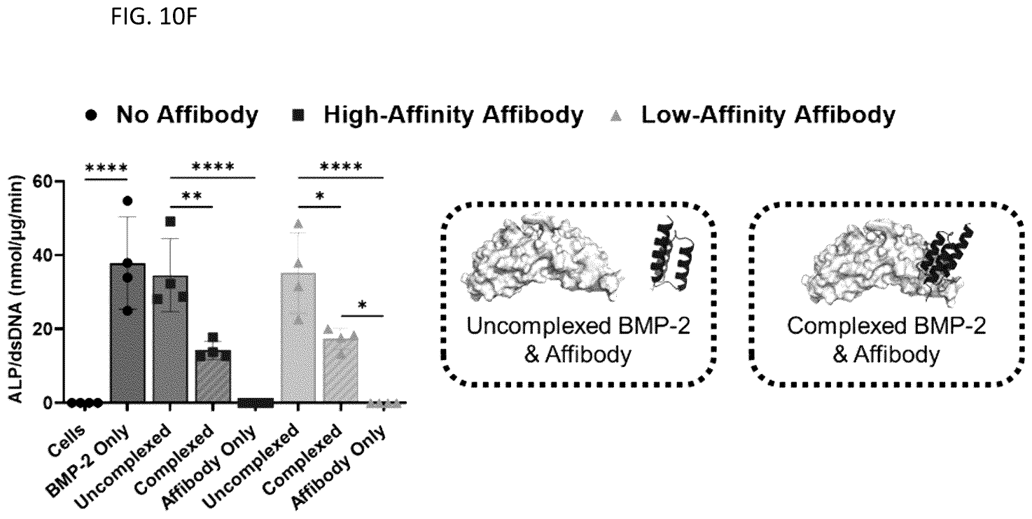

The cytocompatibility of the soluble BMP-2-specific affibodies was assessed using C2C12 cells. Soluble high- and low-affinity affibodies (SEQ ID NO: 1 and 3, respectively) were added to C2C12 cultures at final concentrations of 10 nM, 20 nM, 40 nM, 80 nM, or 800 nM. After incubation for 72 hours, cells were stained with calcein AM and ethidium homodimer-1 to quantify live and dead cells, respectively, and imaged (FIGS. 10A-10C). Cell viability was calculated by dividing the number of living cells by the total number of cells (FIG. 10D), and total cell count was calculated by averaging the number of live cells in each image (FIG. 10E). C2C12 viability was not negatively impacted by the introduction of any concentration of affibodies. The total viable cell count was also largely unaffected by the various concentrations of the affibodies. Although treatment with 80 nM of low affinity affibody increased cell number, the lack of discernable pattern indicated that cell proliferation was not affected by the affibodies.

### Example 9: Affibody-BMP-2 Binding Reduces Alkaline Phosphatase Activity of C2C12 Cells

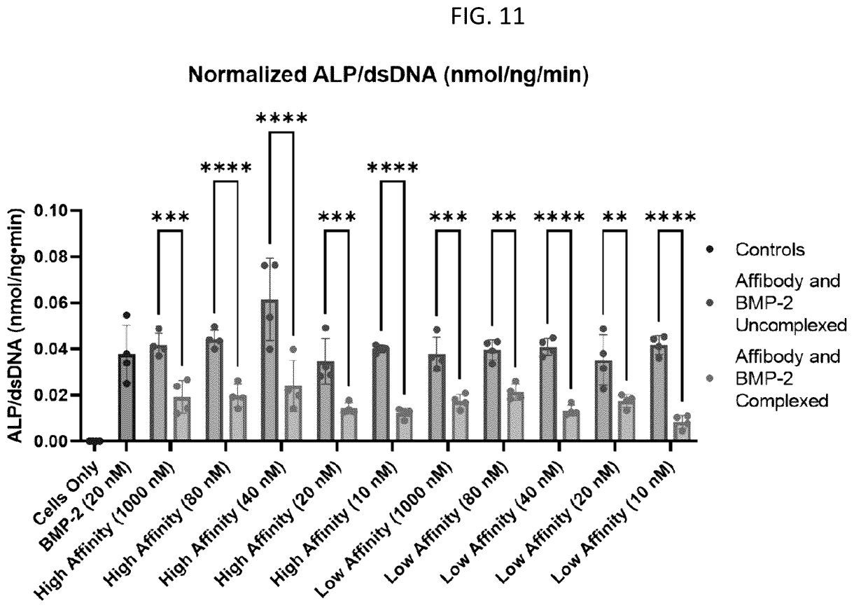

ALP activity, which is an indicator of early osteogenic differentiation,⁸⁶⁻⁸⁷ was used to assess the impact of affibodies on BMP-2 bioactivity. 20 nM of BMP-2 with or without different concentrations of soluble high- or low-affinity affibodies were added to C2C12 cultures sequentially for the “uncomplexed” treatment groups (affibodies first, incubated for 45 minutes, followed by BMP-2) or as a premixed solution (45 minutes of mixing to ensure adequate time for interaction) for the “complexed” treatment groups. After 72 hours, cells were lysed and their ALP activity was quantified by a colorimetric change caused by the ALP-induced catalysis of p-nitro phenyl phosphate to p-nitrophenol.⁸⁸ ALP activity was normalized to the total amount of double-stranded DNA present in each cell culture. Treatment with both uncomplexed and complexed BMP-2 and affibodies was performed to compare the different states in which BMP-2 may be presented in clinical applications. Traditionally, BMP-2 is soaked into a collagen sponge prior to delivery to a bone defect,⁸⁹ resulting in some burst release of protein and some long-term retention of BMP-2 within the scaffold. In the absence of a hydrogel or other delivery vehicle, the uncomplexed treatment group represented the released BMP-2, which would interact with cells outside of the scaffold, while the complexed treatment group represented the BMP-2 that would remain bound to the affibodies within the delivery vehicle and would interact with cells that migrate into the scaffold.^{31,90} FIG. 10F depicts the normalized ALP activity for the experimental groups using 20 nM affibody concentrations (1:1 ratio of BMP-2 to affibody). FIG. 11 depicts additional affibody concentrations (10⁻¹⁰⁰⁰ nM), which resulted in ALP activity that followed similar trends. No significant differences were observed between ALP activity induced by high- and low-affinity affibody-BMP-2 treatment groups. In the absence of BMP-2, no ALP activity was observed, regardless of affibody presence. In the presence of BMP-2 alone, normalized ALP activity was 37.9±12.5 nmol pNPP μg⁻¹ dsDNA min⁻¹, indicative of early osteogenic differentiation of the C2C12 cells.⁸⁴ For the uncomplexed approach, the affibodies caused an insignificant reduction in ALP activity. For all complexed treatment groups, ALP activity was significantly reduced compared to BMP-2 treatment and the uncomplexed treatment groups. These results indicate that affibody binding to BMP-2 may inhibit some of the function of BMP-2 or inhibited its interaction with the requisite tetramer complex of membrane-bound BMP receptors,^{1,75} supporting the computational docking simulations. The BLI data (FIGS. 8A-8B) indicates that some portion of the BMP-2 remained bound to the high-affinity affibody for an extended period, potentially exceeding the 72 hours incubation period. Even though the BLI data indicated that the low-affinity binder allows for complete dissociation of the BMP-2, the low-affinity binder has a greater affinity for the knuckle than BMPR-II. Since optimal BMP-2 activity requires a tetramer of two BMPR-1A and two BMPR-II,⁸¹ disruptions to the binding interactions between BMP-2 and either BMP receptor on the C2C12 cells may have restricted the induction of ALP activity in C2C12 cells.

### Example 10: Identification of GM-CSF Affibodies

Using the methods described in Example 1, affibodies were identified for GM-CSF.

An initial yeast library expressing millions of randomized affibody variants underwent four cycles of magnetic-activated cell sorting to enrich the library for GM-CSF binders, followed by two cycles of fluorescence-activated cell sorting to isolate yeast populations that bind to GM-CSF. Individual yeast clones were sequenced, and their binding affinities were characterized.

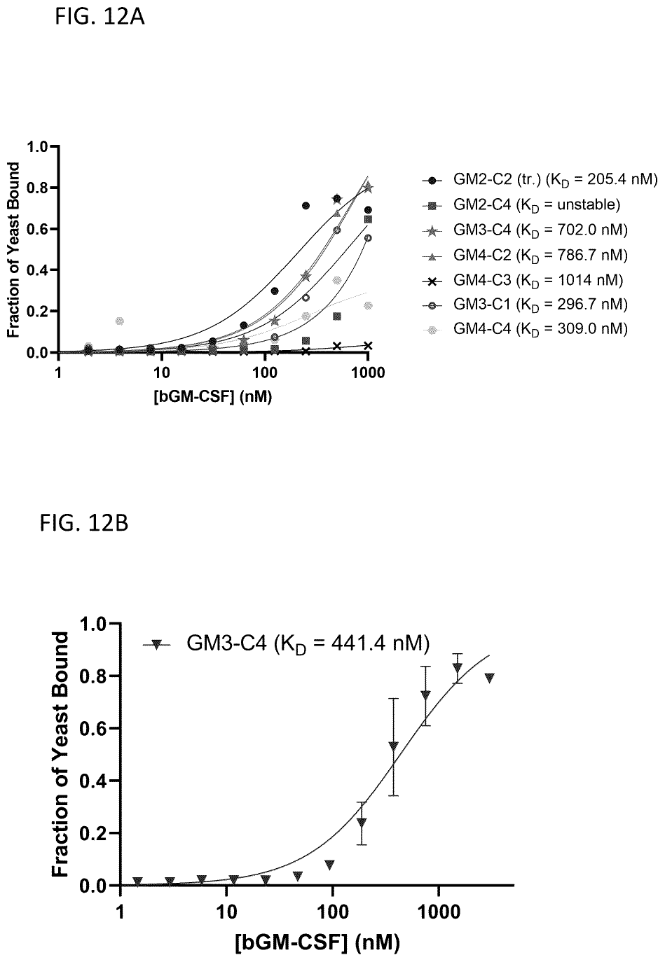

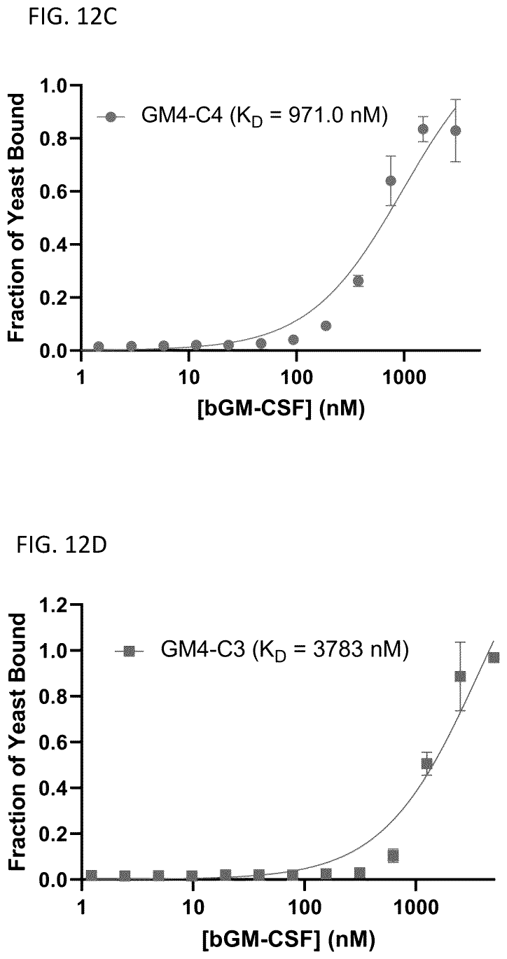

As shown in FIGS. 12A-12D, several affibodies specific for granulocyte macrophage colony-stimulating factor (GM-CSF) were identified (SEQ ID NOs: 12-19), having a dissociation constant of about 205.4 to 786.7 nM. Such affibodies can be linked to a localization domain to form dual-affinity fusion proteins, which can be used to control the release of GM-CSF. For example, a medical material can be loaded with one or more dual-affinity fusion proteins bound to GM-CSF, and when the medical material is applied to a subject to treat a wound, and/or a bone or cartilage injury or disease (for example by applying the medical material to a wound or injury site), the dual affinity fusion proteins will control the release of GM-CSF.

### Example 11: Identification of Affibodies for Angiogenesis

Using the methods described in Example 1, affibodies were identified for proteins associated with angiogenesis.

A yeast surface display library containing approximately 800 million randomized affibody-encoding genes underwent magnetic- and fluorescence-activated cell sorts to isolate affibodies that bind specifically to VEGF, FGF-2, or PDGF. Monoclonal affibody-displaying yeast that exhibited binding to their protein target were isolated and sequenced. The affinities of surface-displayed affibodies for their target were estimated by incubating monoclonal yeast with 2.5-10000 nM of the target protein, followed by binding analysis using flow cytometry. Target specificity was evaluated by comparing monoclonal affibody binding between all three angiogenic proteins. Target-specific affibodies were transformed into E. coli and expressed with a hexahistidine tag and C-terminal cysteine (e.g., aa 59-65 of SEQ ID NO: 65) for purification and chemical conjugation, respectively.

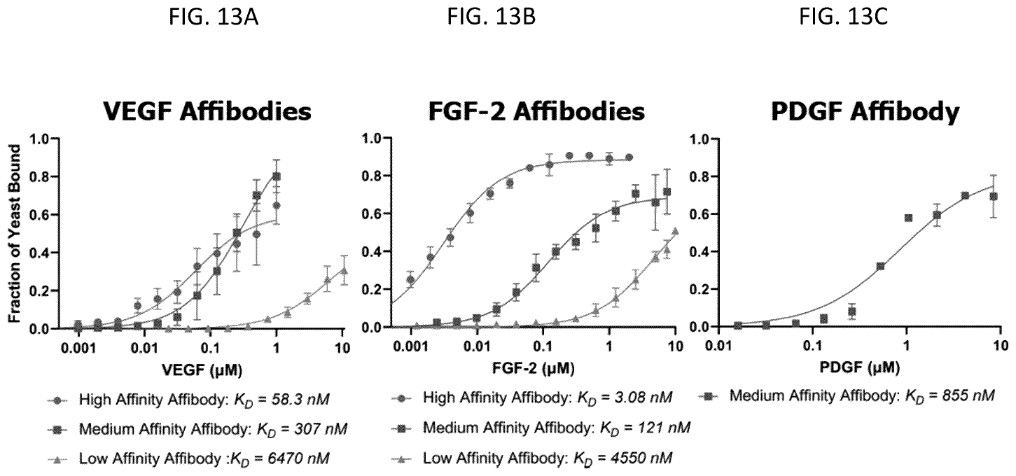

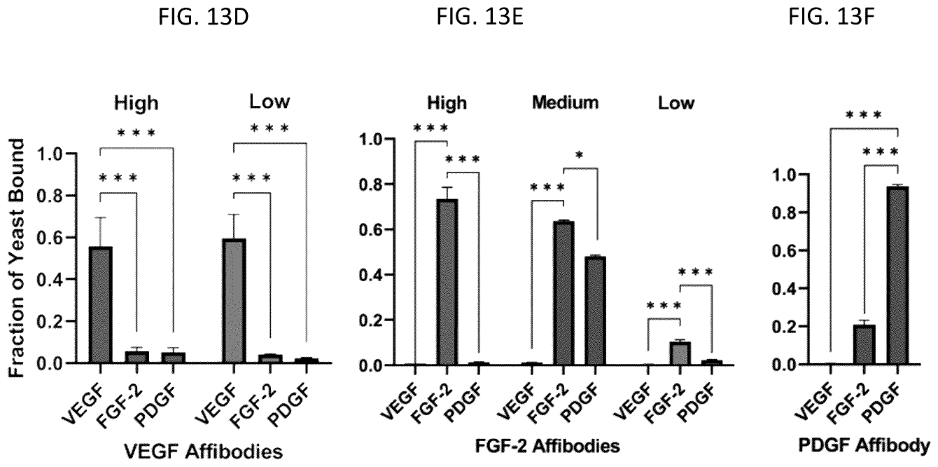

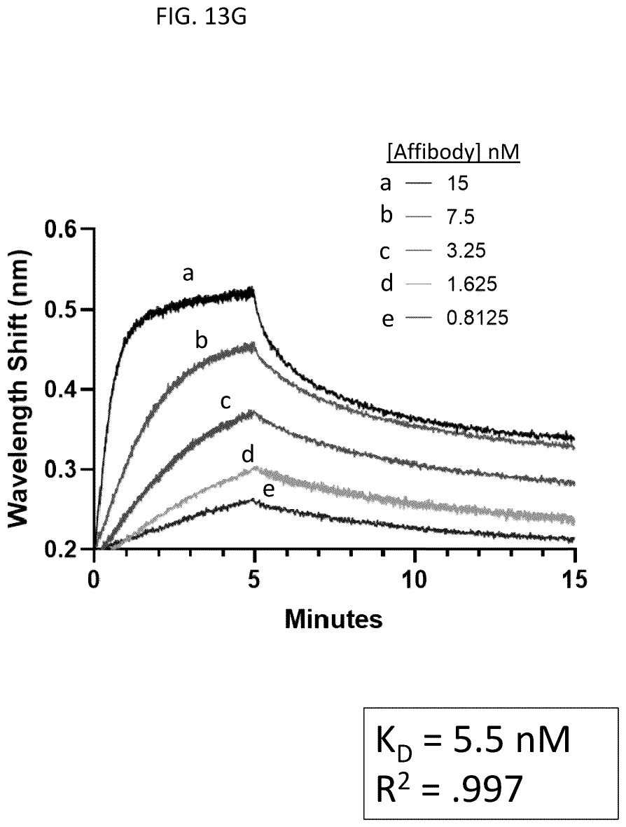

As shown in FIGS. 13A-13G, affibodies for VEGF, FGF-2 and PDGF were identified.

Affibodies with high (K_{D}=58.3±32.6 nM; SEQ ID NO: 20), medium (K_{D}=307 nM; SEQ ID NO: 21), and low (K_{D}=6470 nM; SEQ ID NO: 22) affinities for VEGF were identified; all exhibited specific binding to VEGF with negligible binding to FGF-2/PDGF. Additional VEGF affibodies were identified and are shown in SEQ ID NOs: 23-41. Such affibodies can be linked to a localization domain to form dual-affinity fusion proteins, which can be used to control the release of VEGF. For example, a medical material can be loaded with one or more dual-affinity fusion proteins bound to VEGF, and when the medical material is applied to a subject to treat a wound, and/or a bone or cartilage injury or disease (for example by applying the medical material to a wound or injury site), the dual affinity fusion proteins will control the release of VEGF.

Affibodies with high (K_{D}=3.08±0.45 nM; SEQ ID NO: 42), medium (K_{D}=121.2±36.7 nM; SEQ ID NO: 43), and low (K_{D}=4550 nM, SEQ ID NO: 44) affinities for FGF-2 were identified; the high-affinity affibody bound specifically to FGF-2, while the medium-affinity affibody exhibited binding to FGF-2 and PDGF. Additional FGF-2 affibodies were identified and are shown in SEQ ID NOs: 45-56. Such affibodies can be linked to a localization domain to form dual-affinity fusion proteins, which can be used to control the release of FGF-2. For example, a medical material can be loaded with one or more dual-affinity fusion proteins bound to FGF-2, and when the medical material is applied to a subject to treat a wound, and/or a bone or cartilage injury or disease (for example by applying the medical material to a wound or injury site), the dual affinity fusion proteins will control the release of FGF-2.

One affibody with medium (K_{D}=855 nM; SEQ ID NO: 60) affinity for PDGF was identified, which bound strongly to PDGF and weakly to FGF-2. Characterization of a high-affinity PDGF affibody is shown in FIG. 13G. Additional PDGF affibodies were identified and are shown in SEQ ID NOs: 57-59. Such affibodies can be linked to a localization domain to form dual-affinity fusion proteins, which can be used to control the release of PDGF. For example, a medical material can be loaded with one or more dual-affinity fusion proteins bound to PDGF, and when the medical material is applied to a subject to treat a wound, and/or a bone or cartilage injury or disease (for example by applying the medical material to a wound or injury site), the dual affinity fusion proteins will control the release of PDGF.

### Example 12: Identification of Affibodies for Immune Regulation

Using the methods described in Example 1, affibodies were identified for IL-4, associated with inflammatory responses.

A yeast surface display library containing approximately 800 million randomized affibody-encoding genes underwent magnetic- and fluorescence-activated cell sorts to isolate affibodies that bind specifically to IL-4. Monoclonal affibody-displaying yeast that exhibited binding to their protein target were isolated and sequenced. The affinities of surface-displayed affibodies for their target were estimated by incubating monoclonal yeast with 2.5-10000 nM of the target protein, followed by binding analysis using flow cytometry. Target specificity was evaluated by comparing monoclonal affibody binding between all three angiogenic proteins. Target-specific affibodies were transformed into E. coli and expressed with a hexahistidine tag and C-terminal cysteine for purification and chemical conjugation, respectively.

Affibodies with high (K_{D}=4 nM; SEQ ID NO: 61) and low K_{D}=92,000 nM; SEQ ID NO: 62 affinities for IL-4 were identified; all exhibited specific binding to IL-4. Additional IL-4 affibodies were identified and are shown in SEQ ID NOs: 63-64. Such affibodies can be linked to a localization domain to form dual-affinity fusion proteins, which can be used to control the release of IL-4, which manipulates the immune response such as increase or decrease the recruitment and differentiation of immune cells, to create a balanced environment for wound or injury healing. For example, a medical material can be loaded with one or more dual-affinity fusion proteins bound to IL-4, and when the medical material is applied to a subject to treat a wound, and/or a bone or cartilage injury or disease (for example by applying the medical material to a wound or injury site), the dual affinity fusion proteins will control the release of IL-4.

### Example 13: Design and Characterization of Fusion Proteins Including Collagen Binding Domain And BMP-2 Binding Affibody

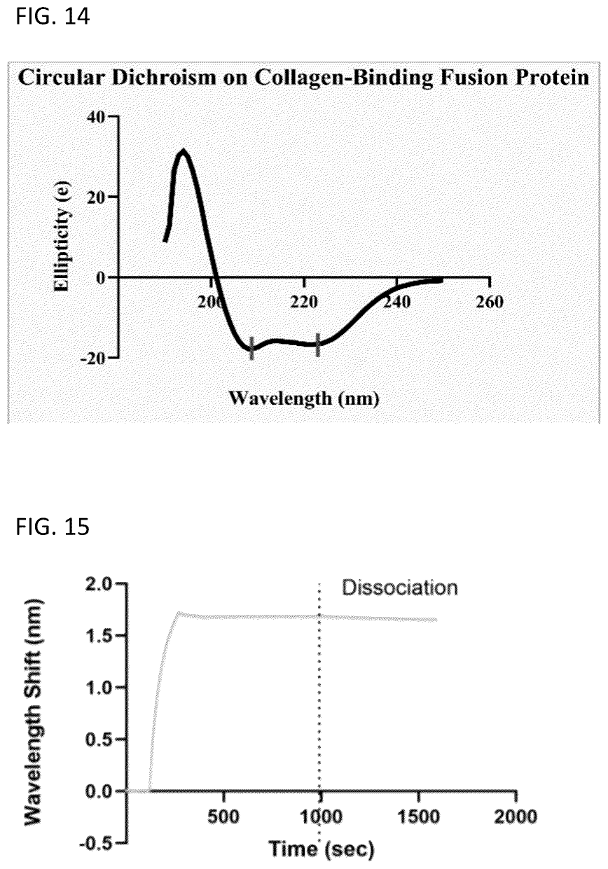

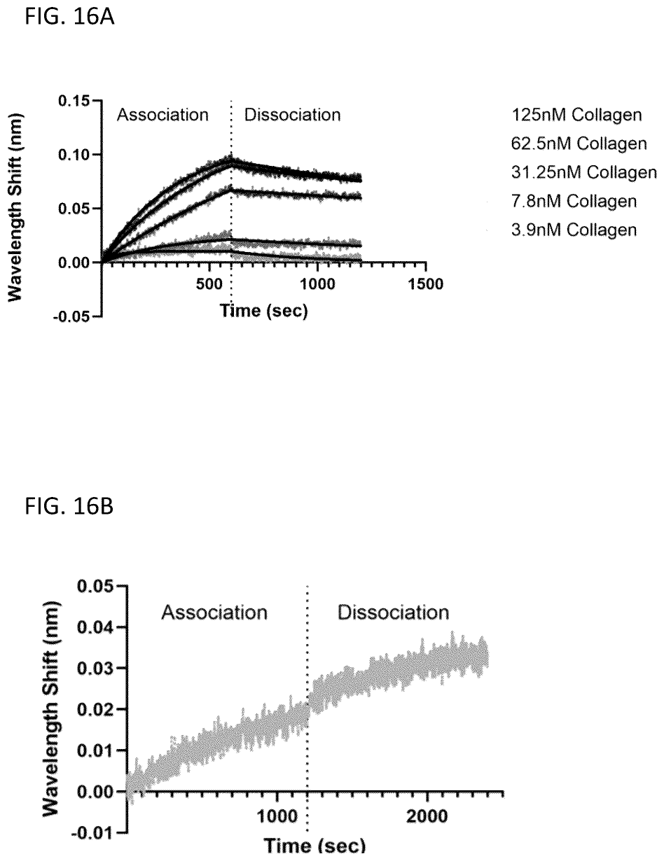

A fusion protein (SEQ ID NO: 87) including both a collagen binding domain (SEQ ID NO: 75) and a BMP-2 specific affibody domain (SEQ ID NO: 1 without the initial A) was expressed and purified. The collagen binding domain (SEQ ID NO: 75) alone was also expressed and purified. The binding affinity and specificity of the collagen binding domain was characterized by BLI techniques. The secondary structure, the binding affinity and specificity of the fusion protein was characterized using circular dichroism and BLI techniques, respectively. Using circular dichroism, it was observed that the BMP-2 specific affibody domain of the fusion protein retains the expected alpha helical secondary structure after protein expression and purification (FIG. 14).

Through BLI, a K_{D} can be obtained from specific kₒₙ and k_{off} rates, to characterize the individual strengths of different affinity interactions of the fusion protein.

Before conducting BLI characterization of the collagen binding domain portion and the entire fusion protein, it was confirmed that Type I collagen did not non-specifically bind to the Ni-NTA probe used for the BLI (FIG. 15).

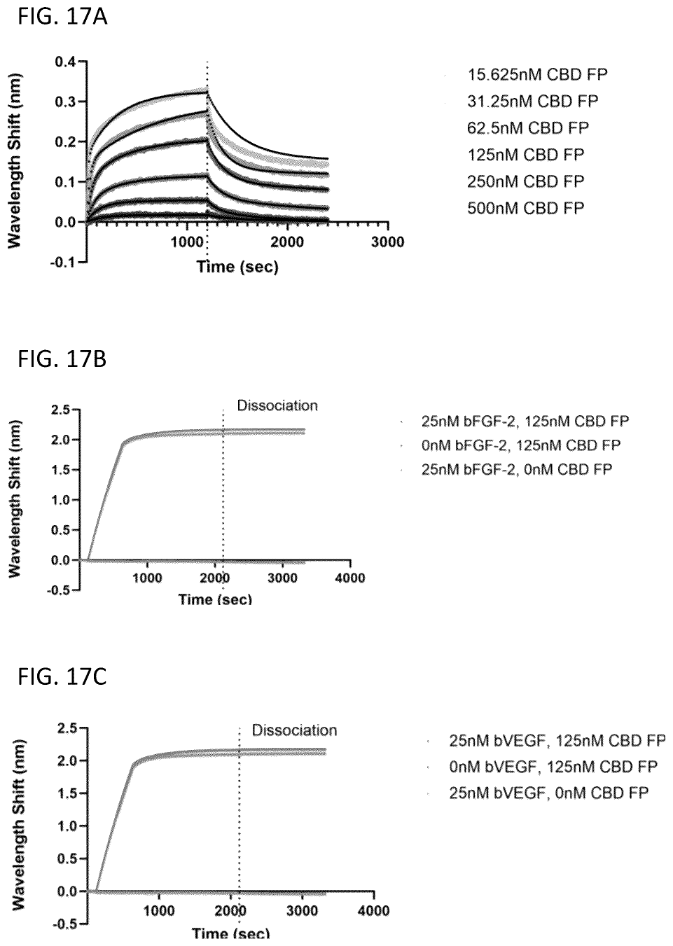

25 nM of the collagen binding domain (SEQ ID NO: 75) with a His tag (SEQ ID NO: 86) was loaded onto the Ni-NTA probe, and it was demonstrated that the collagen binding domain binds to collagen in a concentration dependent manner with a K_{D} of 16.4 nM, kₒₙ of 1.52*10^{4 1}/Ms, and k_{off} of 8.15*10^{4 1}/s, using serially diluted Type I collagen (FIG. 16A). The collagen binding domain does not bind non-specifically to BMP-2 (FIG. 16B). It was further demonstrated that the fusion protein binds to BMP-2 in a concentration dependent manner (FIG. 17A). The fusion protein does not bind non-specifically to VEGF or FGF-2, which are other growth factors commonly found in the bone healing environment (FIGS. 17B-17C).

An alternative collagen binding and BMP-2 binding fusion protein was designed (SEQ ID NO: 88), wherein the collagen binding domain (SEQ ID NO: 76) has improved affinity to collagen, and the linker sequence was adjusted to minimize the available binding surfaces of the BMP-2 affibody to collagen. The binding to collagen by the collagen binding domain is nearly pH independent and the domain itself is more stable. The ability to function independently of pH allows for more robust performance in the often-acidic environments during inflammatory healing phases. Thus, the fusion protein can function through all wound healing stages.

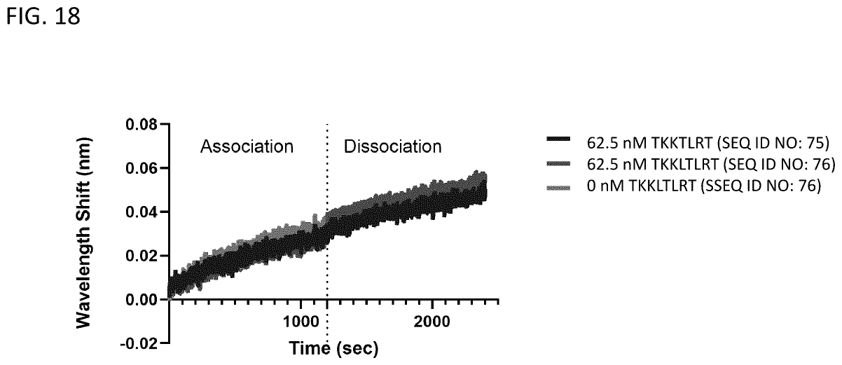

It was confirmed by BLI analysis that this collagen binding domain does not bind non-specifically to BMP-2 (FIG. 18).

Using AlphaFold and ZDOCK, it was determined that a rigid helical linker (SEQ ID NO: 83) was the most effective among all tested linkers (SEQ ID NOs: 80-81 and 83-85) in separating the BMP-2 specific affibody domain from the collagen binding domain (FIG. 19) and thus most effective in preventing the BMP-2 affibody from occluding collagen binding.

Fusion proteins (SEQ ID NOs: 87-91) including both a collagen binding domain and BMP-2 specific affibody with a His-tag (SEQ ID NO: 86) at their C-termini can be expressed and purified. Their secondary structure and binding affinity and specificity can be characterized using circular dichroism and BLI techniques, respectively, using the methods provided herein.

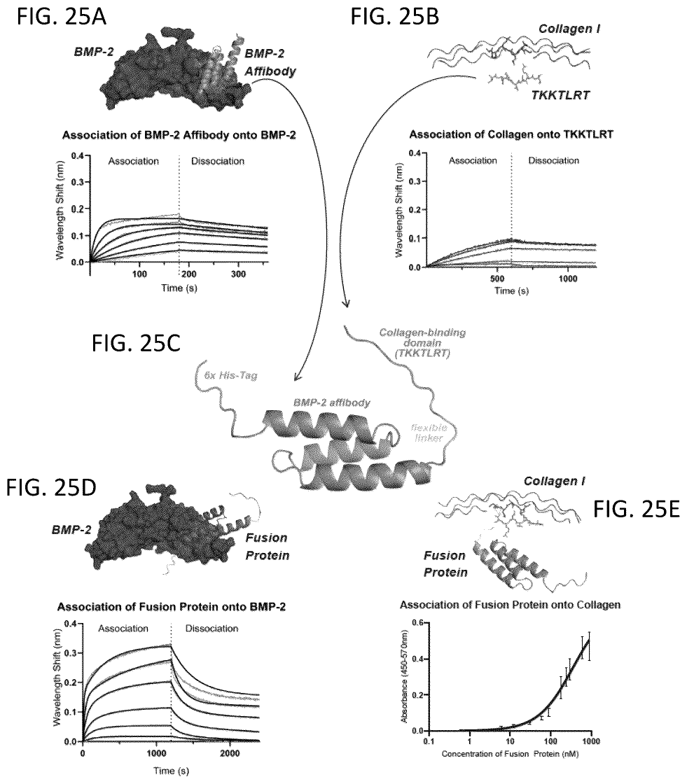

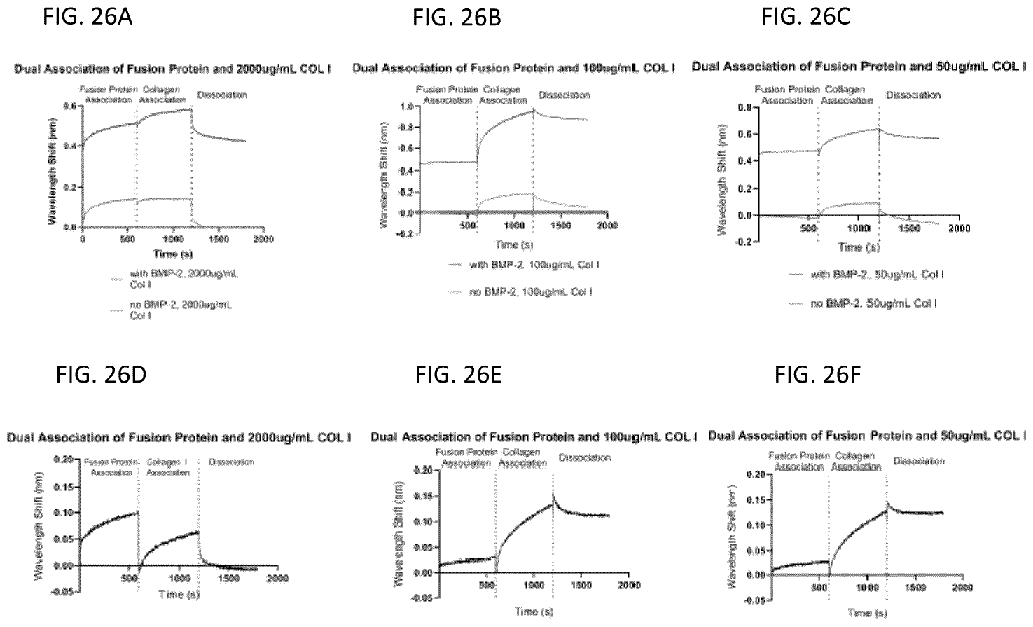

A high affinity BMP-2 specific affibody (SEQ ID NO: 1) binds to BMP-2 with a K_{D} of 10.7 nM (FIG. 25A). A collagen binding domain TKKTLRT (SEQ ID NO: 75) binds to type I collagen with a K_{D} of 16.4 nM (FIG. 25B). A dual-affinity fusion protein (SEQ ID NO: 87) combining these domains with a flexible glycine-serine linker (SEQ ID NO: 80) and with a 6×His-tag added to the C-terminus was constructed and purified (FIG. 25C). The fusion protein binds to BMP-2 with a K_{D} of 66.8 nM (FIG. 25D). To quantify the affinity of the fusion protein to collagen, an enzyme-linked immunosorbent assay (ELISA) was performed with an anti-6×His-tag antibody, and the K_{D} was determined to be 363 nM (FIG. 25E). BLI was then used to confirm that the fusion protein binds both to BMP-2 and to collagen. Binding curves of the fusion protein binding to BMP-2 loaded probes (or empty probes), and binding of 2000 μg/mL, 100 μg/mL, or 50 μg/mL collagen to the bound fusion protein are shown in FIGS. 26A-26C. After removing the background and non-specific binding signals, two separate binding interactions can be clearly observed (FIGS. 26D-26F). These results demonstrate that the fusion protein maintains two separate binding interactions towards BMP-2 and collagen. Because collagen is far more abundant in the environment where the fusion protein is used than BMP-2, the fusion protein's slightly lower affinity for collagen, compared to its affinity for BMP-2, enables it to remain anchored to collagen while releasing BMP-2.

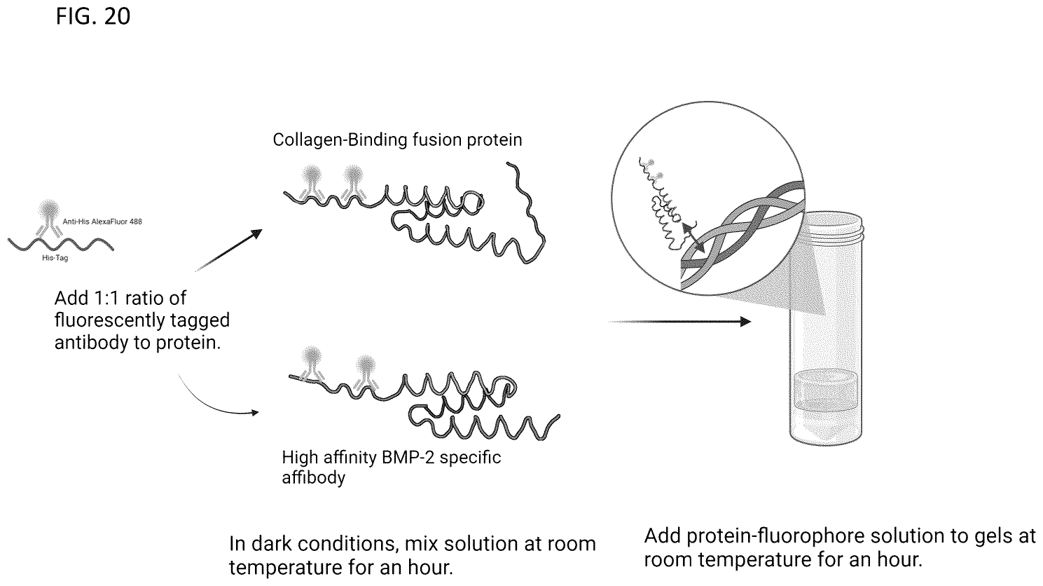

To demonstrate that the fusion protein binds to collagen hydrogels and/or sponges, a fluorescence retention assay can be performed. An anti-His-tag antibody with AlexaFluor 488 fluorophore or AlexaFluor 488 NHS ester can be used to fluorescently label the fusion protein. Collagen hydrogels and/or sponges can then be incubated with the fluorophore-labeled fusion protein over 1-24 hours to promote binding of the fusion protein (FIG. 20). Then the material (collagen hydrogel or sponge) is placed in buffer (saline, cell culture medium, etc.) and incubated for 1-7 days to measure retention of the fusion protein on the material surface. A plate reader is used to measure fluorescence of the supernatant and/or material over multiple time points. The fluorescence of the material will decrease, and the fluorescence of the supernatant will increase, as the fusion protein detaches from the material and goes into solution. This will allow the determination of the binding of the fusion protein to the material. A control experiment can also be conducted where the anti-His-tag antibody with AlexaFluor 488 fluorophore is used to label a BMP-2 affibody, and the BMP-2 affibody is loaded into the same material. It is expected that the BMP-2 affibody will not be retained on the material because it does not contain a collagen-binding domain.

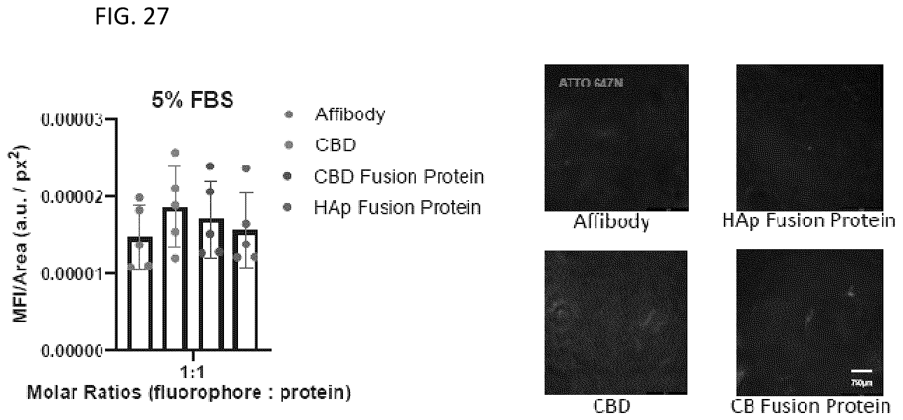

Using 5% FBS blocking buffer, 6 mm collagen sponges were exposed to the same amount of affibody (SEQ ID NO: 1), collagen-binding domain (SEQ ID NO: 75), collagen-binding fusion protein (SEQ ID NO: 87), and HA-binding fusion protein (SEQ ID NO: 108), and mean fluorescence intensity was measured from images taken by a confocal microscope following a 24-hour incubation (FIG. 27). The collagen-binding domain and collagen-binding fusion protein demonstrated the highest retention in the collagen sponge after 24 hours.

Further, using collagen sponges and/or hydrogels, a controlled release assay can be conducted where fusion protein and BMP-2 is loaded onto the material, and the amount of BMP-2 released from the collagen-bound fusion protein is measured by ELISA over multiple timepoints, for example over 1-4 weeks. The amounts of BMP-2 and fusion protein can be varied, such that the effects of different ratios of fusion protein to BMP-2 on overall BMP-2 release from the materials are investigated. This will allow the determination of the optimal amount of the fusion protein to be loaded onto or carried by a medical material (such as a collagen hydrogel or sponge) to ensure controlled BMP-2 release from the medical material.

To measure fusion protein retention in collagen sponge using ELISA, the following method can be used.

Cut 6 mm diameter, 1 mm width sponges and place them in 2 mL low retention tubes. Prepare 1× molar excess of fusion protein to collagen sponge and add to the sponges. Add 200 μL of 0.1% PBSA and centrifuge tubes at 4° C. overnight. Collect 200 μL of liquid, and add 1000 μL of fresh 0.1% PBSA. Collect 200 μL of liquid, and replace with fresh 0.1% PBSA after 1, 3, 5, and 7 days. Using the ELISA procedure described below, measure the concentration of fusion protein released from collagen sponges.

Ladder preparation: 2500 ng/mL, 2000 ng/mL, 1500 ng/mL, 750 ng/mL, 500 ng/mL, 250 ng/mL, 100 ng/mL, and 0 ng/mL of fusion protein.

1) Add 100 μL/well of 80 μL collagen (Enzo Life Sciences COL I Rat Tail #)+3920 μL water to a plate, incubate overnight at RT.

2) The next day, wash with PBST, and add 300 μL/well of 1× Reagent Diluent (P352891); incubate for 2 hours at RT. Wash 1× in PBST.

3) Add 100 μL/well of sample; incubate for 2 hours at room temperature. Wash 3×.

4) Add 100 μL/well of 0.8 μL of detection Ab (biotinylated Anti-His) with 4.8 mL of Reagent Diluent; incubate for 2 hours at room temperature. Wash 1×

5) Add 100 μL/well of 50 μL of strep-HRP(P416477) in 4950 μL of Reagent Diluent; incubate for 20 minutes. Wash 1×.

6) Add 100 μL/well of TMB substrate (TMSKY04-6) at room temperature; incubate for 20 to 30 minutes until ladder fully develops.

7) Add 50 μL/well of stop solution before reading the plate at 450 nm and 540 nm absorbance wavelengths.

Take sponges from 7-day retention. Cryosection sponges and place on glass slides with sections of 20 m thickness. Wash slides in 100%, 75%, 50%, 25% and ddH₂O for 1 minute each before treating the sponges with blocking buffer for 2 hours at room temperature. Add primary antibody (biotinylated anti-his) for one hour before washing three times with PBST. Next, incubate sections for two hours with secondary antibody (streptavidin-AF647, 1:200) for 2 hours at room temperature before washing three times with PBST and then once with water. Mount sections with Permount and let them cure overnight.

Using Leica Thunder confocal fluorescence microscope, image sectioned and stained slides at 5× magnification. Analyze images in ImageJ for mean fluorescence intensity and plot for each tested group.

To measure BMP-2 release from fusion proteins bound to collagen sponge, the following method can be used.

Place a collagen sponge (˜6 mm diameter, ˜1 mm width) in a 2 mL low retention tube. Add fusion protein (e.g., 500× molar excess to BMP-2) and 200 μL of 0.1% PBSA to the tube. Centrifuge the tube for 1 hour at room temperature, then centrifuge the tube overnight at 4° C. Wash the sponge once with 1500 μL 1% PBSA to remove excess unbound fusion protein. Add BMP-2 (e.g. 100 μL of 1 ng/μL BMP-2 in 0.1% PBSA) to the tube, and centrifuge the tube at 4° C. overnight. The next day, add 100 μL of 0.1% PBSA to the tube, and collect 200 μL of supernatant for encapsulation analysis. Immediately add 1 mL of 10% FBS to the tube, then collect 200 μL of supernatant as Day 0 sample, followed by addition of 200 μL of 10% FBS. Repeat these steps to collect samples at Day 1, 3, 5, 7, 10, and 14. Analyze data (e.g., in GraphPad) and plot release curve using a nonlinear curve fitting equation. The concentration of fusion protein can be varied (e.g., 100×, 500×, 1000×, etc. molar excess of BMP-2) while keeping the concentration of BMP-2 constant (e.g., at 1 ng/μL). The concentration of BMP-2 can be varied (e.g., at 0.5, 1, 3, 5, 10, etc. ng/μL) while keeping the concentration of fusion protein constant.

In vivo BMP-2 localization can be assessed by subcutaneously implanting collagen sponges loaded with fluorescently labeled BMP-2 into the backs of rats (4 implants per rat) and performing in vivo imaging using an in vivo imaging system (IVIS). Fusion protein and BMP-2 tagged with a near infrared Vivotag 750 NHS ester will be loaded into collagen sponges for surgical implantation. The IVIS measures the retention of BMP-2 within the implant sites at 0, 1, 2, 4-, 7-, 10-, and 14-days post-surgery. In vivo mineralization of excised subcutaneous implants can be measured using x-ray radiography, micro-computed tomography after 4 weeks of implantation.

In a sterile surgical environment, after installing a polysulfone fixation plates onto femurs of equal numbers of male and female 13-week-old Sprague Dawley rats, 6 mm bilateral bone defects can be made to femurs using a bone saw. After creating the defects, 6 mm long treatment collagen sponges from Integra Life Sciences can be implanted. As negative controls, sponges with no protein and with BMP-2 specific affibody only can be implanted. As a positive control, sponges only containing BMP-2 can be implanted. Our treatment sponge can contain BMP-2 bound fusion protein. Using a similar analysis workflow as described above, x-ray and micro-CT can be performed on live rats weeks 2, 4, 8, and 12 to assess the amount of mineralization surrounding the fixation plate and within the defect area. Micro-CT can be performed on rats from weeks 2, 8 and 12.

Immunohistochemistry can be performed on excised tissue at the end of 12 weeks using H&E staining, Safranin O, Fast Green, and Picrosirius red. H&E staining is expected to reveal improved bone quality from a greater number of bone cell nuclei on scaffolds containing both fusion protein and BMP-2, compared to the scaffolds that contained BMP-2 and no fusion protein. Safranin O will reveal cartilage formation, while Picrosirius will reveal highly organized collagen and lamellar bone formation.

The above methods can be repeated for fusion proteins specific for any other therapeutic proteins, including VEGF, FGF-2, PDGF, GM-CSF, and IL-4, or any combinations thereof, such as a fusion protein specific for GM-CSF and a fusion protein specific for IL-4.

### Example 14: Design and Characterization of Fusion Proteins Including Hydroxyapatite Binding Domain And BMP-2 Binding Affibody

Fusion proteins (SEQ ID NOs: 108-112 and 126) including both a hydroxyapatite (HA) binding domain (any of SEQ ID NOs: 77-79 and 127) and a BMP-2 specific affibody (SEQ ID NO: 1) and modified with a His-tag (SEQ ID NO: 86) at their C-termini were expressed, purified and characterized.

DNA Cloning and Confirmation of Protein Expression: DNA sequences for each of the unique fusion proteins modified with a methionine (start codon) at the N-terminal and a 6-His-tag at the C-terminal were obtained. Using a double digest restriction enzyme protocol with NcoI and XhoI cut sites, the DNA insert was cloned into a pET28b+ vector. The pET28b+ vector confers kanamycin resistance and uses an isopropyl β-D-1-thiogalactopyranoside (IPTG) inducible T7 promoter for protein expression. Vectors were transformed into BL21 (DE3) chemically competent E. coli (New England BioLabs) per the manufacturer's protocols. 100 μL of transformed E. coli were plated on kanamycin selective growth plates (10 g yeast extract, 20 g bacto peptone, 20 g dextrose, 16 g bacto agar, 50 mg kanamycin sulfate, 1 L distilled water) and incubated at 37° C. for 24 h. Colonies were selected and expanded in 20 mL Luria-Bertani (LB) broth (Thermo Fisher) supplemented with 20 μL of 50 mg/mL of kanamycin sulfate in water until an optical density at 600 nm (OD600) of 0.8 was reached. 4 mL of the culture were lysed and used to obtain plasmid DNA for sequence confirmation (Plasmid Miniprep Kit; Zymo Research) via whole-plasmid sequencing (Plasmidsaurus). A new overnight culture was prepared for each colony that was confirmed to contain the desired fusion protein sequence. These cultures were induced to produce protein with 0.5 M IPTG after reaching an OD600 of 0.8 and incubated further for 4 hours at 37° C. The induced and uninduced (control) E. coli were lysed using Bug Buster Protein Extraction Agent (Millipore Sigma) and centrifuged to separate the soluble proteins and the lysate. The soluble proteins were prepared for SDS-PAGE by diluting 18 μL of sample in 6 μL Laemmli buffer (BioRad) supplemented with 10 v/v % β-mercaptoethanol (BioRad) and heated for 5 minutes at 90° C. The samples were loaded into a 4-20% Mini-PROTEAN® TGX™ Precast Protein Gel (BioRad), run under denaturing conditions at 200 V for 35 minutes, stained with Coomassie blue dye, and imaged on an Azure 200 Gel Imager.

Large Scale Protein Expression and Urea Dialysis Protocol for Protein Purification: Due to the structural and electrochemical properties of the fusion proteins, most of the fusion proteins were trapped in inclusion bodies of the pelleted growth culture. Thus, a new protein purification protocol was developed using urea dialysis to denature and extract the fusion protein from the inclusion bodies and slowly renature them via successive dilutions in PBS.

Transformed E. coli were grown in 20 mL LB broth supplemented with 0.5 mM kanamycin and incubated overnight at 37° C. The E. coli were then transferred into 1.8 L of Terrific Broth (TB) supplemented with 0.5 mM kanamycin and 500 μL of anti-foam 204 (Thermo Scientific) and cultured at 37° C. in a LEX-10 bioreactor (Epiphyte3). When OD600 reached approximately 1.4, 1.8 mL of 0.5 M IPTG was added to the growth vessel to obtain a final IPTG concentration of 0.5 μM, and the temperature was reduced to 18° C. for 18 hours for induction of protein expression. After 18 hours, the culture was centrifuged for 20 minutes at 4° C. at 6000 RPM, and the cell pellet was removed and transferred to two 50 mL conical tubes. The collected cell pellet was resuspended completely in 10 mL of pH 8 buffer (100 mM monobasic sodium phosphate, 10 mM Tris, and 8 M Urea). The lysate was incubated at room temperature for 30 minutes. After 30 minutes, the lysate was centrifuged at 10000 g for 30 minutes at room temperature. The supernatant was collected and combined with 1 mL of Cobalt-NTA Agarose Beads (GoldBio; Cobalt beads). The mixture was rotated for 1 hour at room temperature. After an hour, the binding suspension was transferred to a Econo-Column® chromatograph column (Biorad) with a capped bottom outlet. The bottom cap of the column was removed to collect the flow-through. The column was washed with 5 mL of pH 6.3 buffer (100 mM monobasic sodium phosphate, 10 mM Tris, and 8 M Urea). The fusion protein was finally eluted using 5×0.5 mL of pH 4.5 buffer (100 mM monobasic sodium phosphate, 10 mM Tris, and 8 M Urea). Each eluate fraction was collected in a separate tube, and the protein concentration of each fraction was measured using an IMPLEN spectrophotometer. Following protein collection, the fusion proteins underwent stepwise dialysis to buffer exchange the protein from 8 M urea into PBS. The eluted protein was added to dialysis tubing (Molecular weight cutoff: 6-8 kDa, VWR) and transferred to a 1 L solution of 8 M urea. The protein was dialyzed for one hour before replacing 500 mL of the volume with 500 mL of 4 M urea solution. After 1 hour had passed, another half of the volume was replaced with another 500 mL of 4 M Urea. The same solution replacement occurred for 2 M Urea, 1 M Urea, and 1 M PBS solution until the protein was dialyzed in 1 M PBS solution for 1 hour. The total time for dialysis was approximately 7-8 hours. The final protein was transferred out of the dialysis tubing into centrifuge tubes, and the final protein concentration was measured using UV-vis spectrophotometry. The newly soluble proteins were prepared for SDS-PAGE by diluting 18 μL of sample in 6 μL Laemmli buffer (BioRad) supplemented with 10 v/v % β-mercaptoethanol (BioRad) and heated for 5 minutes at 90° C. The samples were loaded into a 4-20% protein gel, run under denaturing conditions at 200 V for 35 minutes, stained with Coomassie Blue dye, and imaged on an Azure 200 Gel Imager.

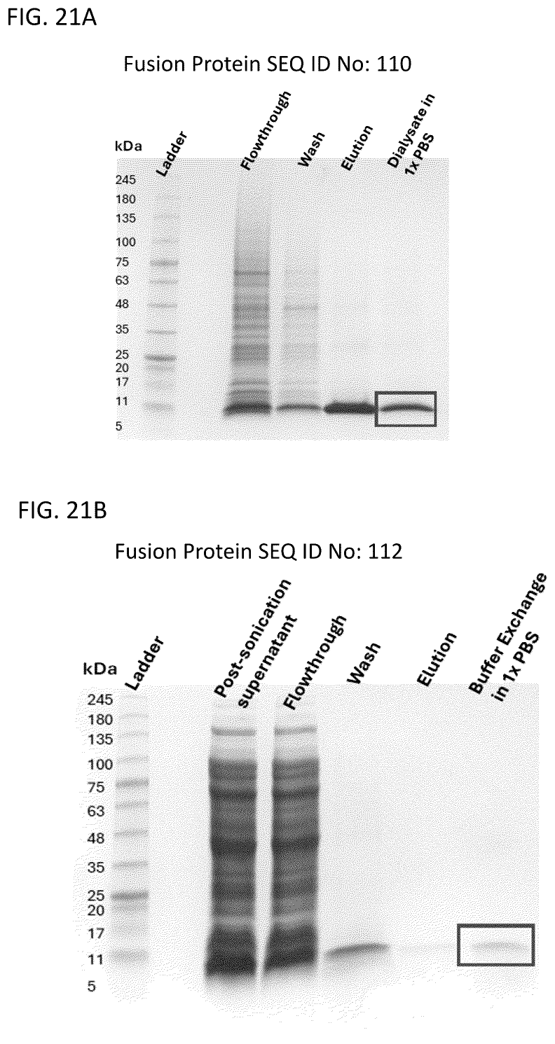

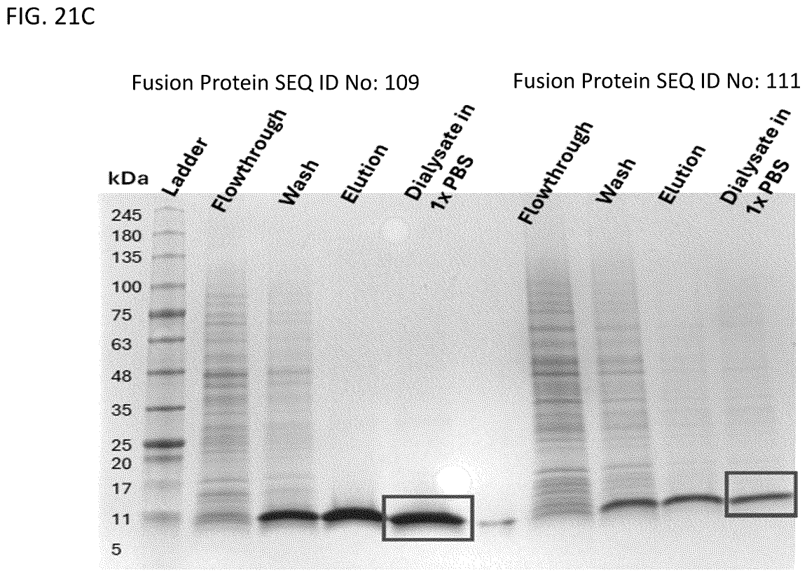

Fusion Protein Size and Purity: FIGS. 21A-21C are gel electrophoresis images of multiple protein fractions obtained during the purification process of fusion proteins (SEQ IDs 109-112, plus the His-tag), including initial flowthrough, wash, and elution fractions from the chromatography column, and the final dialyzed product. All fusion proteins were successfully purified using this new method and appeared at the expected molecular weight of 8-10 kDa. The molecular weights of the fusion proteins were predicted based on their amino acid sequences using EXPASY ProtParam to be 8631.51 g/mol (SEQ ID NO: 110), 8333.24 g/mol (SEQ ID NO: 112), 9259.24 g/mol (SEQ ID NO: 109), and 8960.98 g/mol (SEQ ID NO: 111).

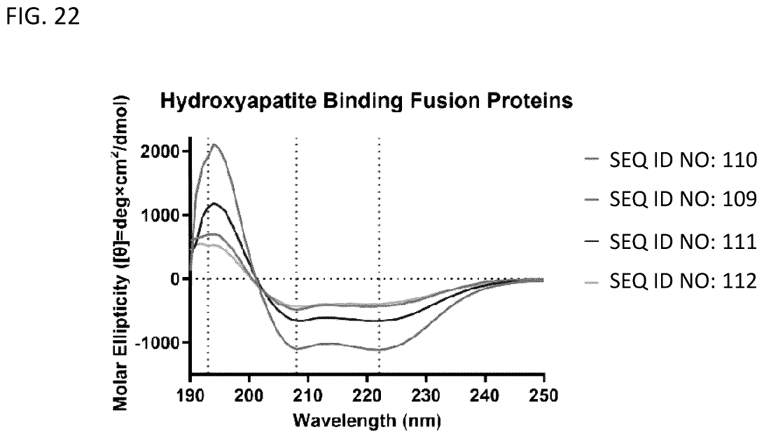

Characterization of Fusion Protein Secondary Structure: A Jasco J-815 circular dichroism spectropolarimeter (CD Spec; JASCO) was used to characterize the secondary protein structure of the fusion proteins. Purified fusion proteins were buffer-exchanged into PBS pH 6.9 using Zeba columns and diluted to a concentration below 50 μM and in 10 mM of Tris at pH 7.4. The fusion protein was loaded into a quartz cuvette with 1 mm path length and placed in the CD Spec. The CD value and the high-tension voltage were collected over a wavelength range of 190-250 nm. The CD values were converted to molar ellipticity using the molecular weight and concentration of each fusion protein (FIG. 22). Since the BMP-2 affibody, which is the larger domain of the fusion protein, is alpha helical, it was expected that fusion proteins would demonstrate alpha-helical secondary structures. This was confirmed using CD, as all fusion proteins produced CD spectra with troughs at 208 nm and 222 nm and a peak at 190 nm, which is indicative of an alpha helical secondary structure. Furthermore, the fusion proteins containing the rigid linkers, which are also alpha helical, show higher alpha helical contents.

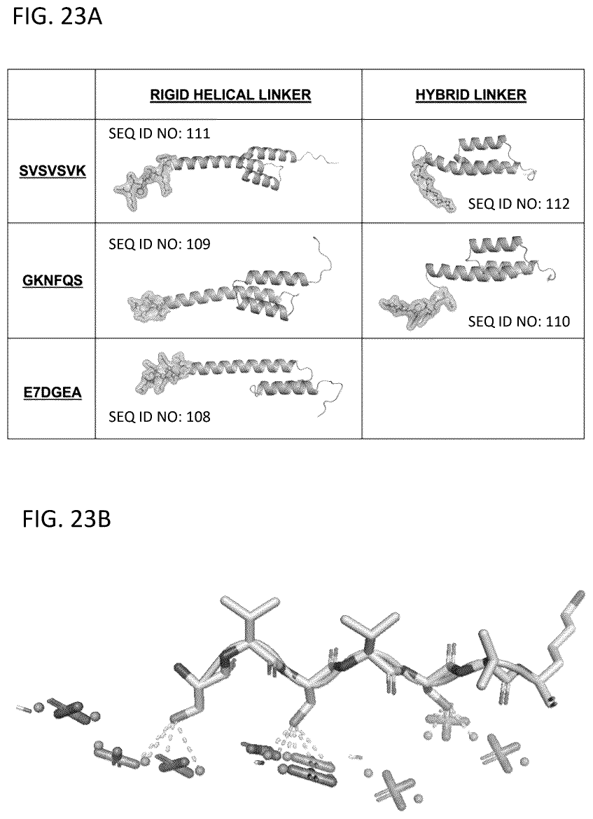

Secondary structures of the fusion proteins predicted by AlphaFold3 are shown in FIG. 23A. The predictive structure output from AlphaFold3 was then used to examine the availability of electrostatic binding interactions between the HA binding domain interface and HA crystal. PyMOL was used to model the binding between the SVSVSVK binding domain (SEQ ID NO: 79) (tan) and HA (FIG. 23B), demonstrating the regularly interspersed interactions (dotted yellow lines) between the hydroxyl groups (red) of the serine side chains of the binding domain to the positively charged calcium ions (green) of hydroxyapatite.

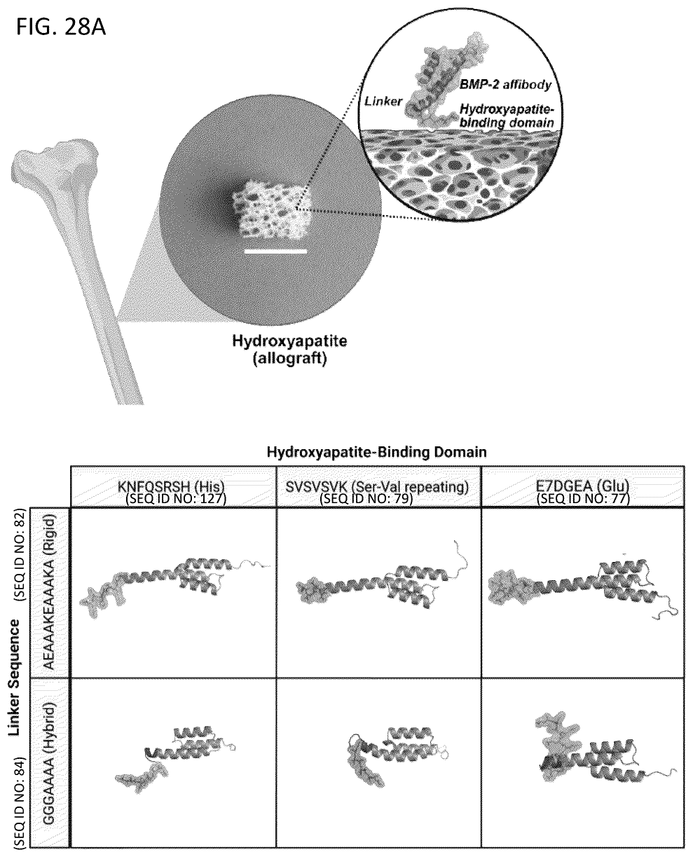

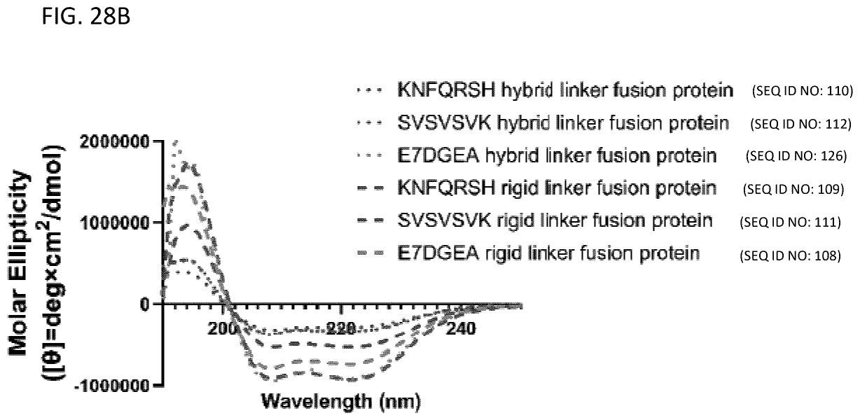

Six fusion proteins (SEQ ID NO: 110, 112, 126, 109, 111, or 108 plus a C-terminal 6×His-tag) were constructed with the linker (SEQ ID NO: 82 or 84) and HA-binding domain (SEQ ID NO: 127, 79, or 77) shown in FIG. 28A, and with a BMP-2 specific affibody (SEQ ID NO: 1 without the initial A). The secondary structure of the fusion proteins were characterized by CD (FIG. 28B). The fusion proteins are primarily alpha helical due to the structure of the affibody. Additionally, since the rigid linker (SEQ ID NO: 82) and the E7DGEA binding domain (SEQ ID NO: 77) also form a helical structure, the fusion proteins with either of these domains exhibit more prominent troughs at 208 nm and 222 nm.

Their binding affinity and specificity can be characterized by BLI techniques, using the methods provided herein.

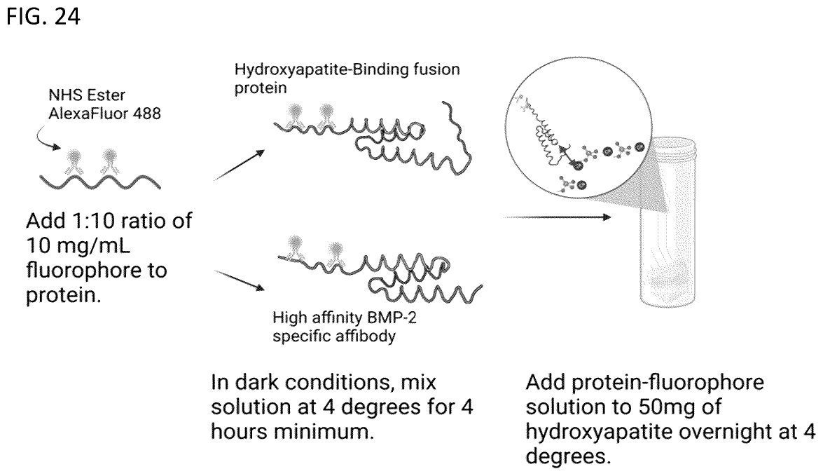

To demonstrate that the fusion protein binds to HA and/or bone allografts, a fluorescence retention assay can be performed. An anti-His tag antibody with a fluorophore (e.g., AlexaFluor 488 fluorophore), or an amine-reactive fluorescent tag (e.g., AlexaFluor 488 NHS ester) can be used to fluorescently label the fusion protein. Allograft bone chips or HA powder or pellets can then be incubated with the fluorophore-labeled fusion protein over 1-24 hours in 1M NaHCO₃ to promote binding of the fusion protein. Then the material (allograft bone chips or HA powder or pellets) is placed in an appropriate physiological buffer (saline, serum, cell culture medium, etc.) and incubated for 1-7 days to measure retention of the fusion protein on the material surface. A plate reader can be used to measure fluorescence of the supernatant and/or material over multiple time points based on the excitation and emission wavelengths of, e.g., AlexaFluor 488 and FITC fluorophores (FIG. 24). The fluorescence of the material will decrease, and the fluorescence of the supernatant will increase, as the fusion protein detaches from the material and goes into solution. This will allow the determination of the binding of the fusion protein to the material. A control experiment can also be conducted where the anti-His tag antibody with AlexaFluor 488 fluorophore is used to label a BMP-2 affibody, and the BMP-2 affibody is loaded onto the same material. It is expected that the BMP-2 affibody will not be retained on the material because it does not contain a hydroxyapatite-binding domain.

Additionally, radiant efficiency of the fluorescent proteins in the supernatant or on the materials can also be measured using an in vivo imaging system (IVIS). Further, the amount of fusion protein released from the HA powder or pellets after three extensive wash steps of 1500 μL of 1×PBS can be detected using a micro bicinchoninic acid (BCA) colorimetric assay through absorbance reading at 562 nm wavelength over 30 minute, 1 hour, 3 hour, and 5 hour timepoints on a plate reader and compared to a bovine serum albumin standard curve.

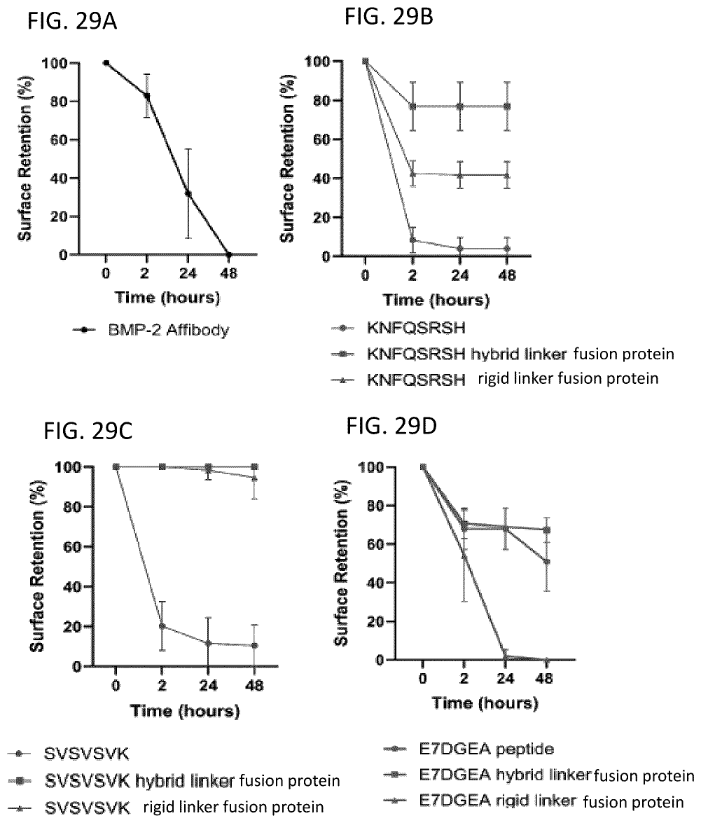

Micro BCA was used to quantify fusion protein retention to HA pellets over 48 hours. For the HA-binding domain of SEQ ID NO: 127 or 79, the binding domain alone retains poorly to the HA pellets, whereas the fusion proteins incorporating these domains (SEQ ID NOs: 110, 112, 109, and 111) show significantly improved retention (FIGS. 29B-29C). For the HA-binding domain of SEQ ID NO: 77, the fusion protein incorporating the binding domain with a hybrid linker retained better than the binding domain alone (FIG. 29D). BMP-2 affibody does not show any retention (FIG. 29A). The SVSVSVK (SEQ ID NO: 79) containing fusion proteins demonstrated the highest level of retention over 48 hours, compared to the other fusion proteins tested (FIG. 29C).

Furthermore, using the bone chips or HA powder or pellets, a controlled release assay can be conducted where fusion protein and BMP-2 is loaded onto the material, and the amount of BMP-2 released from the allograft-bound fusion protein is measured by ELISA over multiple timepoints, for example over 1-4 weeks. The amounts of BMP-2 and fusion protein can be varied, such that the effects of different ratios of fusion protein to BMP-2 on overall BMP-2 release from the materials are investigated. This will allow the determination of the optimal amount of the fusion protein to be loaded onto or carried by a medical material (such as a bone allograft) to ensure controlled BMP-2 release from the medical material.

Cumulative BMP-2 release was measured for different fusion proteins bound to HA pellets. 25 mg of HA granules were placed into 2 mL tubes. 500× molar excess of the following proteins to BMP-2 were prepared: BMP-2 affibody (SEQ ID NO: 1), fusion protein SEQ ID NO: 110, fusion protein SEQ ID NO: 112, fusion protein SEQ ID NO: 126, fusion protein SEQ ID NO: 109, fusion protein SEQ ID NO: 111, and fusion protein SEQ ID NO: 108. The affibody or fusion protein was added to the pellets in 200 μL PBS. The tubes were then spun at RT for 1 hour, and spun overnight at 4° C. The pellets were washed once with 1500 μL 1% PBSA to remove excess unbound fusion protein. 100 μL of 1 ng/μL stock of BMP-2 in 0.1% PBSA was added to each of the tubes, which were then spun at 4° C. overnight. The next day, 100 μL of 0.1% PBSA was added to each tube, and 200 μL of supernatant was collected for encapsulation analysis. 1 mL of 10% FBS was immediately added to each tube. 200 μL of supernatant was then collected as the Day 0 sample, followed by addition of 200 μL of 10% FBS. These steps were repeated to collect samples at Day 1, 3, 5, 7, 10, and 14. The above process will also be used for measuring BMP-2 release for different fusion proteins bound to bone dots.

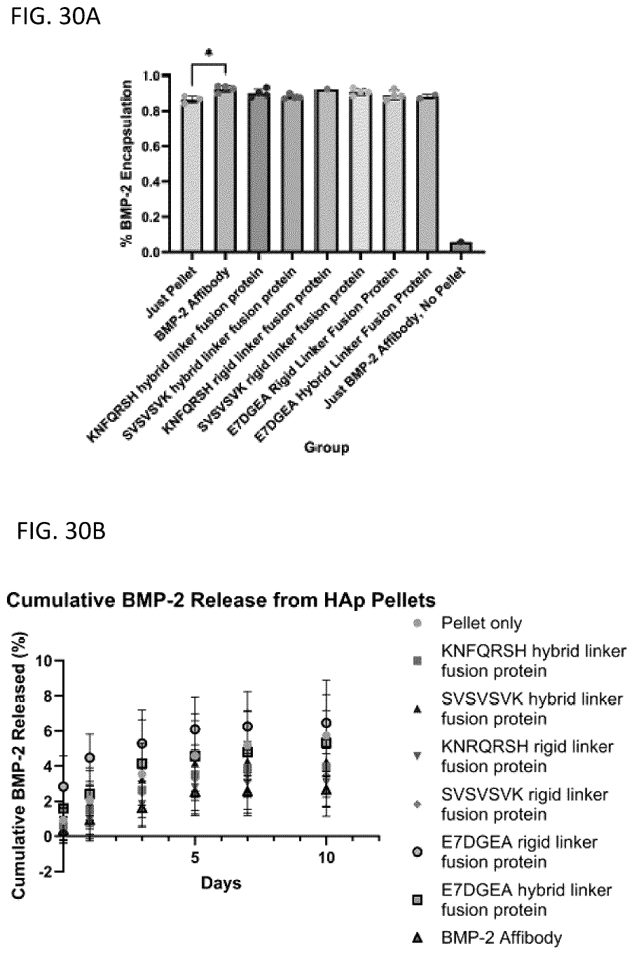

For initial BMP-2 encapsulation, there was no significant difference between all groups, except for the pellet only and affibody groups (FIG. 30A). The fusion proteins containing the E7DGEA binding domain (SEQ ID NO: 77) showed greater burst release of BMP-2, compared to pellet alone (FIG. 30B). Affibody alone released the least amount of BMP-2 (FIG. 30B). The other fusion proteins showed release profiles that fall between the pellet only group and affibody only group (FIG. 30B).

Cumulative BMP-2 release was also measured for different fusion proteins bound to allograft. Approximately equal masses of allografts were placed into separate tubes for the same groups described above. 500 μL of 5% PBSA was added to each tube of allograft for blocking, and the tube was then spun at RT for 1 hour, followed by removal of the liquid. 200 μL of 5% PBSA, and 500× molar excess of the affibody or fusion protein to 100 ng of BMP-2 were added to each tube. The tubes were spun at 4° C. overnight. The next day, all liquid was removed, and the allograft was washed once in 1500 μL of 1% PBSA. 100 ng of BMP-2 in 1% PBSA at 1 ng/μL was added. The tubes were then spun at 4° C. overnight. The next day, 100 μL of 1% PBSA was added to each tube, 200 μL of supernatant was collected for encapsulation analysis. 1 mL of 10% FBS was added to each tube. 200 μL of supernatant was then collected as the Day 0 sample, followed by addition of 200 μL of 10% FBS. These steps were repeated to collect samples at Day 1, 3, 5, 7, 10, and 14.

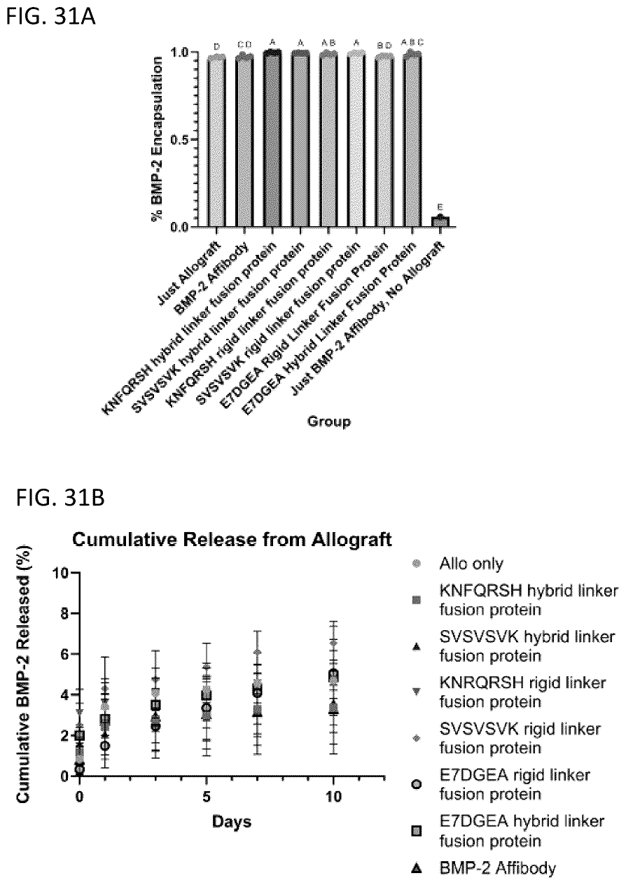

Initial BMP-2 encapsulation data indicate more instances of significant differences between groups, notably between other groups and just allograft or affibody (FIG. 31A). Over a 10-day period, the fusion proteins containing the hybrid linker (SEQ ID NO: 84) demonstrated release profiles that most closely resemble that of the affibody only (FIG. 31B). This indicate a role of the linker sequence for fusion protein function on allograft surface.

Scanning electron microscopy, x-ray photoelectron spectroscopy, and other surface characterization methods can be used to evaluate the fusion proteins on the bone allograft surface.

In vivo BMP-2 localization can be assessed by subcutaneously implanting allograft loaded with fluorescently labeled BMP-2 into the backs of rats (4 implants per rat) and performing in vivo imaging using an in vivo imaging system (IVIS). Fusion protein and BMP-2 tagged with a near infrared Vivotag 750 NHS ester can be loaded into allograft for surgical implantation. IVIS can measure the retention of BMP-2 within the implant sites at 0, 1, 2, 4-, 7-, 10-, and 14-days post-surgery. In vivo mineralization of excised subcutaneous implants can also be measured using x-ray radiography, micro-computed tomography.

In a sterile surgical environment, after installing a polysulfone fixation plates onto femurs of equal numbers of male and female 13-week-old Sprague Dawley rats, 6 mm bilateral bone defects can be made to femurs using a bone saw. After creating the defects, 6 mm long treatment allograft prepared in house from previously deceased rats will be implanted. As negative controls, allografts with no protein and with BMP-2 specific affibody only can be implanted. As a positive control, allograft containing only BMP-2 can be implanted. The treatment allograft will contain BMP-2 bound fusion protein. X-ray and micro-CT can be performed on live rats weeks 2, 4, 8, and 12 to assess the amount of mineralization surrounding the fixation plate and within the defect area. Micro-CT can be performed on rats from weeks 2, 8 and 12.

Immunohistochemistry can be performed on excised tissue at the end of 12 weeks using H&E staining, Safranin O, Fast Green, and Picrosirius red. H&E staining is expected to reveal improved bone quality from a greater number of bone cell nuclei on scaffolds containing both fusion protein and BMP-2, compared to the scaffolds that contained BMP-2 and no fusion protein. Safranin O will reveal cartilage formation, while Picrosirius will reveal highly organized lamellar bone formation.

The above methods can be repeated for fusion proteins specific for any other therapeutic proteins, including VEGF, FGF-2, PDGF, GM-CSF, and IL-4, or any combinations thereof, such as a fusion protein specific for GM-CSF and a fusion protein specific for IL-4.

### Example 15: Release of BMP-2 In Vivo

To investigate the effects of both fusion proteins on BMP-2 retention in vivo, BMP-2 will be labeled with a near-infrared fluorophore (e.g., Vivotag 750 or Vivotag 800). The labeled BMP-2 will then be incubated with the fusion protein, and the complex will be loaded onto either a collagen sponge/hydrogel (for the fusion protein with a collagen binding domain) or hydroxyapatite chips or bone allografts (for the fusion protein with a hydroxyapatite binding domain). The materials will be implanted subcutaneously in the backs of rats, and BMP-2 fluorescence intensity will be measured using an In Vivo Imaging System (IVIS) at days 0, 1, 2, 4, 7, 14, and 21 post-implantation. The above methods can be repeated for fusion proteins specific for any other therapeutic proteins, including VEGF, FGF-2, PDGF, GM-CSF, and IL-4, or any combinations thereof, such as a fusion protein specific for GM-CSF and a fusion protein specific for IL-4.

### Example 16: Promoting Bone Formation In Vivo

To investigate the effects of both fusion proteins on BMP-2-induced bone formation in vivo, the materials described above will be implanted in the backs of rats for 6-8 weeks, then the materials will be explanted and evaluated for mineral formation using x-ray radiography and ex vivo micro-computed tomography. Mineralization can also be investigated in an 8-mm critically sized orthotopic femoral bone defect in rats. Femurs will be stabilized with a polysulfone fixation plate with metal risers prior to creation of the defect. Similar to previous studies, a polycaprolactone (PCL) mesh tube with laser-cut holes will be placed in the defect site, and collagen hydrogels or HA bone chips will be placed in the tube. Alternatively, dry collagen sponges or bulk bone allografts will be press-fit into the defect site. Biomaterials can be fabricated with 1) no BMP-2 or fusion proteins or affibodies, 2) BMP-2, 3) BMP-2 and affibodies with no collagen or hydroxyapatite binding domain, or 4) BMP-2 and fusion proteins. The amount of BMP-2 affibodies required for BMP-2 localization can be determined based on in vitro experiments. A sample size of at least 10 defects per group can be used to evaluate bone repair (e.g., bone volume and quality) via x-ray radiography, micro-computed tomography, and torsion testing for mechanical strength, e.g., over 12 weeks. Histology (hemotoxylin/eosin and safranin 0/fast green) will also be performed to evaluate morphology of regenerated tissue in the bone defect. The above methods can be repeated for fusion proteins specific for any other therapeutic proteins, including VEGF, FGF-2, PDGF, GM-CSF, and IL-4, or any combinations thereof, such as a fusion protein specific for GM-CSF and a fusion protein specific for IL-4.

Given the high stability of fusion proteins, fusion protein-coated allografts can be lyophilized to create a device similar to clinically used allografts. Immediately prior to use, recombinant therapeutic proteins such as BMP-2, IL-4, and GM-CSF can be applied to the allograft in a manner similar to the clinical procedure for applying BMP-2 on collagen sponges. The high affinities of these fusion proteins also enable the use of relatively low, safe doses of recombinant therapeutic proteins such as BMP-2, IL-4, and GM-CSF.

### REFERENCES

(1) Miyazono, K.; Kamiya, Y.; Morikawa, M. Bone Morphogenetic Protein Receptors and Signal Transduction. J. Biochem. (Tokyo) 2010, 147 (1), 35-51. https://doi.org/10.1093/jb/mvpl48.
(2) James, A. W.; LaChaud, G.; Shen, J.; Asatrian, G.; Nguyen, V.; Zhang, X.; Ting, K.; Soo, C. A Review of the Clinical Side Effects of Bone Morphogenetic Protein-2. Tissue Eng. Part B Rev. 2016, 22 (4), 284-297. https://doi.org/10.1089/ten.teb.2015.0357.
(3) Hiepen et al., BMP2-Induced Chemotaxis Requires PI3K P55γ/P110α-Dependent Phosphatidylinositol (3,4,5)-Triphosphate Production and LL5β Recruitment at the Cytocortex. BMC Biol. 2014, 12 (1), 43. https://doi.org/10.1186/1741-7007-12-43.
(4) Borgiani et al., Bone Morphogenetic Protein 2-Induced Cellular Chemotaxis Drives Tissue Patterning during Critical-Sized Bone Defect Healing: An in Silico Study. Biomech. Model. Mechanobiol. 2021, 20 (4), 1627-1644. https://doi.org/10.1007/s10237-021-01466-0.
(5) Lind, M.; Eriksen, E. F.; Bünger, C. Bone Morphogenetic Protein-2 but Not Bone Morphogenetic Protein-4 and -6 Stimulates Chemotactic Migration of Human Osteoblasts, Human Marrow Osteoblasts, and U2-OS Cells. Bone 1996, 18 (1), 53-57. https://doi.org/10.1016/8756-3282(95)00423-8.
(6) SUN, J.; LI, J.; LI, C.; YU, Y. Role of Bone Morphogenetic Protein-2 in Osteogenic Differentiation of Mesenchymal Stem Cells. Mol. Med. Rep. 2015, 12 (3), 4230-4237. https://doi.org/10.3892/mmr.2015.3954.
(7) Briquez, P. S.; Tsai, H.-M.; Watkins, E. A.; Hubbell, J. A. Engineered Bridge Protein with Dual Affinity for Bone Morphogenetic Protein-2 and Collagen Enhances Bone Regeneration for Spinal Fusion. Sci. Adv. 2021, 7 (24), eabh4302. https://doi.org/10.1126/sciadv.abh4302.
(8) Kim, H. D.; Valentini, R. F. Retention and Activity of BMP-2 in Hyaluronic Acid-Based Scaffolds in Vitro. J. Biomed. Mater. Res. 2002, 59 (3), 573-584. https://doi.org/10.1002/jbm.10011.
(9) Noshi, T.; Yoshikawa, T.; Dohi, Y.; Ikeuchi, M.; Horiuchi, K.; Ichijima, K.; Sugimura, M.; Yonemasu, K.; Ohgushi, H. Recombinant Human Bone Morphogenetic Protein-2 Potentiates the in Vivo Osteogenic Ability of Marrow/Hydroxyapatite Composites. Artif Organs 2001, 25 (3), 201-208. https://doi.org/10.1046/j.1525-1594.2001.025003201.x.
(10) Kamal, A. F.; Siahaan, O. S. H.; Fiolin, J. Various Dosages of BMP-2 for Management of Massive Bone Defect in Sprague Dawley Rat. Arch. Bone Jt. Surg. 2019, 7 (6), 498-505.
(11) Hettiaratchi et al., Enhanced in Vivo Retention of Low Dose BMP-2 via Heparin Microparticle Delivery Does Not Accelerate Bone Healing in a Critically Sized Femoral Defect. Acta Biomater. 2017, 59, 21-32. https://doi.org/10.1016/j.actbio.2017.06.028.
(12) Rihn et al., Complications Associated with Single-Level Transforaminal Lumbar Interbody Fusion. Spine J. Off J. North Am. Spine Soc. 2009, 9 (8), 623-629. https://doi.org/10.1016/j.spinee.2009.04.004.
(13) Weber et al., The Effects of PEG Hydrogel Crosslinking Density on Protein Diffusion and Encapsulated Islet Survival and Function. J. Biomed. Mater. Res. A 2009, 90 (3), 720-729. https://doi.org/10.1002/jbm.a.32134.
(14) Sheth et al., Predicting Drug Release From Degradable Hydrogels Using Fluorescence Correlation Spectroscopy and Mathematical Modeling. Front. Bioeng. Biotechnol. 2019, 7.
(15) Dorogin et al., Biomaterials for Protein Delivery for Complex Tissue Healing Responses. Biomater. Sci. 2021, 9 (7), 2339-2361. https://doi.org/10.1039/DOBM01804J.
(16) Hettiaratchi et al., Competitive Protein Binding Influences Heparin-Based Modulation of Spatial Growth Factor Delivery for Bone Regeneration. Tissue Eng. Part A 2017, 23 (13-14), 683-695. https://doi.org/10.1089/ten.tea.2016.0507.
(17) Rajabi et al., Effect of Chemical Immobilization of SDF-1α into Muscle-Derived Scaffolds on Angiogenesis and Muscle Progenitor Recruitment. J. Tissue Eng. Regen. Med. 2018, 12 (1), e438-e450. https://doi.org/10.1002/term.2479.
(18) Bax et al., Impact of UV- and Carbodiimide-Based Crosslinking on the Integrin-Binding Properties of Collagen-Based Materials. Acta Biomater. 2019, 100, 280-291. https://doi.org/10.1016/j.actbio.2019.09.046.
(19) Lai, J.-Y.; Luo, L.-J.; Ma, D. Effect of Cross-Linking Density on the Structures and Properties of Carbodiimide-Treated Gelatin Matrices as Limbal Stem Cell Niches. Int. J. Mol. Sci. 2018, 19 (11), 3294. https://doi.org/10.3390/ijms19113294.
(20) Yamamoto, S.; Iwamaru, Y.; Shimizu, Y.; Ueda, Y.; Sato, M.; Yamaguchi, K.; Nakanishi, J. Epidermal Growth Factor-Nanoparticle Conjugates Change the Activity from Anti-Apoptotic to pro-Apoptotic at Membrane Rafts. Acta Biomater. 2019, 88, 383-391. https://doi.org/10.1016/j.actbio.2019.02.026.
(21) Hettiaratchi et al., Heparin Microparticle Effects on Presentation and Bioactivity of Bone Morphogenetic Protein-2. Biomaterials 2014, 35 (25), 7228-7238. https://doi.org/10.1016/j.biomaterials.2014.05.011.
(22) Benoit, D. S. W.; Durney, A. R.; Anseth, K. S. The Effect of Heparin-Functionalized PEG Hydrogels on Three-Dimensional Human Mesenchymal Stem Cell Osteogenic Differentiation. Biomaterials 2007, 28 (1), 66-77. https://doi.org/10.1016/j.biomaterials.2006.08.033.
(23) Sakiyama-Elbert, S. E.; Hubbell, J. A. Controlled Release of Nerve Growth Factor from a Heparin-Containing Fibrin-Based Cell Ingrowth Matrix. J. Controlled Release 2000, 69 (1), 149-158. https://doi.org/10.1016/S0168-3659(00)00296-0.
(24) Bhattacharya et al., Sulfation Modulates the Targeting Properties of Hyaluronic Acid to P-Selectin and CD44. ACS Biomater. Sci. Eng. 2020, 6 (6), 3585-3598. https://doi.org/10.1021/acsbiomaterials.0c00115.
(25) Limasale et al., Tuning the Local Availability of VEGF within Glycosaminoglycan-Based Hydrogels to Modulate Vascular Endothelial Cell Morphogenesis. Adv. Funct. Mater. 2020, 2000068. https://doi.org/10.1002/adfm.202000068.
(26) Abune, L.; Wang, Y. Affinity Hydrogels for Protein Delivery. Trends Pharmacol. Sci. 2021, 42 (4), 300-312. https://doi.org/10.1016/j.tips.2021.01.005.
(27) Wang et al., Covalent Immobilization of Biomolecules on Stent Materials through Mussel Adhesive Protein Coating to Form Biofunctional Films. Mater. Sci. Eng. C 2020, 106, 110187. https://doi.org/10.1016/j.msec.2019.110187.
(28) Mardilovich et al., Design of a Novel Fibronectin-Mimetic Peptide-Amphiphile for Functionalized Biomaterials. Langmuir 2006, 22 (7), 3259-3264. https://doi.org/10.1021/la052756n.
(29) Crispim et al., Bioactive Tape With BMP-2 Binding Peptides Captures Endogenous Growth Factors and Accelerates Healing After Anterior Cruciate Ligament Reconstruction. Am. J. Sports Med. 2018, 46 (12), 2905-2914. https://doi.org/10.1177/0363546518787507.
(30) Soontornworajit et al., Hydrogel Functionalization with DNA Aptamers for Sustained PDGF-BB Release. Chem. Commun. 2010, 46 (11), 1857-1859. https://doi.org/10.1039/B924909E.
(31) Hettiaratchi, M. H.; Krishnan, L.; Rouse, T.; Chou, C.; McDevitt, T. C.; Guldberg, R. E. Heparin-Mediated Delivery of Bone Morphogenetic Protein-2 Improves Spatial Localization of Bone Regeneration. Sci. Adv. 2020, 6 (1), eaay1240. https://doi.org/10.1126/sciadv.aayl240.
(32) Peysselon, F.; Ricard-Blum, S. Heparin-Protein Interactions: From Affinity and Kinetics to Biological Roles. Application to an Interaction Network Regulating Angiogenesis. Matrix Biol. 2014, 35, 73-81. https://doi.org/10.1016/j.matbio.2013.11.001.
(33) Shaughnessy, A. F. Monoclonal Antibodies: Magic Bullets with a Hefty Price Tag. BMJ2012, 345 (dec12 1), e8346-e8346. https://doi.org/10.1136/bmj.e8346.
(34) Sexton et al., Fluorescent Affibody Peptide Penetration in Glioma Margin Is Superior to Full Antibody. PLoS ONE 2013, 8 (4), e60390. https://doi.org/10.1371/journal.pone.0060390.
(35) Sachdeva et al., A Rational Approach for Creating Peptides Mimicking Antibody Binding. Sci. Rep. 2019, 9 (1), 997. https://doi.org/10.1038/s41598-018-37201-6.
(36) Trier, N.; Hansen, P.; Houen, G. Peptides, Antibodies, Peptide Antibodies and More. Int. J. Mol. Sci. 2019, 20 (24), 6289. https://doi.org/10.3390/ijms20246289.
(37) Munoz, E. M.; Linhardt, R. J. Heparin-Binding Domains in Vascular Biology. Arterioscler. Thromb. Vasc. Biol. 2004, 24 (9), 1549-1557. https://doi.org/10.1161/01.ATV.0000137189.22999.3f.
(38) Ren, C.; Wen, X.; Mencius, J.; Quan, S. Selection and Screening Strategies in Directed Evolution to Improve Protein Stability. Bioresour. Bioprocess. 2019, 6 (1), 53. https://doi.org/10.1186/s40643-019-0288-y.
(39) Teal et al., Directed Evolution Enables Simultaneous Controlled Release of Multiple Therapeutic Proteins from Biopolymer-Based Hydrogels. Adv. Mater. n/a (n/a), 2202612. https://doi.org/10.1002/adma.202202612.
(40) Bostock et al., Affibody-Mediated Controlled Release of Fibroblast Growth Factor 2. J. Controlled Release 2022, 350, 815-828. https://doi.org/10.1016/j.jconrel.2022.09.004.
(41) Wang et al., An Injectable PEG Hydrogel Controlling Neurotrophin-3 Release by Affinity Peptides. J. Controlled Release 2021, 330, 575-586. https://doi.org/10.1016/j.jconrel.2020.12.045.
(42) Löfblom et al., Affibody Molecules: Engineered Proteins for Therapeutic, Diagnostic and Biotechnological Applications. FEBS Lett. 2010, 584 (12), 2670-2680. https://doi.org/10.1016/j.febslet.2010.04.014.
(43) Woldring, D. R.; Holec, P. V.; Stern, L. A.; Du, Y.; Hackel, B. J. A Gradient of Sitewise Diversity Promotes Evolutionary Fitness for Binder Discovery in a Three-Helix Bundle Protein Scaffold. Biochemistry 2017, 56 (11), 1656-1671. https://doi.org/10.1021/acs.biochem.6b01142.
(44) Frejd, F. Y.; Kim, K.-T. Affibody Molecules as Engineered Protein Drugs. Exp. Mol. Med. 2017, 49 (3), e306-e306. https://doi.org/10.1038/emm.2017.35.
(45) Alexis et al., HER-2-Targeted Nanoparticle-Affibody Bioconjugates for Cancer Therapy. Chem Med Chem 2008, 3 (12), 1839-1843. https://doi.org/10.1002/cmdc.200800122.
(46) Persson et al., Discovery, Optimization and Biodistribution of an Affibody Molecule for Imaging of CD69. Sci. Rep. 2021, 11 (1), 19151. https://doi.org/10.1038/s41598-021-97694-6.
(47) Güler et al., Increasing Thermal Stability and Improving Biodistribution of VEGFR2-Binding Affibody Molecules by a Combination of in Silico and Directed Evolution Approaches. Sci. Rep. 2020, 10 (1), 18148. https://doi.org/10.1038/s41598-020-74560-5.
(48) Case, B. A.; Kruziki, M. A.; Stem, L. A.; Hackel, B. J. Evaluation of Affibody Charge Modification Identified by Synthetic Consensus Design in Molecular PET Imaging of Epidermal Growth Factor Receptor. Mol. Syst. Des. Eng. 2018, 3 (1), 171-182. https://doi.org/10.1039/C7ME00095B.
(49) Hackel, B. J.; Kapila, A.; Dane Wittrup, K. Picomolar Affinity Fibronectin Domains Engineered Utilizing Loop Length Diversity, Recursive Mutagenesis, and Loop Shuffling. J. Mol. Biol. 2008, 381 (5), 1238-1252. https://doi.org/10.1016/j.jmb.2008.06.051.
(50) Chao et al., Isolating and Engineering Human Antibodies Using Yeast Surface Display. Nat. Protoc. 2006, 1 (2), 755-769. https://doi.org/10.1038/nprot.2006.94.
(51) Mahmood, T.; Yang, P.-C. Western Blot: Technique, Theory, and Trouble Shooting. North Am. J. Med. Sci. 2012, 4 (9), 429-434. https://doi.org/10.4103/1947-2714.100998.
(52) Stern et al., Titratable Avidity Reduction Enhances Affinity Discrimination in Mammalian Cellular Selections of Yeast-Displayed Ligands. ACS Comb. Sci. 2017, 19 (5), 315-323. https://doi.org/10.1021/acscombsci.6b00191.
(53) Colabroy, K. L.; Mayer, K. Benchtop Immobilized Metal Affinity Chromatography, Reconstitution and Assay of a Polyhistidine Tagged Metalloenzyme for the Undergraduate Laboratory. J. Vis. Exp. JoVE 2018, No. 138, 58012. https://doi.org/10.3791/58012.
(54) Robichon, C.; Luo, J.; Causey, T. B.; Benner, J. S.; Samuelson, J. C. Engineering Escherichia Coli BL21(DE3) Derivative Strains To Minimize E. Coli Protein Contamination after Purification by Immobilized Metal Affinity Chromatography v. Appl. Environ. Microbiol. 2011, 77 (13), 4634-4646. https://doi.org/10.1128/AEM.00119-11.
(55) Asiimwe et al., Recent Advances in Protein Modifications Techniques for the Targeting N-Terminal Cysteine. Pept. Sci. 2022, 114 (3), e24235. https://doi.org/10.1002/pep2.24235.
(56) Phelps et al., Maleimide Cross-Linked Bioactive PEG Hydrogel Exhibits Improved Reaction Kinetics and Cross-Linking for Cell Encapsulation and In Situ Delivery. Adv. Mater. 2012, 24 (1),
64-70. https://doi.org/10.1002/adma.201103574.
(57) Fisher, S. A.; Baker, A. E. G.; Shoichet, M. S. Designing Peptide and Protein Modified Hydrogels: Selecting the Optimal Conjugation Strategy. J. Am. Chem. Soc. 2017, 139 (22), 7416-7427. https://doi.org/10.1021/jacs.7b00513.
(58) Arbour et al., Recent Advances in the Synthesis of C-Terminally Modified Peptides. Org. Biomol. Chem. 2020, 18 (37), 7253-7272. https://doi.org/10.1039/d0ob01417f.
(59) Benavides et al., Poly(Dehydroalanine): Synthesis, Properties, and Functional Diversification of a Fluorescent Polypeptide. J. Am. Chem. Soc. 2022, 144 (9), 4214-4223. https://doi.org/10.1021/jacs.2c00383.
(60) Nielsen, R. W.; Tachibana, C.; Hansen, N. E.; Winther, J. R. Trisulfides in Proteins. Antioxid. Redox Signal. 2011, 15 (1), 67-75. https://doi.org/10.1089/ars.2010.3677.
(61) Zeng, J.; Davies, M. J. Evidence for the Formation of Adducts and S-(Carboxymethyl)Cysteine on Reaction of α-Dicarbonyl Compounds with Thiol Groups on Amino Acids, Peptides, and Proteins. Chem. Res. Toxicol. 2005, 18 (8), 1232-1241. https://doi.org/10.1021/tx050074u.
(62) Greenfield, N. J. Using Circular Dichroism Spectra to Estimate Protein Secondary Structure. Nat. Protoc. 2006, 1 (6), 2876-2890. https://doi.org/10.1038/nprot.2006.202.
(63) Rodger, A.; Marshall, D. Beginners Guide to Circular Dichroism. The Biochemist 2021, 43 (2), 58-64. https://doi.org/10.1042/bio_2020-105.
(64) Mirdita et al., ColabFold: Making Protein Folding Accessible to All. Nat. Methods 2022. https://doi.org/10.1038/s41592-022-01488-1.
(65) Mirdita, M.; Steinegger, M.; S″oding, J. MMseqs2 Desktop and Local Web Server App for Fast, Interactive Sequence Searches. Bioinformatics 2019, 35 (16), 2856-2858. https://doi.org/10.1093/bioinformatics/bty1057.
(66) Mirdita et al., Uniclust Databases of Clustered and Deeply Annotated Protein Sequences and Alignments. Nucleic Acids Res 2017, 45 (D1), D170-D176. https://doi.org/10.1093/nar/gkw1081.
(67) Evans et al., Protein Complex Prediction with AlphaFold-Multimer. bioRxiv 2021. https://doi.org/10.1101/2021.10.04.463034v1.
(68) Mitchell et al., The Microbiome Analysis Resource in 2020. Nucleic Acids Res 2019. https://doi.org/10.1093/nar/gkz1035.
(69) Jumper et al., Highly Accurate Protein Structure Prediction with AlphaFold. Nature 2021, 596 (7873), 583-589. https://doi.org/10.1038/s41586-021-03819-2.
(70) Nivón, L. G.; Moretti, R.; Baker, D. A Pareto-Optimal Refinement Method for Protein Design Scaffolds. PLOS ONE 2013, 8 (4), e59004. https://doi.org/10.1371/journal.pone.0059004.
(71) Tyka, M. D.; Keedy, D. A.; André, I.; DiMaio, F.; Song, Y.; Richardson, D. C.; Richardson, J. S.; Baker, D. Alternate States of Proteins Revealed by Detailed Energy Landscape Mapping. J. Mol. Biol. 2011, 405 (2), 607-618. https://doi.org/10.1016/j.jmb.2010.11.008.
(72) Conway et al., Relaxation of Backbone Bond Geometry Improves Protein Energy Landscape Modeling. Protein Sci. Publ. Protein Soc. 2014, 23 (1), 47-55. https://doi.org/10.1002/pro.2389.
(73) Pierce et al., ZDOCK Server: Interactive Docking Prediction of Protein-Protein Complexes and Symmetric Multimers. Bioinformatics 2014, 30 (12), 1771-1773. https://doi.org/10.1093/bioinformatics/btu097.
(74) Schrödinger, LLC. The PyMOL Molecular Graphics System, Version 1.8, 2015.
(75) Rahman et al., TGF-β/BMP Signaling and Other Molecular Events: Regulation of Osteoblastogenesis and Bone Formation. Bone Res. 2015, 3, 15005. https://doi.org/10.1038/boneres.2015.5.
(76) Maguire et al., Perturbing the Energy Landscape for Improved Packing during Computational Protein Design. Proteins 2021, 89 (4), 436-449. https://doi.org/10.1002/prot.26030.
(77) Hagemans et al., A Script to Highlight Hydrophobicity and Charge on Protein Surfaces. Front. Mol. Biosci. 2015, 2.
(78) Martino et al., Growth Factors Engineered for Super-Affinity to the Extracellular Matrix Enhance Tissue Healing. Science 2014, 343 (6173), 885-888. https://doi.org/10.1126/science.1247663.
(79) Boraschi-Diaz, I.; Wang, J.; Mort, J. S.; Komarova, S. V. Collagen Type I as a Ligand for Receptor-Mediated Signaling. Front. Phys. 2017, 5.
(80) Kotzsch et al., Structure Analysis of Bone Morphogenetic Protein-2 Type I Receptor Complexes Reveals a Mechanism of Receptor Inactivation in Juvenile Polyposis Syndrome*. J. Biol. Chem. 2008, 283 (9), 5876-5887. https://doi.org/10.1074/jbc.M706029200.
(81) Kirsch, T.; Nickel, J.; Sebald, W. BMP-2 Antagonists Emerge from Alterations in the Low-Affinity Binding Epitope for Receptor BMPR-II. EMBO J. 2000, 19 (13), 3314-3324. https://doi.org/10.1093/emboj/19.13.3314.
(82) Katagiri, T.; Tsukamoto, S. The Unique Activity of Bone Morphogenetic Proteins in Bone: A Critical Role of the Smad Signaling Pathway. Biol. Chem. 2013, 394 (6), 703-714. https://doi.org/10.1515/hsz-2012-0310.
(83) Karim et al., Heterodimer-Heterotetramer Formation Mediates Enhanced Sensor Activity in a Biophysical Model for BMP Signaling. PLoS Comput. Biol. 2021, 17 (9), e1009422. https://doi.org/10.1371/journal.pcbi.1009422.
(84) Liu et al., Myoblast Sensitivity and Fibroblast Insensitivity to Osteogenic Conversion by BMP-2 Correlates with the Expression of Bmpr-1a. BMC Musculoskelet. Disord. 2009, 10 (1), 51. https://doi.org/10.1186/1471-2474-10⁻⁵¹.
(85) Song, R.; Wang, D.; Zeng, R.; Wang, J. Synergistic Effects of Fibroblast Growth Factor-2 and Bone Morphogenetic Protein-2 on Bone Induction. Mol. Med. Rep. 2017, 16 (4), 4483-4492. https://doi.org/10.3892/mmr.2017.7183.
(86) Zernik, J.; Twarog, K.; Upholt, W. B. Regulation of Alkaline Phosphatase and Alpha 2(I) Procollagen Synthesis during Early Intramembranous Bone Formation in the Rat Mandible. Differ. Res. Biol. Divers. 1990, 44 (3), 207-215. https://doi.org/10.1111/j.1432-0436.1990.tb00619.x.
(87) Sepulveda, J. L. Chapter 10—Challenges in Routine Clinical Chemistry Analysis: Proteins and Enzymes. In Accurate Results in the Clinical Laboratory (Second Edition); Dasgupta, A., Sepulveda, J. L., Eds.; Elsevier, 2019; pp 141-163. https://doi.org/10.1016/B978-0-12-813776-5.00010-8.
(88) Zhang et al., Exploring the Tunable Excitation of QDs to Maximize the Overlap with the Absorber for Inner Filter Effect-Based Phosphorescence Sensing of Alkaline Phosphatase. Nanoscale 2017, 9 (40), 15606-15611. https://doi.org/10.1039/C7NR03673F.
(89) Friess et al., Characterization of Absorbable Collagen Sponges as RhBMP-2 Carriers. Int. J. Pharm. 1999, 187 (1), 91-99. https://doi.org/10.1016/S0378-5173(99)00174-X.
(90) Yang, H. S.; La, W.-G.; Cho, Y.-M.; Shin, W.; Yeo, G.-D.; Kim, B.-S. Comparison between Heparin-Conjugated Fibrin and Collagen Sponge as Bone Morphogenetic Protein-2 Carriers for Bone Regeneration. Exp. Mol. Med. 2012, 44 (5), 350-355. https://doi.org/10.3858/emm.2012.44.5.039.
(91) Jansen, L. E.; Negrón-Piñeiro, L. J.; Galarza, S.; Peyton, S. R. Control of Thiol-Maleimide Reaction Kinetics in PEG Hydrogel Networks. Acta Biomater. 2018, 70, 120-128. https://doi.org/10.1016/j.actbio.2018.01.043.
(92) Guo et al., Regulating the Homogeneity of Thiol-Maleimide Michael-Type Addition-Based Hydrogels Using Amino Biomolecules. Gels 2021, 7(4), 206. https://doi.org/10.3390/gels7040206.
(93) Abouelmagd et al., Release Kinetics Study of Poorly Water-Soluble Drugs from Nanoparticles: Are We Doing It Right?. ACS Publications. https://doi.org/10.1021/mp500817h.
(94) Jensen et al., In Vitro Release Studies of Insulin from Lipid Implants in Solution and in a Hydrogel Matrix Mimicking the Subcutis. Eur. J. Pharm. Sci. 2016, 81, 103-112. https://doi.org/10.1016/j.ejps.2015.10.011.
(95) Gstraunthaler et al., A Plea to Reduce or Replace Fetal Bovine Serum in Cell Culture Media. Cytotechnology 2013, 65 (5), 791-793. https://doi.org/10.1007/s10616-013-9633-8.
(96) Metters, A.; Hubbell, J. Network Formation and Degradation Behavior of Hydrogels Formed by Michael-Type Addition Reactions. Biomacromolecules 2005, 6 (1), 290-301. https://doi.org/10.1021/bm049607o.
(97) Mellott, M. B.; Searcy, K.; Pishko, M. V. Release of Protein from Highly Cross-Linked Hydrogels of Poly(Ethylene Glycol) Diacrylate Fabricated by UV Polymerization. Biomaterials 2001, 22 (9), 929-941. https://doi.org/10.1016/s0142-9612(00)00258-1.
(98) Leach, J. B.; Schmidt, C. E. Characterization of Protein Release from Photocrosslinkable Hyaluronic Acid-Polyethylene Glycol Hydrogel Tissue Engineering Scaffolds. Biomaterials 2005, 26 (2), 125-135. https://doi.org/10.1016/j.biomaterials.2004.02.018.
(99) Zustiak, S. P.; Leach, J. B. Characterization of Protein Release from Hydrolytically Degradable Poly(Ethylene Glycol) Hydrogels. Biotechnol. Bioeng. 2011, 108 (1), 197-206. https://doi.org/10.1002/bit.22911.
(100) Wang, Y.; Delgado-Fukushima, E.; Fu, R. X.; Doerk, G. S.; Kim, J. Controlling Drug Absorption, Release and Erosion of Photopatterned Protein Engineered Hydrogels.
(101) Vermonden, T.; Censi, R.; Hennink, W. E. Hydrogels for Protein Delivery. Chem. Rev. 2012, 112 (5), 2853-2888. https://doi.org/10.1021/cr200157d.
(102) Gregoritza, M.; Messmann, V.; M. Goepferich, A.; P. Brandl, F. Design of Hydrogels for Delayed Antibody Release Utilizing Hydrophobic Association and Diels—Alder Chemistry in Tandem. J. Mater. Chem. B 2016, 4 (19), 3398-3408. https://doi.org/10.1039/C6TB00223D.
(103) Quaas, B.; Burmeister, L.; Li, Z.; Satalov, A.; Behrens, P.; Hoffmann, A.; Rinas, U. Stability and Biological Activity of E. Coli Derived Soluble and Precipitated Bone Morphogenetic Protein-2. Pharm. Res. 2019, 36 (12), 184. https://doi.org/10.1007/s11095-019-2705-5.
(104) Sundermann, J.; Zagst, H.; Kuntsche, J.; Watzig, H.; Bunjes, H. Bone Morphogenetic Protein 2 (BMP-2) Aggregates Can Be Solubilized by Albumin—Investigation of BMP-2 Aggregation by Light Scattering and Electrophoresis. Pharmaceutics 2020, 12 (12), 1143. https://doi.org/10.3390/pharmaceutics12121143.
(105) Ritger, P. L.; Peppas, N. A. A Simple Equation for Description of Solute Release II. Fickian and Anomalous Release from Swellable Devices. J. Controlled Release 1987, 5 (1), 37-42. https://doi.org/10.1016/0168-3659(87)90035-6.
(106) Lowe, S.; O'Brien-Simpson, N. M.; Connal, L. A. Antibiofouling Polymer Interfaces: Poly(Ethylene Glycol) and Other Promising Candidates. Polym. Chem. 2014, 6 (2), 198-212. https://doi.org/10.1039/C4PY01356E.
(107) Kuo, J.-H. S. Effect of Pluronic-Block Copolymers on the Reduction of Serum-Mediated Inhibition of Gene Transfer of Polyethyleneimine—DNA Complexes. Biotechnol. Appl. Biochem. 2003, 37 (3), 267-271. https://doi.org/10.1042/BA20020123.
(108) Fung, S. L.; Wu, X.; Maceren, J. P.; Mao, Y.; Kohn, J. In Vitro Evaluation of Recombinant Bone Morphogenetic Protein-2 Bioactivity for Regenerative Medicine. Tissue Eng. Part C Methods 2019, 25 (9), 553-559. https://doi.org/10.1089/ten.tec.2019.0156.
(109) Hettiaratchi, M. H.; Krishnan, L.; Rouse, T.; Chou, C.; McDevitt, T. C.; Guldberg, R. E. Heparin-Mediated Delivery of Bone Morphogenetic Protein-2 Improves Spatial Localization of Bone Regeneration. Sci. Adv. 2020, 6 (1), eaay1240. https://doi.org/10.1126/sciadv.aayl240.
(110) Jung, T.; Lee, J. H.; Park, S.; Kim, Y.-J.; Seo, J.; Shim, H.-E.; Kim, K.-S.; Jang, H.-S.; Chung,
H.-M.; Oh, S.-G.; Moon, S.-H.; Kang, S.-W. Effect of BMP-2 Delivery Mode on Osteogenic Differentiation of Stem Cells. Stem Cells Int. 2017, 2017, 7859184. https://doi.org/10.1155/2017/7859184.
(111) Zhao, B.; Katagiri, T.; Toyoda, H.; Takada, T.; Yanai, T.; Fukuda, T.; Chung, U.; Koike, T.; Takaoka, K.; Kamijo, R. Heparin Potentiates the in Vivo Ectopic Bone Formation Induced by Bone Morphogenetic Protein-2 *. J. Biol. Chem. 2006, 281 (32), 23246-23253. https://doi.org/10.1074/jbc.M511039200.
(112) Van Hove, A. H.; Beltejar, M.-J.; Benoit, D. S. W. Development and in Vitro Assessment of Enzymatically-Responsive Poly(Ethylene Glycol) Hydrogels for the Delivery of Therapeutic Peptides. Biomaterials 2014, 35 (36), 9719-9730. https://doi.org/10.1016/j.biomaterials.2014.08.019.
(113) Shih, H.; Lin, C.-C. Crosslinking and Degradation of Step-Growth Hydrogels Formed by Thiol-Ene Photo-Click Chemistry. Biomacromolecules 2012, 13 (7), 2003-2012. https://doi.org/10.1021/bm300752j.
(114) Gawade, P. M.; Shadish, J. A.; Badeau, B. A.; DeForest, C. A. Logic-Based Delivery of Site-Specifically Modified Proteins from Environmentally Responsive Hydrogel Biomaterials. Adv. Mater. 2019, 31 (33), 1902462. https://doi.org/10.1002/adma.201902462.
(115) Marty, M. T.; Baldwin, A. J.; Marklund, E. G.; Hochberg, G. K. A.; Benesch, J. L. P.; Robinson, C. V. Bayesian Deconvolution of Mass and Ion Mobility Spectra: From Binary Interactions to Polydisperse Ensembles. Anal. Chem. 2015, 87(8), 4370-4376. https://doi.org/10.1021/acs.analchem.5b00140.
(116) Khatib, F.; Cooper, S.; Tyka, M. D.; Xu, K.; Makedon, I.; Popović, Z.; Baker, D.; Players, F. Algorithm Discovery by Protein Folding Game Players. Proc. Natl. Acad. Sci. 2011, 108 (47), 18949-18953. https://doi.org/10.1073/pnas.1115898108.
(117) Keller, S.; Nickel, J.; Zhang, J.-L.; Sebald, W.; Mueller, T. D. Molecular Recognition of BMP-2 and BMP Receptor IA. Nat. Struct. Mol. Biol. 2004, 11 (5), 481-488. https://doi.org/10.1038/nsmb756.
(118) Guo, J.; Liu, B.; Thorikay, M.; Yu, M.; Li, X.; Tong, Z.; Salmon, R. M.; Read, R. J.; ten Dijke, P.; Morrell, N. W.; Li, W. Crystal Structures of BMPRII Extracellular Domain in Binary and Ternary Receptor Complexes with BMP10. Nat. Commun. 2022, 13 (1), 2395. https://doi.org/10.1038/s41467-022-30111-2.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A dual-affinity fusion protein, comprising an affibody sequence, a linker, and a localization domain, wherein the affibody sequence is specific for a therapeutic protein, and the localization domain is specific for a structural bone component.

2. The dual-affinity fusion protein of claim 1, wherein the therapeutic protein is bone morphogenetic protein 2 (BMP-2), vascular endothelial growth factor (VEGF), fibroblast growth factor 2 (FGF-2), platelet-derived growth factor (PDGF), granulocyte macrophage colony stimulating factor (GM-CSF), or interleukin-4 (IL-4).

3. The dual-affinity fusion protein of claim 1, wherein the structural bone component is present in a collagen scaffold, collagen sponge, bone void filler, or bone graft.

4. The dual-affinity fusion protein of claim 1, wherein the structural bone component is collagen, a calcium phosphate, or a calcium sulphate.

5. The dual-affinity fusion protein of claim 4, wherein:
the collagen is Type I collagen, Type II collagen, or Type III collagen, or
the calcium phosphate is hydroxyapatite (HA), β-tricalcium phosphate (β-TCP), α-tricalcium phosphate (α-TCP), calcium deficient hydroxyapatite (CDHA), tetracalcium phosphate (TTCP), amorphous calcium phosphate (ACP), dicalcium phosphate dihydrate (DCPD), or dicalcium phosphate anhydrous (DCPA).

6. The dual-affinity fusion protein of claim 1, wherein the linker is a flexible linker, a rigid linker, or a hybrid linker comprising a flexible and a rigid segments.

7. The dual-affinity fusion protein of claim 1, wherein the affibody sequence comprises at least 90% sequence identity to any one of SEQ ID NOs: 1-68 and 71-74, with or without the initial A.

8. The dual-affinity fusion protein of claim 1, wherein the localization domain comprises at least 90% sequence identity to any one of SEQ ID NOs: 75-79 and 127.

9. The dual-affinity fusion protein of claim 1, wherein the linker sequence comprises any one of SEQ ID NOs: 80-85 and 120-125.

10. The dual-affinity fusion protein of claim 1, wherein the affibody sequence is 58 to 65 amino acids in length.

11. The dual-affinity fusion protein of claim 1, wherein the affibody sequence comprises 1, 2, 3, 4, 5 or 6 conservative amino acid substitutions as compared to any of SEQ ID NOs: 1-68 and 71-74, with or without the initial A.

12. The dual-affinity fusion protein of claim 1, wherein the fusion protein comprises at least 90% sequence identity to any one of SEQ ID NOs: 87-112 and 126, or comprises 1, 2, 3, 4, 5 or 6 conservative amino acid substitutions as compared to any of SEQ ID NOs: 87-112 and 126.

13. A composition comprising:
the dual-affinity fusion protein of claim 1, and
a therapeutic protein;
wherein the affibody sequence in the dual-affinity fusion protein is specific for the therapeutic protein.

14. The composition of claim 13, wherein the dual-affinity fusion protein is non-covalently bound to the therapeutic protein through the affibody sequence.

15. The composition of claim 13, wherein the composition further comprises a structural bone component.

16. The composition of claim 15, wherein the dual-affinity fusion protein is non-covalently bound to the structural bone component through the localization domain.

17. The composition of claim 13, wherein the therapeutic protein is bone morphogenetic protein 2 (BMP-2), vascular endothelial growth factor (VEGF), fibroblast growth factor 2 (FGF-2), platelet-derived growth factor (PDGF), granulocyte macrophage colony stimulating factor (GM-CSF), or interleukin-4 (IL-4).

18. The composition of claim 15, wherein the structural bone component is present in a collagen scaffold, collagen sponge, bone void filler, or bone graft.

19. The composition of claim 15, wherein the structural bone component is collagen, a calcium phosphate, or a calcium sulphate.

20. The composition of claim 19, wherein:
the collagen is Type I collagen, Type II collagen, or Type III collagen, or
the calcium phosphate is hydroxyapatite (HA), β-tricalcium phosphate (β-TCP), α-tricalcium phosphate (α-TCP), calcium deficient hydroxyapatite (CDHA), tetracalcium phosphate (TTCP), amorphous calcium phosphate (ACP), dicalcium phosphate dihydrate (DCPD), or dicalcium phosphate anhydrous (DCPA).

21. A method of treating a wound, or a bone or cartilage injury or disease in a subject, comprising:
administering an effective amount of the dual-affinity fusion protein of claim 1 to the subject, thereby treating the bone or cartilage injury or disease.

22. The method of claim 21, wherein the bone injury or disease is a bone fracture or a degenerative bone disease.

23. The method of claim 21, wherein the administering is surgical administration or injection.


SEQUENCE LISTING (127 sequences)

SEQ ID NO: 1 - protein, 58 aa, synthetic construct
AEAKYYKEVSSAATQIRYLPNLTAFQKAAFYAALLDDPSQSSELLSEAKKLNDSQAPK

SEQ ID NO: 2 - protein, 58 aa, synthetic construct
AEAKYAKEQFNAYVVIFYLPNLTASQKAAFVDALSNDPSQSSELLSEAKKLNDSQAPK

SEQ ID NO: 3 - protein, 58 aa, synthetic construct
AEAKYYKEGDNAYNVIYGLPNLTRPQRLAFIVALFNDPSQSSELLSEAKKLNDSQAPK

SEQ ID NO: 4 - protein, 58 aa, synthetic construct
AEAKYNKEVTAAANSIWVLPNLTGDQKAAFFEALLDDPSQSSELLSEAKKLNDSQAPK

SEQ ID NO: 5 - protein, 58 aa, synthetic construct
AEAKYTKEGFDAYDVIDNLPNLTLDQRNAFVYALFNDPSQSSELLSEAKKLNDSQAPK

SEQ ID NO: 6 - protein, 58 aa, synthetic construct
AEAKYYKEWLDADMSIRSLPNLTGYQIRAFIAALGNDPSQSSELLSEAKKLNDSQAPK

SEQ ID NO: 7 - protein, 58 aa, synthetic construct
AEAKYYKERRAAAVVIFYLPNLTRVQKGAFIEALDDDPSQSSELLSEAKKLNDSQAPK

SEQ ID NO: 8 - protein, 58 aa, synthetic construct
AEAKYAKERLNAIYVINDLPNLTQGQRVAFARALYNDPSQSSELLSEAKKLNDSQAPK

SEQ ID NO: 9 - protein, 58 aa, synthetic construct; identical to SEQ ID NO: 2

SEQ ID NO: 10 - protein, 58 aa, synthetic construct
AEAKYYKEWVNAYDQIRVLPNLTRFQRLAFYRALYNDPSQSSELLSEAKKLNDSQAPK

SEQ ID NO: 11 - protein, 58 aa, synthetic construct; identical to SEQ ID NO: 6

SEQ ID NO: 12 - protein, 58 aa, synthetic construct
AEAKYTKELFNAVGEITALPNLTRYHLYAFYYALLNDPSQSSELLSEAKKLNDSQAPK

SEQ ID NO: 13 - protein, 58 aa, synthetic construct
AEAKYNKEWFAADLSIGFLPNLTLDQLYAFVFALYDDPSQSSELLSEAKKLNDSQAPK

SEQ ID NO: 14 - protein, 58 aa, synthetic construct
AEAKYAKEGLNAYLSIRWLPNLTGDQMYAFISALLDDPSQSSELLSEAKKLNDSQAPK

SEQ ID NO: 15 - protein, 58 aa, synthetic construct
AEAKYTKEGFNAYDEIDNLPNLTLDQRNAFVYALFNDPSQSSELLSEAKKLNDSQAPK

SEQ ID NO: 16 - protein, 58 aa, synthetic construct; identical to SEQ ID NO: 12

SEQ ID NO: 17 - protein, 58 aa, synthetic construct
AEAKYNKEVGTANFEIVLLPNLTLYQMLAFIKALVNDPSQSSELLSEAKKLNDSQAPK

SEQ ID NO: 18 - protein, 58 aa, synthetic construct
AEAKYNKEWYNAISVIFYLPNLTGFQRAAFVDALGDDPSQSSELLSEAKKLNDSQAPK

SEQ ID NO: 19 - protein, 58 aa, synthetic construct
AEAKYYKEGFYANFVIGALPNLTLVQRAAFYFALLNDPSQSSELLSEAKKLNDSQAPK

SEQ ID NO: 20 - protein, 58 aa, synthetic construct
AEAKYYKEGATAYRVIEYLPNLTGAQKAAFIDALYNDPSQSSELLSEAKKLNDSQAPK

SEQ ID NO: 21 - protein, 58 aa, synthetic construct; identical to SEQ ID NO: 5

SEQ ID NO: 22 - protein, 58 aa, synthetic construct
AEAKYNKEWYDAVFVIGSLPNLTEDQKDAFSDALVDDPSQSSELLSEAKKLNDSQAPK

SEQ ID NO: 23 - protein, 58 aa, synthetic construct
AEAKYYKEWNAAYVVINGLPNLTRRQREAFVHALVDDPSQSSELLSEAKKLNDSQAPK

SEQ ID NO: 24 - protein, 58 aa, synthetic construct
AEAKYYKERYAANYSIWVLPNLTLLQRFAFFFALSNDPSQSSELLSEAKKLNDSQAPK

SEQ ID NO: 25 - protein, 58 aa, synthetic construct
AEAKYAKELDDAFFEIASLPNLTGFQLHAFAVALGNDPSQSSELLSEAKKLNDSQAPK

SEQ ID NO: 26 - protein, 58 aa, synthetic construct
AEAKYNKERDSAYSVIWGLPNLTDSQKAAFGYALYNDPSQSSELLSEAKKLNDSQAPK

SEQ ID NO: 27 - protein, 58 aa, synthetic construct
AEAKYAKELEAANMVIVDLPNLTHGQKVAFLVALFNDPSQSSELLSEAKKLNDSQAPK

SEQ ID NO: 28 - protein, 58 aa, synthetic construct
AEAKYNKEWYDAILEIGFLPNLTGHQRDAFSDALVDDPSQSSELLSEAKKLNDSQAPK

SEQ ID NO: 29 - protein, 58 aa, synthetic construct
AEAKYNKEQDSAYSVIWGLPNLTESQKAAFGYALYDDPSQSSELLSEAKKLNDSQAPK

SEQ ID NO: 30 - protein, 58 aa, synthetic construct; identical to SEQ ID NO: 4

SEQ ID NO: 31 - protein, 58 aa, synthetic construct
AEAKYAKEWFYAYHVIYDLPNLTGFQKHAFYLALYDDPSQSSELLSEAKKLNDSQAPK

SEQ ID NO: 32 - protein, 58 aa, synthetic construct; identical to SEQ ID NO: 4

SEQ ID NO: 33 - protein, 58 aa, synthetic construct
AEAKYAKEGATAFGSIPYLPNLTDVQRYAFIVALLDDPSQSSELLSEAKKLNDSQAPK

SEQ ID NO: 34 - protein, 58 aa, synthetic construct
AEAKYTKEWYAAVVQIGYLPNLTAFQRAAFSFALSNDPSQSSELLSEAKKLNDSQAPK

SEQ ID NO: 35 - protein, 58 aa, synthetic construct
AEAKYTKERDDASLEIAYLPNLTPYQLMAFFFALSNDPSQSSELLSEAKKLNDSQAPK

SEQ ID NO: 36 - protein, 58 aa, synthetic construct
AEAKYAKEWTNAFVSIVCLPNLTAVQREAFVLALVDDPSQSSELLSEAKKLNDSQAPK

SEQ ID NO: 37 - protein, 58 aa, synthetic construct
AEAKYAKEWEDAINEIWCLPNLTEYQRIAFVSALYNDPSQSSELLSEAKKLNDSQAPK

SEQ ID NO: 38 - protein, 58 aa, synthetic construct
AEAKYAKELLNAFDEIYGLPNLTVGQRMAFCDALINDPSQSSELLSEAKKLNDSQAPK

SEQ ID NO: 39 - protein, 58 aa, synthetic construct
AEAKYYKEWYDAFVVIDALPNLTAYQREAFIFALVNDPSQSSELLSEAKKLNDSQAPK

SEQ ID NO: 40 - protein, 58 aa, synthetic construct
AEAKYYKEWVDAYLVIDSLPNLTRLQVEAFVFALVNDPSQSSELLSEAKKLNDSQAPK

SEQ ID NO: 41 - protein, 58 aa, synthetic construct
AEAKYTKEVDYAACVIAYLPNLTGVQVYAFYRALADDPSQSSELLSEAKKLNDSQAPK

SEQ ID NO: 42 - protein, 58 aa, synthetic construct
AEAKYTKEGSDAFDVIVLLPNLTRDQRDAFLYALLDDPSQSSELLSEAKKLNDSQAPK

SEQ ID NO: 43 - protein, 58 aa, synthetic construct
AEAKYAKEWLSADYVIICLPNLTLDQMVAFYDALFNDPSQSSELLSEAKKLNDSQAPK

SEQ ID NO: 44 - protein, 58 aa, synthetic construct
AEAKYNKEVFDADCSIWYLPNLTRYQISAFQSALDDDPSQSSELLSEAKKLNDSQAPK

SEQ ID NO: 45 - protein, 58 aa, synthetic construct
AEAKYTKEGCDAYTEIVDLPNLTGYQRRAFYWALENDPSQSSELLSEAKKLNDSQAPK

SEQ ID NO: 46 - protein, 58 aa, synthetic construct
AEAKYNKEMPDANCQIAFLPNLTQYQVPAFIYALCNDPSQSSELLSEAKKLNDSQAPK

SEQ ID NO: 47 - protein, 58 aa, synthetic construct
AEAKYNKEGEDATTQIGSLPNLTQAQKHAFAVALGNDPSQSSELLSEAKKLNDSQAPK

SEQ ID NO: 48 - protein, 58 aa, synthetic construct
AEAKYSKEGFYADWVIPVLPNLTRKQRVAFHDALHNDPSQSSELLSEAKKLNDSQAPK

SEQ ID NO: 49 - protein, 58 aa, synthetic construct
AEAKYAKEWLDAIDVIGYLPNLTDFQRGAFYDALNDDPSQSSELLSEAKKLNDSQAPK

SEQ ID NO: 50 - protein, 58 aa, synthetic construct
AEAKYYKEGYNAIVEIRCLPNLTDCQVAAFIDALDDDPSQSSELLSEAKKLNDSQAPK

SEQ ID NO: 51 - protein, 58 aa, synthetic construct
AEAKYAKELDAAYVVIYFLPNLTHCQMVAFLHALSDDPSQSSELLSEAKKLNDSQAPK

SEQ ID NO: 52 - protein, 58 aa, synthetic construct
AEAKYSKEVYSAYDVIFALPNLTQYQVLAFFDALCDDPSQSSELLSEAKKLNDSQAPK

SEQ ID NO: 53 - protein, 58 aa, synthetic construct
AEAKYAKERLTAVCSIVALPNLTEGQMVAFDDALHDDPSQSSELLSEAKKLNDSQAPK

SEQ ID NO: 54 - protein, 58 aa, synthetic construct
AEAKYAKEGFNAVNVIWPLPNLTADQVCAFICALADDPSQSSELLSEAKKLNDSQAPK

SEQ ID NO: 55 - protein, 58 aa, synthetic construct
AEAKYAKEGCTAFLEIAALPNLTGYQRDAFIEALFDDPSQSSELLSEAKKLNDSQAPK

SEQ ID NO: 56 - protein, 58 aa, synthetic construct; identical to SEQ ID NO: 42

SEQ ID NO: 57 - protein, 58 aa, synthetic construct
AEAKYYKEWDSASDSIGFLPNLTRAQMVAFFAALFNDPSQSSELLSEAKKLNDSQAPK

SEQ ID NO: 58 - protein, 58 aa, synthetic construct
AEAKYAHELWEADWEITNLPNLSPDQLMAFYMALWDDPSQSSELLSEAKKLNDSQAPK

SEQ ID NO: 59 - protein, 58 aa, synthetic construct
AEAKYAFELWEAQHEIQQLPNLRPDQIAAFAMALYDDPSQSSELLSEAKKLNDSQAPK

SEQ ID NO: 60 - protein, 58 aa, synthetic construct
AEAKYAKELDDASVEIWDLPNLTPCQKVAFFVALYDDPSQSSELLSEAKKLNDSQAPK

SEQ ID NO: 61 - protein, 60 aa, synthetic construct
AEAKYNKELDAADADVEIWLLPNLTLDQLLAFIAALFNDPSQSSELLSEAKKLNDSQAPK

SEQ ID NO: 62 - protein, 58 aa, synthetic construct
AEAKYTKELSDANAEIWSLPNLTVDQLVAFIFALWDDPSQSSELLSEAKKLNDSQAPK

SEQ ID NO: 63 - protein, 58 aa, synthetic construct
AEAKYSKEQSNAYASITDLPNLTRLQKLAFWVALFNDPSQSSELLSEAKKLNDSQAPK

SEQ ID NO: 64 - protein, 58 aa, synthetic construct
AERKYHWELLVAFMEIQSLPNLTKDQITQFMAALEDDPSQSSELLSEAKKLNDSQAPK

SEQ ID NO: 65 - protein, 65 aa, synthetic construct
AEAKYYKEVSSAATQIRYLPNLTAFQKAAFYAALLDDPSQSSELLSEAKKLNDSQAPKHHHHHHC

SEQ ID NO: 66 - protein, 65 aa, synthetic construct
AEAKYAKEQFNAYVVIFYLPNLTASQKAAFVDALSNDPSQSSELLSEAKKLNDSQAPKHHHHHHC

SEQ ID NO: 67 - protein, 65 aa, synthetic construct
AEAKYYKEGDNAYNVIYGLPNLTRPQRLAFIVALFNDPSQSSELLSEAKKLNDSQAPKHHHHHHC

SEQ ID NO: 68 - protein, 65 aa, synthetic construct
AEAKYTKELFNAVGEITALPNLTRYHLYAFYYALLNDPSQSSELLSEAKKLNDSQAPKHHHHHHC

SEQ ID NO: 69 - DNA, 20 bp, synthetic construct
  source 1..20: mol_type = other DNA
ccctcaacaactagcaaagg

SEQ ID NO: 70 - DNA, 24 bp, synthetic construct
  source 1..24: mol_type = other DNA
gcaaggcaatggttgaaatgtgta

SEQ ID NO: 71 - protein, 58 aa, synthetic construct
AEAKYNKEWYDAVFVIGSLPNLTEDQKAAFSDALVDDPSQSSELLSEAKKLNDSQAPK

SEQ ID NO: 72 - protein, 58 aa, synthetic construct
AEAKYNKEWYDAVFVIGSLPNLTEDQKDAFSAALVDDPSQSSELLSEAKKLNDSQAPK

SEQ ID NO: 73 - protein, 58 aa, synthetic construct
AEAKYNKEWYDAVFVIGSLPNLTEDQKDAFSDALVADPSQSSELLSEAKKLNDSQAPK

SEQ ID NO: 74 - protein, 58 aa, synthetic construct
AEEKYMMEAHWALMEILNLPNLHPCQQDAFWLALWDDPSQSSELLSEAKKLNDSQAPK

SEQ ID NO: 75 - protein, 7 aa, synthetic construct
TKKTLRT

SEQ ID NO: 76 - protein, 8 aa, synthetic construct
TKKLTLRT

SEQ ID NO: 77 - protein, 11 aa, synthetic construct
EEEEEEEDGEA

SEQ ID NO: 78 - protein, 6 aa, synthetic construct
GKNFQS

SEQ ID NO: 79 - protein, 7 aa, synthetic construct
SVSVSVK

SEQ ID NO: 80 - protein, 5 aa, synthetic construct
GGGGS

SEQ ID NO: 81 - protein, 15 aa, synthetic construct
GGGGSGGGGSGGGGS

SEQ ID NO: 82 - protein, 12 aa, synthetic construct
AEAAAKEAAAKA

SEQ ID NO: 83 - protein, 10 aa, synthetic construct
LAEAAAKAAA

SEQ ID NO: 84 - protein, 7 aa, synthetic construct
GGGAAAA

SEQ ID NO: 85 - protein, 11 aa, synthetic construct
EAAAKAGGGGS

SEQ ID NO: 86 - protein, 6 aa, synthetic construct
HHHHHH

SEQ ID NO: 87 - protein, 70 aa, synthetic construct
GTKKTLRTGGGGSEAKYYKEVSSAATQIRYLPNLTAFQKAAFYAALLDDPSQSSELLSEAKKLNDSQAPK

SEQ ID NO: 88 - protein, 78 aa, synthetic construct
GTKKLTLRTAEAAAKEAAAKAEAKYYKEVSSAATQIRYLPNLTAFQKAAFYAALLDDPSQSSELLSEAKKLNDSQAPK

SEQ ID NO: 89 - protein, 71 aa, synthetic construct
GTKKLTLRTGGGGSEAKYYKEVSSAATQIRYLPNLTAFQKAAFYAALLDDPSQSSELLSEAKKLNDSQAPK

SEQ ID NO: 90 - protein, 71 aa, synthetic construct
GTKKLTLRTGGGGSEAKYAKEQFNAYVVIFYLPNLTASQKAAFVDALSNDPSQSSELLSEAKKLNDSQAPK

SEQ ID NO: 91 - protein, 71 aa, synthetic construct
GTKKLTLRTGGGGSEAKYYKEGDNAYNVIYGLPNLTRPQRLAFIVALFNDPSQSSELLSEAKKLNDSQAPK

SEQ ID NO: 92 - protein, 71 aa, synthetic construct
GTKKLTLRTGGGGSEAKYTKELFNAVGEITALPNLTRYHLYAFYYALLNDPSQSSELLSEAKKLNDSQAPK

SEQ ID NO: 93 - protein, 71 aa, synthetic construct
GTKKLTLRTGGGGSEAKYNKEWFAADLSIGFLPNLTLDQLYAFVFALYDDPSQSSELLSEAKKLNDSQAPK

SEQ ID NO: 94 - protein, 71 aa, synthetic construct
GTKKLTLRTGGGGSEAKYAKEGLNAYLSIRWLPNLTGDQMYAFISALLDDPSQSSELLSEAKKLNDSQAPK

SEQ ID NO: 95 - protein, 73 aa, synthetic construct
GTKKLTLRTGGGGSEAKYNKELDAADADVEIWLLPNLTLDQLLAFIAALFNDPSQSSELLSEAKKLNDSQAPK

SEQ ID NO: 96 - protein, 71 aa, synthetic construct
GTKKLTLRTGGGGSEAKYTKELSDANAEIWSLPNLTVDQLVAFIFALWDDPSQSSELLSEAKKLNDSQAPK

SEQ ID NO: 97 - protein, 71 aa, synthetic construct
GTKKLTLRTGGGGSEEKYMMEAHWALMEILNLPNLHPCQQDAFWLALWDDPSQSSELLSEAKKLNDSQAPK

SEQ ID NO: 98 - protein, 71 aa, synthetic construct
GTKKLTLRTGGGGSEAKYYKEWDSASDSIGFLPNLTRAQMVAFFAALFNDPSQSSELLSEAKKLNDSQAPK

SEQ ID NO: 99 - protein, 71 aa, synthetic construct
GTKKLTLRTGGGGSEAKYAHELWEADWEITNLPNLSPDQLMAFYMALWDDPSQSSELLSEAKKLNDSQAPK

SEQ ID NO: 100 - protein, 71 aa, synthetic construct
GTKKLTLRTGGGGSEAKYAFELWEAQHEIQQLPNLRPDQIAAFAMALYDDPSQSSELLSEAKKLNDSQAPK

SEQ ID NO: 101 - protein, 71 aa, synthetic construct
GTKKLTLRTGGGGSEAKYAKELDDASVEIWDLPNLTPCQKVAFFVALYDDPSQSSELLSEAKKLNDSQAPK

SEQ ID NO: 102 - protein, 71 aa, synthetic construct
GTKKLTLRTGGGGSEAKYNKEWYDAVFVIGSLPNLTEDQKAAFSDALVDDPSQSSELLSEAKKLNDSQAPK

SEQ ID NO: 103 - protein, 71 aa, synthetic construct
GTKKLTLRTGGGGSEAKYNKEWYDAVFVIGSLPNLTEDQKDAFSAALVDDPSQSSELLSEAKKLNDSQAPK

SEQ ID NO: 104 - protein, 71 aa, synthetic construct
GTKKLTLRTGGGGSEAKYNKEWYDAVFVIGSLPNLTEDQKDAFSDALVADPSQSSELLSEAKKLNDSQAPK

SEQ ID NO: 105 - protein, 71 aa, synthetic construct
GTKKLTLRTGGGGSEAKYYKEGATAYRVIEYLPNLTGAQKAAFIDALYNDPSQSSELLSEAKKLNDSQAPK

SEQ ID NO: 106 - protein, 71 aa, synthetic construct
GTKKLTLRTGGGGSEAKYTKEGFDAYDVIDNLPNLTLDQRNAFVYALFNDPSQSSELLSEAKKLNDSQAPK

SEQ ID NO: 107 - protein, 71 aa, synthetic construct
GTKKLTLRTGGGGSEAKYNKEWYDAVFVIGSLPNLTEDQKDAFSDALVDDPSQSSELLSEAKKLNDSQAPK

SEQ ID NO: 108 - protein, 80 aa, synthetic construct
EEEEEEEDGEAAEAAAKEAAAKAEAKYYKEVSSAATQIRYLPNLTAFQKAAFYAALLDDPSQSSELLSEAKKLNDSQAPK

SEQ ID NO: 109 - protein, 78 aa, synthetic construct
GKNFQSRSHAEAAAKEAAAKAEAKYYKEVSSAATQIRYLPNLTAFQKAAFYAALLDDPSQSSELLSEAKKLNDSQAPK

SEQ ID NO: 110 - protein, 73 aa, synthetic construct
GKNFQSRSHGGGAAAAEAKYYKEVSSAATQIRYLPNLTAFQKAAFYAALLDDPSQSSELLSEAKKLNDSQAPK

SEQ ID NO: 111 - protein, 77 aa, synthetic construct
GSVSVSVKAEAAAKEAAAKAEAKYYKEVSSAATQIRYLPNLTAFQKAAFYAALLDDPSQSSELLSEAKKLNDSQAPK

SEQ ID NO: 112 - protein, 72 aa, synthetic construct
GSVSVSVKGGGAAAAEAKYYKEVSSAATQIRYLPNLTAFQKAAFYAALLDDPSQSSELLSEAKKLNDSQAPK

SEQ ID NO: 113 - DNA, 213 bp, synthetic construct
  source 1..213: mol_type = other DNA
ggcactaagaagaccctgcgcacgggcggaggggggtcggcagaagcgaaatacgcgaaagaacagttcaacgcctacgtcgtaatcttctacttaccaaacctgacagcatctcagaaagctgcgtttgttgatgcattaagtaatgatccatcccaaagctccgagttgctgagtgaggccaagaaacttaacgatagccaggctccgaag

SEQ ID NO: 114 - DNA, 234 bp, synthetic construct
  source 1..234: mol_type = other DNA
  repeat_region 1
ggaaccaagaagctgacattacgcaccgccgaggcggctgcgaaggaagcggctgccaaggccgaggcaaaatattacaaagaagttagctccgccgcaacgcaaattcgctatttacccaatttaacagcgttccagaaagccgcgttctacgctgccttgcttgatgatccgagccaaagttcggagctgttatcagaagcaaagaaactgaacgatagtcaagcgccaaaa

SEQ ID NO: 115 - DNA, 240 bp, synthetic construct
  source 1..240: mol_type = other DNA
gaagaggaagaggaggaggaggatggtgaagccgctgaggccgctgcaaaagaagctgcagctaaggccgaagctaagtattacaaggaggtaagttctgctgcgacacagatccgttatcttccgaatttaactgccttccaaaaggcggcgttctacgcggctcttttggatgatccatcacaatcgtccgaattgcttagcgaagcgaaaaaattgaatgactcccaagcgcccaag

SEQ ID NO: 116 - DNA, 234 bp, synthetic construct
  source 1..234: mol_type = other DNA
ggaaagaacttccaaagtcgtagtcatgctgaagccgcagctaaggaagctgcggcaaaggccgaagcaaaatactacaaagaagtgtcgagtgccgccacccaaatccgctatcttccgaacttgacagcatttcagaaggccgcattctacgccgccttgctggacgatccctctcagtcatcagagctgttaagcgaggcaaaaaaactgaatgattcacaggctccgaag

SEQ ID NO: 117 - DNA, 219 bp, synthetic construct
  source 1..219: mol_type = other DNA
gggaaaaactttcaatctcgtagccacggagggggtgcggcagcagcggaggcgaaatattacaaagaagttagttcagcggcaactcaaattcgctacttaccgaatttaacggcgtttcaaaaagccgcgttctatgccgccttactggacgaccctagtcaatcttctgagcttttgagtgaagccaaaaaattgaacgactcacaagcacctaag

SEQ ID NO: 118 - DNA, 231 bp, synthetic construct
  source 1..231: mol_type = other DNA
ggaagtgtaagtgtctcggtcaaagcagaagcagcggctaaagaggcagccgccaaagccgaggctaaatactataaggaagtatcaagcgcagcaactcaaatccgctacctgcctaacctgactgcgtttcaaaaggccgctttctatgcagcgttgttggatgaccctagccagtcgtccgagcttttaagtgaggcaaaaaaattaaatgattcccaagcgccgaag

SEQ ID NO: 119 - DNA, 216 bp, synthetic construct
  source 1..216: mol_type = other DNA
gggagtgtctctgttagtgtcaagggcggtggggcggcggctgcggaagccaagtattacaaggaagtctccagcgcagcaactcaaatccgctacttacccaatttaacagcatttcaaaaagccgccttttacgcggccctgcttgacgacccttctcaaagtagtgaacttttgagtgaggctaagaaactgaacgacagccaggcaccaaag

SEQ ID NO: 120 - protein, 5 aa, synthetic construct
GGGGS

SEQ ID NO: 121 - protein, 5 aa, synthetic construct
EAAAK

SEQ ID NO: 122 - protein, 12 aa, synthetic construct
AEAAAKEAAAKA

SEQ ID NO: 123 - protein, 8 aa, synthetic construct
XPXPXPXP

SEQ ID NO: 124 - protein, 10 aa, synthetic construct
GGGGSEAAAK

SEQ ID NO: 125 - protein, 7 aa, synthetic construct
GGGAAAA

SEQ ID NO: 126 - protein, 75 aa, synthetic construct
EEEEEEEDGEAGGGAAAAEAKYYKEVSSAATQIRYLPNLTAFQKAAFYAALLDDPSQSSELLSEAKKLNDSQAPK

SEQ ID NO: 127 - protein, 8 aa, synthetic construct
KNFQSRSH